(19) United States
(12) Patent Application Publication
(10) Pub. No.: US 2023/0217823 A1
(43) Pub. Date: Jul. 6, 2023

(54) ORGANIC ELECTROLUMINESCENT DEVICE

(71) Applicant: BEIJING SUMMER SPROUT TECHNOLOGY CO., LTD., BEIJING (CN)

(72) Inventors: Xuechao TIAN, Beijing (CN); Chunliang ZHAO, Beijing (CN); Bingjian GAO, Beijing (CN); Liuhuan CAI, Beijing (CN); Chi Yuen Raymond KWONG, Beijing (CN); Chuanjun XIA, Beijing (CN)

(21) Appl. No.: 18/090,235

(22) Filed: Dec. 28, 2022

(30) Foreign Application Priority Data
Dec. 31, 2021 (CN) 202111649394.X

(51) Int. Cl. H10K 85/60; H10K 85/30; C07D 495/04; C09K 11/06

(52) U.S. Cl. CPC H01L 51/0072; H01L 51/0071; H01L 51/008; C07D 495/04; C09K 11/06; H01L 51/5096

(57) ABSTRACT

Provided is an organic electroluminescent device. The organic electroluminescent device is a blue delayed fluorescent device including an anode, a cathode, an emissive layer disposed between the anode and the cathode and a hole blocking layer disposed between the emissive layer and the cathode, where the hole blocking layer comprises a hole blocking material having a structure of Formula 1, and the emissive layer comprises a first host material, a second host material and a blue delayed fluorescent material having a structure of Formula 3. The blue delayed fluorescent device exhibits excellent overall device performance, such as higher efficiency and longer lifetime. Further provided are a light-emitting device comprising a blue delayed fluorescent unit, a green phosphorescent unit and a red phosphorescent unit and a display assembly comprising the blue delayed fluorescent device and the light-emitting device.

### CROSS-REFERENCE TO RELATED APPLICATION(S)

This application claims priority to Chinese Patent Application No. 202111649394.X filed on Dec. 31, 2021, the disclosure of which is incorporated herein by reference in its entirety.

### TECHNICAL FIELD

The present disclosure relates to an organic electroluminescent device, for example, a delayed fluorescent device. More particularly, the present disclosure relates to a blue delayed fluorescent device comprising a hole blocking material having a structure of Formula 1 in a hole blocking layer and comprising a first host material, a second host material and a blue delayed fluorescent material in an emissive layer and a display assembly comprising the blue delayed fluorescent device, and a light-emitting device comprising a blue delayed fluorescent unit, a green phosphorescent unit and a red phosphorescent unit and a display assembly comprising the light-emitting device.

### BACKGROUND

Organic electroluminescent devices convert electrical energy into light by applying voltages across the devices. Generally, an organic electroluminescent device includes an anode, a cathode and an organic layer disposed between the anode and the cathode. The organic layer includes a hole injection layer (HIL), a hole transporting layer (HTL), an electron blocking layer (EBL), an emissive layer (EML, comprising a host material and a doped material), a hole blocking layer (HBL), an electron transporting layer (ETL), an electron injection layer (EIL) and the like. According to different functions of materials, the materials that constitute the organic layer may be divided into a hole injection material, a hole transporting material, an electron blocking material, a host material, a light-emitting material, a hole blocking material, an electron transporting material, an electron injection material and the like. When a bias voltage is applied to the device, holes are injected into the emissive layer from the anode, and electrons are injected into the emissive layer from the cathode. The holes and the electrons meet each other in the emissive layer to form excitons, and the excitons are recombined to emit light. In a thin film of an organic material, the mobility of electrons is generally much lower than that of holes, which may result in the accumulation of excess holes in the emissive layer, and the excess holes may result in the formation of positive ion compounds that do not emit light, thereby resulting in a reduction in device brightness and lifetime. To achieve a relatively good carrier balance, the hole blocking layer is introduced into the device.

The hole blocking layer is an important functional layer that affects performance of the organic electroluminescent device, and different organic electroluminescent devices have different requirements for the hole blocking material. A blue delayed fluorescent device has a very high requirement for an HBL material, and an excellent HBL material needs to have both a relatively high triplet energy level for hole blocking and a relatively good electron transporting capability, resulting in a limitation in a selection of the blue delayed fluorescent HBL material. A red phosphorescent device and a green phosphorescent device do not have such a high requirement for the triplet of the HBL and have a relatively large selection range of the HBL material. However, an HBL material which can be applied to blue delayed fluorescence may have a poor effect when applied to the red phosphorescent device and the green phosphorescent device, resulting in a reduction of overall performance of a light-emitting device comprising a red phosphorescent device, a green phosphorescent device and a blue delayed fluorescent device.

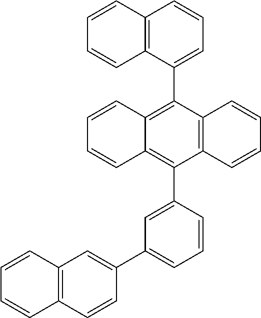 [SMILES: c1cc(-c2ccc3ccccc3c2)cc(-c2c3ccccc3c(-c3cccc4ccccc34)c3ccccc23)c1]

US20200343457A1 has disclosed a common fluorescent device with as a host and

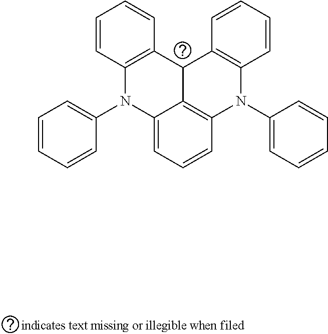 [SMILES: c1ccc(N2c3ccccc3C3c4ccccc4N(c4ccccc4)c4cccc2c43)cc1]

as a light-emitting material, and

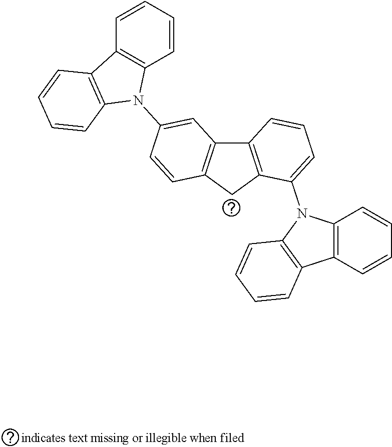 [SMILES: c1cc2c(c(-n3c4ccccc4c4ccccc43)c1)Cc1ccc(-n3c4ccccc4c4ccccc43)cc1-2]

is used as an HBL material in the device. However, a full text of this literature has not disclosed or taught that

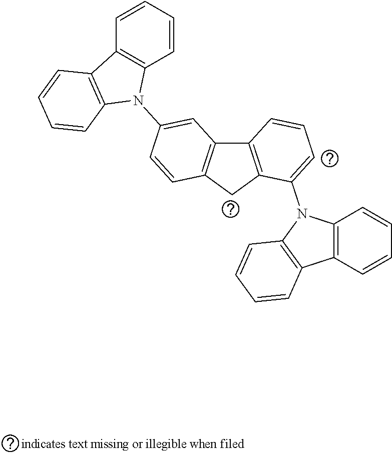 [SMILES: c1cc2c(c(-n3c4ccccc4c4ccccc43)c1)Cc1ccc(-n3c4ccccc4c4ccccc43)cc1-2]

can be used as an HBL material in a blue delayed fluorescent device, nor has the full text of this literature taught that the HBL material can be used in combination with two hosts having a high triplet energy level and can produce an effect, and nor has the full text of this literature disclosed or taught that the HBL material can be applied to a hole blocking layer in a light-emitting device comprising a red phosphorescent device, a green phosphorescent device and a blue delayed fluorescent device.

US20120049168A1 has disclosed a green phosphorescent device with

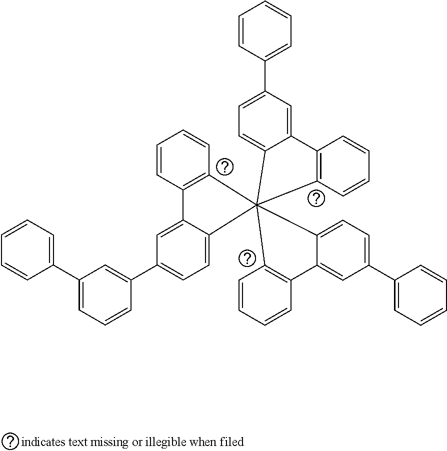

as a light-emitting material, and

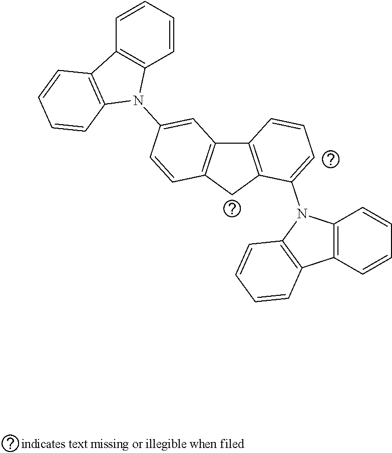 [SMILES: c1cc2c(c(-n3c4ccccc4c4ccccc43)c1)Cc1ccc(-n3c4ccccc4c4ccccc43)cc1-2]

is used as an HBL material in the device. This literature has not taught that the HBL material can be used in a hole blocking layer of a red phosphorescent device and a hole blocking layer of a blue delayed fluorescent device, nor has this literature disclosed or taught that the HBL material can be applied to a hole blocking layer in a light-emitting device comprising a red phosphorescent device, a green phosphorescent device and a blue delayed fluorescent device.

US20150295197A1 has disclosed a blue phosphorescent device with

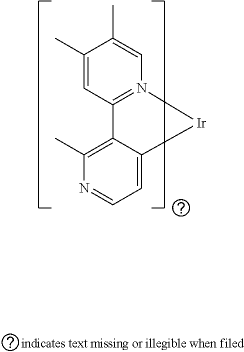

as a light-emitting material, and

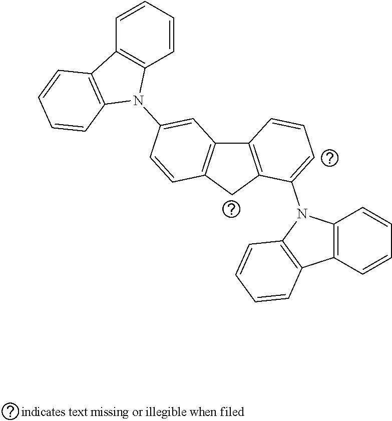 [SMILES: c1cc2c(c(-n3c4ccccc4c4ccccc43)c1)Cc1ccc(-n3c4ccccc4c4ccccc43)cc1-2]

is used as an HBL material in the device.

This literature is only limited to the use of the HBL material in the blue phosphorescent device and has not disclosed or taught that the HBL material can be used in a hole blocking layer of a red phosphorescent device, a hole blocking layer a green phosphorescent device and a hole blocking layer of a blue delayed fluorescent device, nor has this literature disclosed or taught that the HBL material can be applied to a hole blocking layer in a light-emitting device comprising a red phosphorescent device, a green phosphorescent device and a blue delayed fluorescent device.

US20160028023A1 has disclosed a red phosphorescent device with

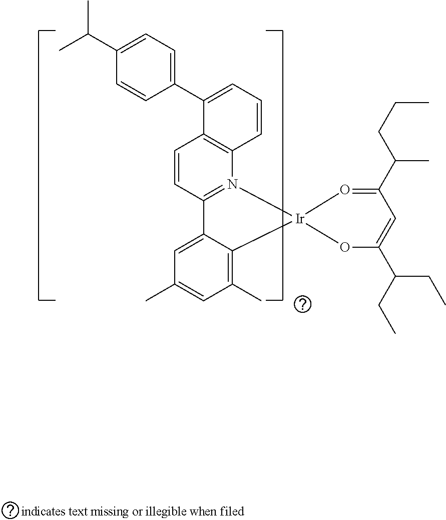

as a light-emitting material, and

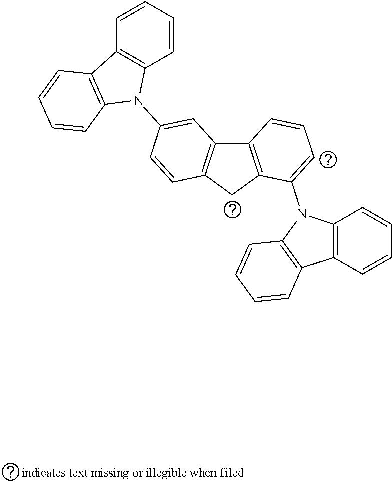 [SMILES: c1cc2c(c(-n3c4ccccc4c4ccccc43)c1)Cc1ccc(-n3c4ccccc4c4ccccc43)cc1-2]

is used as an HBL material in the device. This literature is only limited to the use of the HBL material in the red phosphorescent device and has not disclosed or taught that the HBL material can be used in a hole blocking layer of a green phosphorescent device and a hole blocking layer of a blue delayed fluorescent device, nor has this literature disclosed or taught that the HBL material can be applied to a hole blocking layer in a light-emitting device comprising a red phosphorescent device, a green phosphorescent device and a blue delayed fluorescent device.

The HBL material disclosed in the present disclosure not only has a good effect in the blue delayed fluorescent device of the present disclosure, but also has a relatively good effect in the red phosphorescent device and the green phosphorescent device. Therefore, the HBL material can also be applied to a hole blocking layer in the light-emitting device comprising a red phosphorescent device, a green phosphorescent device and a blue delayed fluorescent device, so as to improve the performance of the entire light-emitting device.

### SUMMARY

The present disclosure aims to provide a blue delayed fluorescent device to solve at least part of the above problems. The blue delayed fluorescent device comprises a hole blocking material having a structure of Formula 1 in a hole blocking layer, and comprises a first host material, a second host material and a blue delayed fluorescent material having a structure of Formula 3 in an emissive layer. The blue delayed fluorescent device can exhibit excellent overall device performance, such as higher efficiency and longer lifetime. The present disclosure further provides a light-emitting device to solve at least part of the above problems.

The light-emitting device comprises a blue delayed fluorescent device, a green phosphorescent device and a red phosphorescent device, where a hole blocking layer of the blue delayed fluorescent device, a hole blocking layer of the green phosphorescent device and a hole blocking layer of the red phosphorescent device each comprise a hole blocking material having a structure of Formula 1. The light-emitting device can reduce types of materials and improve performance of the entire light-emitting device, such as higher efficiency and longer lifetime.

According to an embodiment of the present disclosure, disclosed is a blue delayed fluorescent device, which comprises:

an anode,

a cathode,

an emissive layer disposed between the anode and cathode, and a hole blocking layer disposed between the emissive layer and the cathode, wherein the hole blocking layer comprises a hole blocking material;

wherein the hole blocking material is a compound having a structure represented by Formula 1:

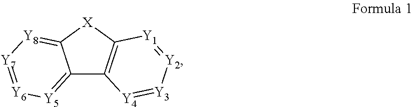

wherein in Formula 1,

X is selected from O, S, Se, SiRR or PR;

R is, at each occurrence identically or differently, selected from the group consisting of: substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted aryl having 6 to 30 carbon atoms, and substituted or unsubstituted heteroaryl having 3 to 30 carbon atoms; when multiple R are present at the same time, the multiple R are the same or different;

Y₁ to Y₈ are, at each occurrence identically or differently, selected from CR₁, CR₂ or N; at least one of Y₁ to Ys is selected from N, and at least one of Y₁ to Y₈ is selected from CR₁;

R₁ has a structure represented by Formula 2:

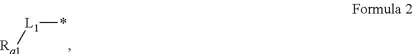

wherein Rₐ₁ is, at each occurrence identically or differently, selected from substituted or unsubstituted aryl having 6 to 30 carbon atoms, substituted or unsubstituted heteroaryl having 3 to 30 carbon atoms or a combination thereof;

L₁ is, at each occurrence identically or differently, selected from a single bond, substituted or unsubstituted arylene having 6 to 30 carbon atoms, substituted or unsubstituted heteroarylene having 3 to 30 carbon atoms or a combination thereof;

* represents a position where the substituent having the structure of Formula 2 is joined to Formula 1;

R₂ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;

adjacent substituents R, R₂ can be optionally joined to form a ring;

the emissive layer comprises a first host material, a second host material and a blue delayed fluorescent material, wherein the triplet energy level of the first host material and the triplet energy level of the second host material are each greater than that of the blue delayed fluorescent material;

the blue delayed fluorescent material has a structure represented by Formula 3:

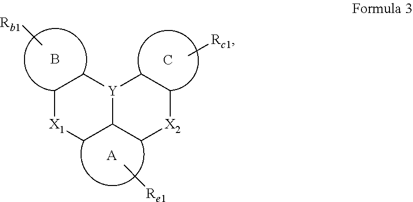

wherein in Formula 3,

the ring A, the ring B and the ring C are each independently selected from an aromatic ring having 6 to 30 carbon atoms, a heteroaromatic ring having 3 to 30 carbon atoms or a combination thereof;

Y is selected from B, P═O, P═S, Al, Ga, As, SiR′ or GeR′;

X₁ and X₂ are each independently selected from O, N—R_{d1}, S or Se, wherein N—R_{d1} has a structure represented by Formula 4:

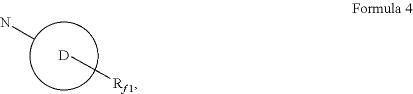

wherein the ring D is, at each occurrence identically or differently, selected from an aromatic ring having 6 to 30 carbon atoms, a heteroaromatic ring having 3 to 30 carbon atoms or a combination thereof;

R_{b1}, R_{c1}, Rₑ₁ and R_{f1} represent, at each occurrence identically or differently, mono-substitution, multiple substitutions or non-substitution;

R′, R_{b1}, R_{c1}, Rₑ₁ and R_{f1} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and

adjacent substituents R_{b1}, R_{c1}, Rₑ₁, R_{f1} can be optionally joined to form a ring.

According to another embodiment of the present disclosure, disclosed is a light-emitting device, which comprises an anode, a cathode and a light-emitting unit disposed between the anode and the cathode;

wherein the light-emitting unit at least comprises a blue delayed fluorescent unit, a green phosphorescent unit and a red phosphorescent unit;

wherein the blue delayed fluorescent unit, the green phosphorescent unit and the red phosphorescent unit each comprise an emissive layer and a hole blocking layer disposed between the emissive layer and the cathode;

wherein the hole blocking layers comprise a same hole blocking material or different hole blocking materials;

wherein the hole blocking material is a compound having a structure represented by Formula 1:

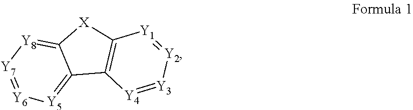

wherein in Formula 1,

X is selected from O, S, Se, SiRR or PR;

R is, at each occurrence identically or differently, selected from the group consisting of: substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted aryl having 6 to 30 carbon atoms, and substituted or unsubstituted heteroaryl having 3 to 30 carbon atoms; when multiple R are present at the same time, the multiple R are the same or different;

Y₁ to Y₈ are, at each occurrence identically or differently, selected from CR₁, CR₂ or N; at least one of Y₁ to Y₈ is selected from N, and at least one of Y₁ to Y₈ is selected from CR₁;

R₁ has a structure represented by Formula 2:

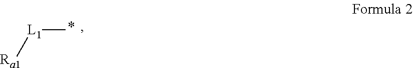

wherein Rₐ₁ is, at each occurrence identically or differently, selected from substituted or unsubstituted aryl having 6 to 30 carbon atoms, substituted or unsubstituted heteroaryl having 3 to 30 carbon atoms or a combination thereof;

L₁ is, at each occurrence identically or differently, selected from a single bond, substituted or unsubstituted arylene having 6 to 30 carbon atoms, substituted or unsubstituted heteroarylene having 3 to 30 carbon atoms or a combination thereof;

* represents a position where the substituent having the structure of Formula 2 is joined to Formula 1;

R₂ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;

adjacent substituents R, R₂ can be optionally joined to form a ring;

the emissive layer of the blue delayed fluorescent unit comprises a first host material, a second host material and a blue delayed fluorescent material, wherein the triplet energy level of the first host material and the triplet energy level of the second host material are each greater than that of the blue delayed fluorescent material;

the blue delayed fluorescent material has a structure represented by Formula 3:

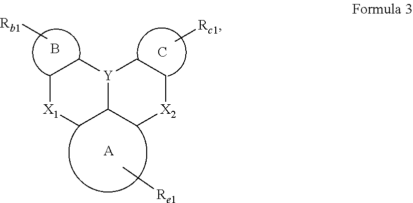

wherein in Formula 3,

the ring A, the ring B and the ring C are each independently selected from an aromatic ring having 6 to 30 carbon atoms, a heteroaromatic ring having 3 to 30 carbon atoms or a combination thereof;

Y is selected from B, P═O, P═S, Al, Ga, As, SiR′ or GeR′;

X₁ and X₂ are each independently selected from O, N—R_{d1}, S or Se, wherein N—R_{d1} has a structure represented by Formula 4:

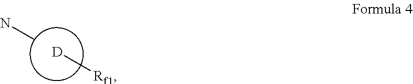

wherein the ring D is, at each occurrence identically or differently, selected from an aromatic ring having 6 to 30 carbon atoms, a heteroaromatic ring having 3 to 30 carbon atoms or a combination thereof;

R_{b1}, R_{c1}, Rₑ₁ and R_{f1} represent, at each occurrence identically or differently, mono-substitution, multiple substitutions or non-substitution;

R′, R_{b1}, R_{c1}, Rₑ₁ and R_{f1} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;

adjacent substituents R_{b1}, R_{c1}, Rₑ₁, R_{f1} can be optionally joined to form a ring;

the emissive layer of the green phosphorescent unit comprises a green phosphorescent material; and

the emissive layer of the red phosphorescent unit comprises a red phosphorescent material.

According to an embodiment of the present disclosure, disclosed is a display assembly, which comprises the device according to any one of the preceding embodiments.

According to an embodiment of the present disclosure, disclosed is a display assembly, which comprises the blue delayed fluorescent device according to the preceding embodiment.

According to an embodiment of the present disclosure, disclosed is a display assembly, which comprises the light-emitting device according to the preceding embodiment.

The present disclosure discloses a blue delayed fluorescent device, which comprises a hole blocking material having the structure of Formula 1 in a hole blocking layer, and comprises a first host material, a second host material and a blue delayed fluorescent material having the structure of Formula 3 in an emissive layer. The blue delayed fluorescent device can exhibit the excellent overall device performance, such as higher efficiency and longer lifetime. The present disclosure further discloses a light-emitting device, which comprises a cathode, an anode and a light-emitting unit disposed between the anode and the cathode, where the light-emitting unit at least comprises a blue delayed fluorescent unit, a green phosphorescent unit and a red phosphorescent unit, wherein the blue delayed fluorescent unit, the green phosphorescent unit and the red phosphorescent unit each comprise an emissive layer and a hole blocking layer disposed between the emissive layer and the cathode, wherein the hole blocking layers comprise the same hole blocking material or different hole blocking materials each having the structure of Formula 1. The hole blocking material(s) has(have) relatively good light-emitting performance in the above three (red, green and blue) light-emitting units. In particular, when the above three (red, green and blue) light-emitting units share a common hole blocking layer, the light-emitting device can reduce the types of the materials and improve the performance of the light-emitting device, such as higher efficiency and longer lifetime.

### BRIEF DESCRIPTION OF DRAWINGS

FIG. 1 is a schematic diagram of an organic electroluminescent device 100 disclosed herein.

FIG. 2 is a schematic diagram of another organic electroluminescent device 200 disclosed herein.

FIG. 3a is a schematic diagram of a structure 300 of an OLED device comprising a red light-emitting unit, a green light-emitting unit and a blue light-emitting unit.

FIG. 3b is a schematic diagram of a structure 400 of an OLED device comprising a red light-emitting unit, a green light-emitting unit and a blue light-emitting unit.

FIG. 3c is a schematic diagram of a structure 500 of an OLED device comprising a red light-emitting unit, a green light-emitting unit and a blue light-emitting unit.

### DETAILED DESCRIPTION

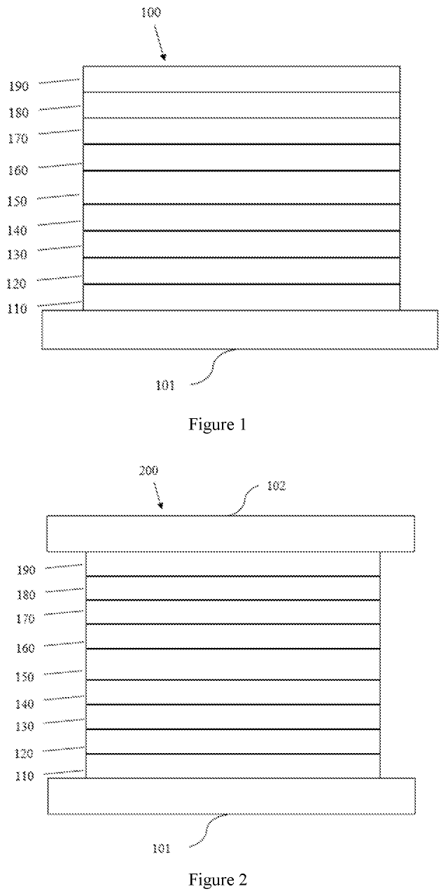

OLEDs can be fabricated on various types of substrates such as glass, plastic, and metal foil. FIG. 1 schematically shows an organic light-emitting device 100 without limitation. The figures are not necessarily drawn to scale. Some of the layers in the figures can also be omitted as needed. Device 100 may include a substrate 101, an anode 110, a hole injection layer 120, a hole transporting layer 130, an electron blocking layer 140, an emissive layer 150, a hole blocking layer 160, an electron transporting layer 170, an electron injection layer 180 and a cathode 190. Device 100 may be fabricated by depositing the layers described in order. The properties and functions of these various layers, as well as example materials, are described in more detail in U.S. Pat. No. 7,279,704 at cols. 6-10, the contents of which are incorporated by reference herein in its entirety.

More examples for each of these layers are available. For example, a flexible and transparent substrate-anode combination is disclosed in U.S. Pat. No. 5,844,363, which is incorporated by reference herein in its entirety. An example of a p-doped hole transporting layer is m-MTDATA doped with F4-TCNQ at a molar ratio of 50:1, as disclosed in U.S. Patent Application Publication No. 2003/0230980, which is incorporated by reference herein in its entirety. Examples of host materials are disclosed in U.S. Pat. No. 6,303,238 to Thompson et al., which is incorporated by reference herein in its entirety. An example of an n-doped electron transporting layer is BPhen doped with Li at a molar ratio of 1:1, as disclosed in U.S. Patent Application Publication No. 2003/0230980, which is incorporated by reference herein in its entirety. U.S. Pat. Nos. 5,703,436 and 5,707,745, which are incorporated by reference herein in their entireties, disclose examples of cathodes including composite cathodes having a thin layer of metal such as Mg:Ag with an overlying transparent, electrically-conductive, sputter-deposited ITO layer. The theory and use of blocking layers are described in more detail in U.S. Pat. No. 6,097,147 and U.S. Patent Application Publication No. 2003/0230980, which are incorporated by reference herein in their entireties. Examples of injection layers are provided in U.S. Patent Application Publication No. 2004/0174116, which is incorporated by reference herein in its entirety. A description of protective layers may be found in U.S. Patent Application Publication No. 2004/0174116, which is incorporated by reference herein in its entirety.

The layered structure described above is provided by way of non-limiting examples. Functional OLEDs may be achieved by combining the various layers described in different ways, or layers may be omitted entirely. It may also include other layers not specifically described. Within each layer, a single material or a mixture of multiple materials can be used to achieve optimum performance. Any functional layer may include several sublayers. For example, the emissive layer may have two layers of different emitting materials to achieve desired emission spectrum.

In one embodiment, an OLED may be described as having an “organic layer” disposed between a cathode and an anode. This organic layer may include a single layer or multiple layers.

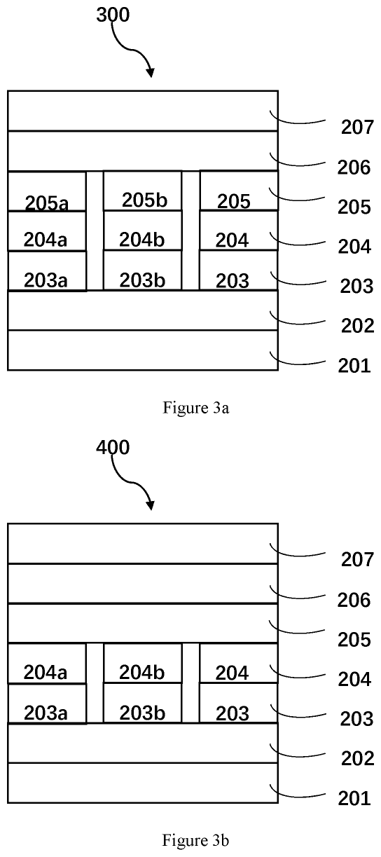

An OLED can be encapsulated by a barrier layer. FIG. 2 schematically shows an organic light emitting device 200 without limitation. FIG. 2 differs from FIG. 1 in that the organic light emitting device include a barrier layer 102, which is above the cathode 190, to protect it from harmful species from the environment such as moisture and oxygen. Any material that can provide the barrier function can be used as the barrier layer such as glass or organic-inorganic hybrid layers. The barrier layer should be placed directly or indirectly outside of the OLED device. Multilayer thin film encapsulation was described in U.S. Pat. No. 7,968,146, which is incorporated by reference herein in its entirety.

The “light-emitting unit” herein refers to a unit of an organic material layer that can emit light with a voltage or current applied, and the light-emitting unit may include one or more emissive layers. The light-emitting unit generally also includes one or more organic material layers to inject or transfer charge. For example, besides the emissive layer, the light-emitting unit may further include at least a hole injection layer, a hole transporting layer, an electron blocking layer, a hole blocking layer, an electron transporting layer and an electron injection layer. For example, in an example embodiment of the present disclosure, the light-emitting unit is composed of a hole injection layer, a hole transporting layer, an electron blocking layer, an emissive layer, a hole blocking layer and an electron transporting layer in sequence. An OLED device may be described as having a cathode, an anode and an “organic layer” disposed between the cathode and the anode, where the organic layer may include one or more layers, and a set of the “organic layer” is the “light-emitting unit”.

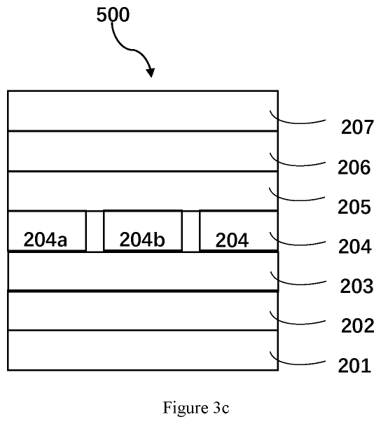

FIGS. 3a to 3c exemplarily illustrate, but are not limited to, an OLED device comprising a red light-emitting unit, a green light-emitting unit and a blue light-emitting unit. The structure 300 shown in FIG. 3a comprises three (red, green and blue) light-emitting units, 201 is a hole injection layer, 202 is a hole transporting layer, 203 is an electron blocking layer, 204 is an emissive layer, 205 is a hole blocking layer, 206 is an electron transporting layer, and 207 is an electron injection layer, where the emissive layer 204 is composed of 204a, 204b and 204c, and when assuming that 204a, 204b and 204c are a red emissive layer, a green emissive layer and a blue emissive layer, respectively, then corresponding 203a, 203b and 203c are electron blocking layers adapted to the red emissive layer, the green emissive layer and the blue emissive layer, respectively, and 205a, 205b and 205c are hole blocking layers adapted to the red emissive layer, the green emissive layer and the blue emissive layer, respectively. As shown in FIG. 3a, except that the electron blocking layer 203, the emissive layer 204 and the hole blocking layer 205 are different for the light-emitting units of different colors of red, green and blue, each of other layers is shared by the three light-emitting units. The other layers may also be different for the light-emitting units of the different colors of red, green and blue as needed, which are not described in detail herein. The structure 400 shown in FIG. 3b is another commonly used OLED device comprising a red light-emitting unit, a green light-emitting unit and a blue light-emitting unit. The structure 400 differs from the structure 300 shown in FIG. 3a is that the red light-emitting unit, the green light-emitting unit and the blue light-emitting unit in the structure 400 share a hole blocking layer. Since the same hole blocking layer is shared and the same hole blocking material is used, thereby reducing types of materials used and costs of the materials used. Further, the red light-emitting unit, the green light-emitting unit and the blue light-emitting unit may also share an electron blocking material, as needed, so that only the emissive layer is different, such as the structure 500 shown in FIG. 3c. Moreover, in some structures, the electron blocking layer 203 may be omitted.

Devices fabricated in accordance with embodiments of the present disclosure can be incorporated into a wide variety of consumer products that have one or more of the electronic component modules (or units) incorporated therein. Some examples of such consumer products include flat panel displays, monitors, medical monitors, televisions, billboards, lights for interior or exterior illumination and/or signaling, heads-up displays, fully or partially transparent displays, flexible displays, smart phones, tablets, phablets, wearable devices, smart watches, laptop computers, digital cameras, camcorders, viewfinders, micro-displays, 3-D displays, vehicles displays, and vehicle tail lights.

The materials and structures described herein may be used in other organic electronic devices listed above.

As used herein, “top” means furthest away from the substrate, while “bottom” means closest to the substrate. Where a first layer is described as “disposed over” a second layer, the first layer is disposed further away from the substrate. There may be other layers between the first and second layers, unless it is specified that the first layer is “in contact with” the second layer. For example, a cathode may be described as “disposed over” an anode, even though there are various organic layers in between.

As used herein, “solution processible” means capable of being dissolved, dispersed, or transported in and/or deposited from a liquid medium, either in solution or suspension form.

A ligand may be referred to as “photoactive” when it is believed that the ligand directly contributes to the photoactive properties of an emissive material. A ligand may be referred to as “ancillary” when it is believed that the ligand does not contribute to the photoactive properties of an emissive material, although an ancillary ligand may alter the properties of a photoactive ligand.

It is believed that the internal quantum efficiency (IQE) of fluorescent OLEDs can exceed the 25% spin statistics limit through delayed fluorescence. As used herein, there are two types of delayed fluorescence, i.e. P-type delayed fluorescence and E-type delayed fluorescence. P-type delayed fluorescence is generated from triplet-triplet annihilation (TTA).

On the other hand, E-type delayed fluorescence does not rely on the collision of two triplets, but rather on the transition between the triplet states and the singlet excited states. Compounds that are capable of generating E-type delayed fluorescence are required to have very small singlet-triplet gaps to convert between energy states. Thermal energy can activate the transition from the triplet state back to the singlet state. This type of delayed fluorescence is also known as thermally activated delayed fluorescence (TADF). A distinctive feature of TADF is that the delayed component increases as temperature rises. If the reverse intersystem crossing (RISC) rate is fast enough to minimize the non-radiative decay from the triplet state, the fraction of back populated singlet excited states can potentially reach 75%. The total singlet fraction can be 100%, far exceeding 25% of the spin statistics limit for electrically generated excitons.

E-type delayed fluorescence characteristics can be found in an exciplex system or in a single compound. Without being bound by theory, it is believed that E-type delayed fluorescence requires the luminescent material to have a small singlet-triplet energy gap (ΔE_{S-T}). Organic, non-metal containing, donor-acceptor luminescent materials may be able to achieve this. The emission in these materials is generally characterized as a donor-acceptor charge-transfer (CT) type emission. The spatial separation of the HOMO and LUMO in these donor-acceptor type compounds generally results in small Δ_{ES-T}. These states may involve CT states. Generally, donor-acceptor luminescent materials are constructed by connecting an electron donor moiety such as amino- or carbazole-derivatives and an electron acceptor moiety such as N-containing six-membered aromatic rings.

### Definition of Terms of Substituents

Halogen or halide—as used herein includes fluorine, chlorine, bromine, and iodine.

Alkyl—as used herein includes both straight and branched chain alkyl groups. Alkyl may be alkyl having 1 to 20 carbon atoms, preferably alkyl having 1 to 12 carbon atoms, and more preferably alkyl having 1 to 6 carbon atoms. Examples of alkyl groups include a methyl group, an ethyl group, a propyl group, an isopropyl group, a n-butyl group, an s-butyl group, an isobutyl group, a t-butyl group, an n-pentyl group, an n-hexyl group, an n-heptyl group, an n-octyl group, an n-nonyl group, an n-decyl group, an n-undecyl group, an n-dodecyl group, an n-tridecyl group, an n-tetradecyl group, an n-pentadecyl group, an n-hexadecyl group, an n-heptadecyl group, an n-octadecyl group, a neopentyl group, a 1-methylpentyl group, a 2-methylpentyl group, a 1-pentylhexyl group, a 1-butylpentyl group, a 1-heptyloctyl group, and a 3-methylpentyl group. Of the above, preferred are a methyl group, an ethyl group, a propyl group, an isopropyl group, a n-butyl group, an s-butyl group, an isobutyl group, a t-butyl group, an n-pentyl group, a neopentyl group, and an n-hexyl group. Additionally, the alkyl group may be optionally substituted.

Cycloalkyl—as used herein includes cyclic alkyl groups. The cycloalkyl groups may be those having 3 to 20 ring carbon atoms, preferably those having 4 to 10 carbon atoms. Examples of cycloalkyl include cyclobutyl, cyclopentyl, cyclohexyl, 4-methylcyclohexyl, 4,4-dimethylcylcohexyl, 1-adamantyl, 2-adamantyl, 1-norbornyl, 2-norbornyl, and the like. Of the above, preferred are cyclopentyl, cyclohexyl, 4-methylcyclohexyl, and 4,4-dimethylcylcohexyl. Additionally, the cycloalkyl group may be optionally substituted.

Heteroalkyl—as used herein, includes a group formed by replacing one or more carbons in an alkyl chain with a hetero-atom(s) selected from the group consisting of a nitrogen atom, an oxygen atom, a sulfur atom, a selenium atom, a phosphorus atom, a silicon atom, a germanium atom, and a boron atom. Heteroalkyl may be those having 1 to 20 carbon atoms, preferably those having 1 to 10 carbon atoms, and more preferably those having 1 to 6 carbon atoms. Examples of heteroalkyl include methoxymethyl, ethoxymethyl, ethoxyethyl, methylthiomethyl, ethylthiomethyl, ethylthioethyl, methoxymethoxymethyl, ethoxymethoxymethyl, ethoxyethoxyethyl, hydroxymethyl, hydroxyethyl, hydroxypropyl, mercaptomethyl, mercaptoethyl, mercaptopropyl, aminomethyl, aminoethyl, aminopropyl, dimethylaminomethyl, trimethylgermanylmethyl, trimethylgermanylethyl, trimethylgermanylisopropyl, dimethylethylgermanylmethyl, dimethylisopropylgermanylmethyl, tert-butylmethylgermanylmethyl, triethylgermanylmethyl, triethylgermanylethyl, triisopropylgermanylmethyl, triisopropylgermanylethyl, trimethylsilylmethyl, trimethylsilylethyl, and trimethylsilylisopropyl, triisopropylsilylmethyl, triisopropylsilylethyl. Additionally, the heteroalkyl group may be optionally substituted.

Alkenyl—as used herein includes straight chain, branched chain, and cyclic alkene groups. Alkenyl may be those having 2 to 20 carbon atoms, preferably those having 2 to 10 carbon atoms. Examples of alkenyl include vinyl, 1-propenyl group, 1-butenyl, 2-butenyl, 3-butenyl, 1,3-butandienyl, 1-methylvinyl, styryl, 2,2-diphenylvinyl, 1,2-diphenylvinyl, 1-methylallyl, 1,1-dimethylallyl, 2-methylallyl, 1-phenylallyl, 2-phenylallyl, 3-phenylallyl, 3,3-diphenylallyl, 1,2-dimethylallyl, 1-phenyl-1-butenyl, 3-phenyl-1-butenyl, cyclopentenyl, cyclopentadienyl, cyclohexenyl, cycloheptenyl, cycloheptatrienyl, cyclooctenyl, cyclooctatetraenyl, and norbornenyl. Additionally, the alkenyl group may be optionally substituted.

Alkynyl—as used herein includes straight chain alkynyl groups. Alkynyl may be those having 2 to 20 carbon atoms, preferably those having 2 to 10 carbon atoms. Examples of alkynyl groups include ethynyl, propynyl, propargyl, 1-butynyl, 2-butynyl, 3-butynyl, 1-pentynyl, 2-pentynyl, 3,3-dimethyl-1-butynyl, 3-ethyl-3-methyl-1-pentynyl, 3,3-diisopropyl-1-pentynyl, phenylethynyl, phenylpropynyl, etc. Of the above, preferred are ethynyl, propynyl, propargyl, 1-butynyl, 2-butynyl, 3-butynyl, 1-pentynyl, and phenylethynyl. Additionally, the alkynyl group may be optionally substituted.

Aryl or an aromatic group—as used herein includes non-condensed and condensed systems. Aryl may be those having 6 to 30 carbon atoms, preferably those having 6 to 20 carbon atoms, and more preferably those having 6 to 12 carbon atoms. Examples of aryl groups include phenyl, biphenyl, terphenyl, triphenylene, tetraphenylene, naphthalene, anthracene, phenalene, phenanthrene, fluorene, pyrene, chrysene, perylene, and azulene, preferably phenyl, biphenyl, terphenyl, triphenylene, fluorene, and naphthalene. Examples of non-condensed aryl groups include phenyl, biphenyl-2-yl, biphenyl-3-yl, biphenyl-4-yl, p-terphenyl-4-yl, p-terphenyl-3-yl, p-terphenyl-2-yl, m-terphenyl-4-yl, m-terphenyl-3-yl, m-terphenyl-2-yl, o-tolyl, m-tolyl, p-tolyl, p-(2-phenylpropyl)phenyl, 4′-methylbiphenylyl, 4″-t-butyl-p-terphenyl-4-yl, o-cumenyl, m-cumenyl, p-cumenyl, 2,3-xylyl, 3,4-xylyl, 2,5-xylyl, mesityl, and m-quarterphenyl. Additionally, the aryl group may be optionally substituted.

Heterocyclic groups or heterocycle—as used herein include non-aromatic cyclic groups. Non-aromatic heterocyclic groups include saturated heterocyclic groups having 3 to 20 ring atoms and unsaturated non-aromatic heterocyclic groups having 3 to 20 ring atoms, where at least one ring atom is selected from the group consisting of a nitrogen atom, an oxygen atom, a sulfur atom, a selenium atom, a silicon atom, a phosphorus atom, a germanium atom, and a boron atom. Preferred non-aromatic heterocyclic groups are those having 3 to 7 ring atoms, each of which includes at least one hetero-atom such as nitrogen, oxygen, silicon, or sulfur. Examples of non-aromatic heterocyclic groups include oxiranyl, oxetanyl, tetrahydrofuranyl, tetrahydropyranyl, dioxolanyl, dioxanyl, aziridinyl, dihydropyrrolyl, tetrahydropyrrolyl, piperidinyl, oxazolidinyl, morpholinyl, piperazinyl, oxepinyl, thiepinyl, azepinyl, and tetrahydrosilolyl. Additionally, the heterocyclic group may be optionally substituted.

Heteroaryl—as used herein, includes non-condensed and condensed hetero-aromatic groups having 1 to 5 hetero-atoms, where at least one hetero-atom is selected from the group consisting of a nitrogen atom, an oxygen atom, a sulfur atom, a selenium atom, a silicon atom, a phosphorus atom, a germanium atom, and a boron atom. A hetero-aromatic group is also referred to as heteroaryl. Heteroaryl may be those having 3 to 30 carbon atoms, preferably those having 3 to 20 carbon atoms, and more preferably those having 3 to 12 carbon atoms. Suitable heteroaryl groups include dibenzothiophene, dibenzofuran, dibenzoselenophene, furan, thiophene, benzofuran, benzothiophene, benzoselenophene, carbazole, indolocarbazole, pyridoindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, benzofuropyridine, furodipyridine, benzothienopyridine, thienodipyridine, benzoselenophenopyridine, and selenophenodipyridine, preferably dibenzothiophene, dibenzofuran, dibenzoselenophene, carbazole, indolocarbazole, imidazole, pyridine, triazine, benzimidazole, 1,2-azaborine, 1,3-azaborine, 1,4-azaborine, borazine, and aza-analogs thereof. Additionally, the heteroaryl group may be optionally substituted.

Alkoxy—as used herein, is represented by —O-alkyl, —O-cycloalkyl, —O-heteroalkyl, or —O-heterocyclic group. Examples and preferred examples of alkyl, cycloalkyl, heteroalkyl, and heterocyclic groups are the same as those described above. Alkoxy groups may be those having 1 to 20 carbon atoms, preferably those having 1 to 6 carbon atoms. Examples of alkoxy groups include methoxy, ethoxy, propoxy, butoxy, pentyloxy, hexyloxy, cyclopropyloxy, cyclobutyloxy, cyclopentyloxy, cyclohexyloxy, tetrahydrofuranyloxy, tetrahydropyranyloxy, methoxypropyloxy, ethoxyethyloxy, methoxymethyloxy, and ethoxymethyloxy. Additionally, the alkoxy group may be optionally substituted.

Aryloxy—as used herein, is represented by —O-aryl or —O-heteroaryl. Examples and preferred examples of aryl and heteroaryl are the same as those described above. Aryloxy groups may be those having 6 to 30 carbon atoms, preferably those having 6 to 20 carbon atoms. Examples of aryloxy groups include phenoxy and biphenyloxy. Additionally, the aryloxy group may be optionally substituted.

Arylalkyl—as used herein, contemplates alkyl substituted with an aryl group. Arylalkyl may be those having 7 to 30 carbon atoms, preferably those having 7 to 20 carbon atoms, and more preferably those having 7 to 13 carbon atoms. Examples of arylalkyl groups include benzyl, 1-phenylethyl, 2-phenylethyl, 1-phenylisopropyl, 2-phenylisopropyl, phenyl-t-butyl, alpha-naphthylmethyl, 1-alpha-naphthylethyl, 2-alpha-naphthylethyl, 1-alpha-naphthylisopropyl, 2-alpha-naphthylisopropyl, beta-naphthylmethyl, 1-beta-naphthylethyl, 2-beta-naphthylethyl, 1-beta-naphthylisopropyl, 2-beta-naphthylisopropyl, p-methylbenzyl, m-methylbenzyl, o-methylbenzyl, p-chlorobenzyl, m-chlorobenzyl, o-chlorobenzyl, p-bromobenzyl, m-bromobenzyl, o-bromobenzyl, p-iodobenzyl, m-iodobenzyl, o-iodobenzyl, p-hydroxybenzyl, m-hydroxybenzyl, o-hydroxybenzyl, p-aminobenzyl, m-aminobenzyl, o-aminobenzyl, p-nitrobenzyl, m-nitrobenzyl, o-nitrobenzyl, p-cyanobenzyl, m-cyanobenzyl, o-cyanobenzyl, 1-hydroxy-2-phenylisopropyl, and 1-chloro-2-phenylisopropyl. Of the above, preferred are benzyl, p-cyanobenzyl, m-cyanobenzyl, o-cyanobenzyl, 1-phenylethyl, 2-phenylethyl, 1-phenylisopropyl, and 2-phenylisopropyl. Additionally, the arylalkyl group may be optionally substituted.

Alkylsilyl—as used herein, contemplates a silyl group substituted with an alkyl group. Alkylsilyl groups may be those having 3 to 20 carbon atoms, preferably those having 3 to 10 carbon atoms. Examples of alkylsilyl groups include trimethylsilyl, triethylsilyl, methyldiethylsilyl, ethyldimethylsilyl, tripropylsilyl, tributylsilyl, triisopropylsilyl, methyldiisopropylsilyl, dimethylisopropylsilyl, tri-t-butylsilyl, triisobutylsilyl, dimethyl t-butylsilyl, and methyldi-t-butylsilyl. Additionally, the alkylsilyl group may be optionally substituted.

Arylsilyl—as used herein, contemplates a silyl group substituted with an aryl group. Arylsilyl groups may be those having 6 to 30 carbon atoms, preferably those having 8 to 20 carbon atoms. Examples of arylsilyl groups include triphenylsilyl, phenyldibiphenylylsilyl, diphenylbiphenylsilyl, phenyldiethylsilyl, diphenylethylsilyl, phenyldimethylsilyl, diphenylmethylsilyl, phenyldiisopropylsilyl, diphenylisopropylsilyl, diphenylbutylsilyl, diphenylisobutylsilyl, diphenyl t-butylsilyl. Additionally, the arylsilyl group may be optionally substituted.

Alkylgermanyl—as used herein contemplates a germanyl substituted with an alkyl group. The alkylgermanyl may be those having 3 to 20 carbon atoms, preferably those having 3 to 10 carbon atoms. Examples of alkylgermanyl include trimethylgermanyl, triethylgermanyl, methyldiethylgermanyl, ethyldimethylgermanyl, tripropylgermanyl, tributylgermanyl, triisopropylgermanyl, methyldiisopropylgermanyl, dimethylisopropylgermanyl, tri-t-butylgermanyl, triisobutylgermanyl, dimethyl-t-butylgermanyl, and methyldi-t-butylgermanyl. Additionally, the alkylgermanyl may be optionally substituted.

Arylgermanyl—as used herein contemplates a germanyl substituted with at least one aryl group or heteroaryl group. Arylgermanyl may be those having 6 to 30 carbon atoms, preferably those having 8 to 20 carbon atoms. Examples of arylgermanyl include triphenylgermanyl, phenyldibiphenylylgermanyl, diphenylbiphenylgermanyl, phenyldiethylgermanyl, diphenylethylgermanyl, phenyldimethylgermanyl, diphenylmethylgermanyl, phenyldiisopropylgermanyl, diphenylisopropylgermanyl, diphenylbutylgermanyl, diphenylisobutylgermanyl, and diphenyl-t-butylgermanyl. Additionally, the arylgermanyl may be optionally substituted.

The term “aza” in azadibenzofuran, azadibenzothiophene, etc. means that one or more of C—H groups in the respective aromatic fragment are replaced by a nitrogen atom. For example, azatriphenylene encompasses dibenzo[f,h]quinoxaline, dibenzo[f,h]quinoline and other analogs with two or more nitrogens in the ring system. One of ordinary skill in the art can readily envision other nitrogen analogs of the aza-derivatives described above, and all such analogs are intended to be encompassed by the terms as set forth herein.

In the present disclosure, unless otherwise defined, when any term of the group consisting of substituted alkyl, substituted cycloalkyl, substituted heteroalkyl, substituted heterocyclic group, substituted arylalkyl, substituted alkoxy, substituted aryloxy, substituted alkenyl, substituted alkynyl, substituted aryl, substituted heteroaryl, substituted alkylsilyl, substituted arylsilyl, substituted alkylgermanyl, substituted arylgermanyl, substituted amino, substituted acyl, substituted carbonyl, a substituted carboxylic acid group, a substituted ester group, substituted sulfinyl, substituted sulfonyl, and substituted phosphino is used, it means that any group of alkyl, cycloalkyl, heteroalkyl, heterocyclic group, arylalkyl, alkoxy, aryloxy, alkenyl, alkynyl, aryl, heteroaryl, alkylsilyl, arylsilyl, alkylgermanyl, arylgermanyl, amino, acyl, carbonyl, a carboxylic acid group, an ester group, sulfinyl, sulfonyl, and phosphino may be substituted with one or more groups selected from the group consisting of deuterium, halogen, unsubstituted alkyl having 1 to 20 carbon atoms, unsubstituted cycloalkyl having 3 to 20 ring carbon atoms, unsubstituted heteroalkyl having 1 to 20 carbon atoms, an unsubstituted heterocyclic group having 3 to 20 ring atoms, unsubstituted arylalkyl having 7 to 30 carbon atoms, unsubstituted alkoxy having 1 to 20 carbon atoms, unsubstituted aryloxy having 6 to 30 carbon atoms, unsubstituted alkenyl having 2 to 20 carbon atoms, unsubstituted alkynyl having 2 to 20 carbon atoms, unsubstituted aryl having 6 to 30 carbon atoms, unsubstituted heteroaryl having 3 to 30 carbon atoms, unsubstituted alkylsilyl having 3 to 20 carbon atoms, unsubstituted arylsilyl group having 6 to 20 carbon atoms, unsubstituted alkylgermanyl group having 3 to 20 carbon atoms, unsubstituted arylgermanyl group having 6 to 20 carbon atoms, unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof.

It is to be understood that when a molecular fragment is described as being a substituent or otherwise attached to another moiety, its name may be written as if it were a fragment (e.g. phenyl, phenylene, naphthyl, dibenzofuryl) or as if it were the whole molecule (e.g. benzene, naphthalene, dibenzofuran). As used herein, these different ways of designating a substituent or an attached fragment are considered to be equivalent.

In the compounds mentioned in the present disclosure, hydrogen atoms may be partially or fully replaced by deuterium. Other atoms such as carbon and nitrogen can also be replaced by their other stable isotopes. The replacement by other stable isotopes in the compounds may be preferred due to its enhancements of device efficiency and stability.

In the compounds mentioned in the present disclosure, multiple substitutions refer to a range that includes di-substitutions, up to the maximum available substitutions. When substitution in the compounds mentioned in the present disclosure represents multiple substitutions (including di-, tri-, and tetra-substitutions, etc.), that means the substituent may exist at a plurality of available substitution positions on its linking structure, the substituents present at a plurality of available substitution positions may have the same structure or different structures.

In the compounds mentioned in the present disclosure, adjacent substituents in the compounds cannot be joined to form a ring unless otherwise explicitly defined, for example, adjacent substituents can be optionally joined to form a ring. In the compounds mentioned in the present disclosure, the expression that adjacent substituents can be optionally joined to form a ring includes a case where adjacent substituents may be joined to form a ring and a case where adjacent substituents are not joined to form a ring. When adjacent substituents can be optionally joined to form a ring, the ring formed may be monocyclic or polycyclic (including spirocyclic, endocyclic, fused cyclic, and etc.), as well as alicyclic, heteroalicyclic, aromatic, or heteroaromatic. In such expression, adjacent substituents may refer to substituents bonded to the same atom, substituents bonded to carbon atoms which are directly bonded to each other, or substituents bonded to carbon atoms which are more distant from each other. Preferably, adjacent substituents refer to substituents bonded to the same carbon atom and substituents bonded to carbon atoms which are directly bonded to each other.

The expression that adjacent substituents can be optionally joined to form a ring is also intended to mean that two substituents bonded to the same carbon atom are joined to each other via a chemical bond to form a ring, which can be exemplified by the following formula:

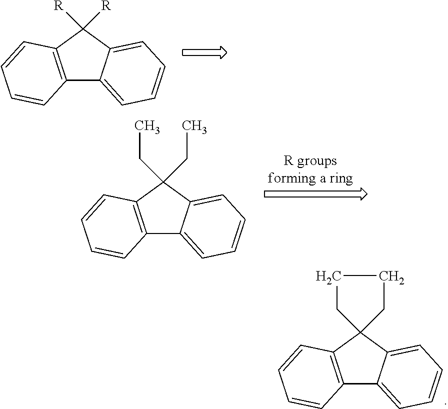

The expression that adjacent substituents can be optionally joined to form a ring is also intended to mean that two substituents bonded to carbon atoms which are directly bonded to each other are joined to each other via a chemical bond to form a ring, which can be exemplified by the following formula:

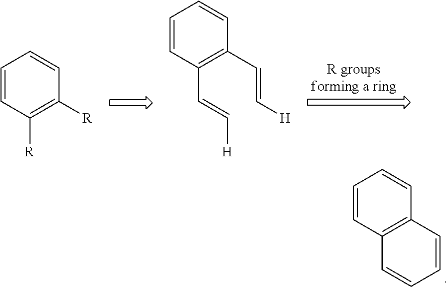

The expression that adjacent substituents can be optionally joined to form a ring is also intended to mean that two substituents bonded to further distant carbon atoms are joined to each other via a chemical bond to form a ring, which can be exemplified by the following formula:

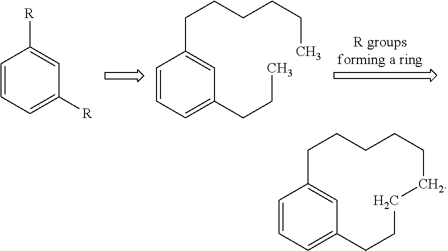

Furthermore, the expression that adjacent substituents can be optionally joined to form a ring is also intended to mean that, in the case where one of the two substituents bonded to carbon atoms which are directly bonded to each other represents hydrogen, the second substituent is bonded at a position at which the hydrogen atom is bonded, thereby forming a ring. This is exemplified by the following formula:

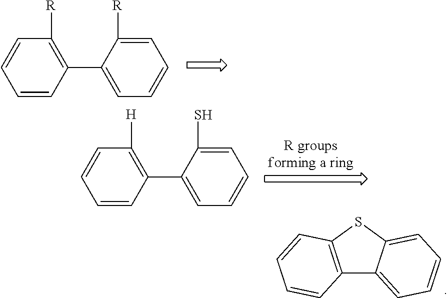

According to an embodiment of the present disclosure, disclosed is a blue delayed fluorescent device, which comprises:

an anode,

a cathode,

an emissive layer disposed between the anode and cathode, and a hole blocking layer disposed between the emissive layer and the cathode, wherein the hole blocking layer comprises a hole blocking material;

wherein the hole blocking material is a compound having a structure represented by Formula 1:

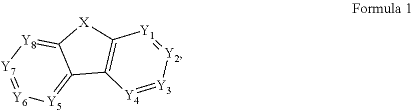

wherein in Formula 1,

X is selected from O, S, Se, SiRR or PR;

R is, at each occurrence identically or differently, selected from the group consisting of: substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted aryl having 6 to 30 carbon atoms, and substituted or unsubstituted heteroaryl having 3 to 30 carbon atoms; when multiple R are present at the same time, the multiple R are the same or different;

Y₁ to Y₈ are, at each occurrence identically or differently, selected from CR₁, CR₂ or N; at least one of Y₁ to Y₈ is selected from N, and at least one of Y₁ to Y₈ is selected from CR₁;

R₁ has a structure represented by Formula 2:

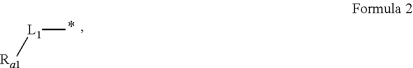

wherein Rₐ₁ is, at each occurrence identically or differently, selected from substituted or unsubstituted aryl having 6 to 30 carbon atoms, substituted or unsubstituted heteroaryl having 3 to 30 carbon atoms or a combination thereof;

L₁ is, at each occurrence identically or differently, selected from a single bond, substituted or unsubstituted arylene having 6 to 30 carbon atoms, substituted or unsubstituted heteroarylene having 3 to 30 carbon atoms or a combination thereof;

[symbol] represents a position where the substituent having the structure of Formula 2 is joined to Formula 1;

R₂ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;

adjacent substituents R, R₂ can be optionally joined to form a ring;

the emissive layer comprises a first host material, a second host material and a blue delayed fluorescent material, wherein the triplet energy level of the first host material and the triplet energy level of the second host material are each greater than that of the blue delayed fluorescent material;

the blue delayed fluorescent material has a structure represented by Formula 3:

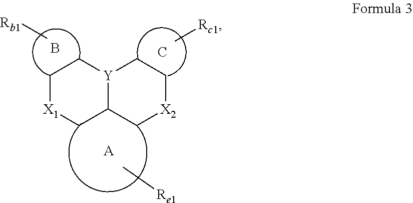

wherein in Formula 3,

the ring A, the ring B and the ring C are each independently selected from an aromatic ring having 6 to 30 carbon atoms, a heteroaromatic ring having 3 to 30 carbon atoms or a combination thereof;

Y is selected from B, P═O, P═S, Al, Ga, As, SiR′ or GeR′;

X₁ and X₂ are each independently selected from O, N—R_{d1}, S or Se, wherein N—R_{d1} has a structure represented by Formula 4:

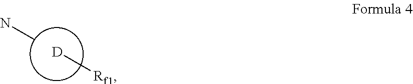

wherein the ring D is, at each occurrence identically or differently, selected from an aromatic ring having 6 to 30 carbon atoms, a heteroaromatic ring having 3 to 30 carbon atoms or a combination thereof;

R_{b1}, R_{c1}, Rₑ₁ and R_{f1} represent, at each occurrence identically or differently, mono-substitution, multiple substitutions or non-substitution;

R′, R_{b1}, R_{c1}, Rₑ₁ and R_{f1} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and

adjacent substituents R_{b1}, R_{c1}, Rₑ₁, R_{f1} can be optionally joined to form a ring.

In the present disclosure, the expression that adjacent substituents R, R₂ can be optionally joined to form a ring is intended to mean that any one or more of groups of adjacent substituents, such as two substituents R, and two substituents R₂, can be joined to form a ring.

Obviously, it is also possible that none of these groups of adjacent substituents are joined to form a ring.

In the present disclosure, the expression that adjacent substituents R_{b1}, R_{c1}, Rₑ₁, R_{f1} can be optionally joined to form a ring is intended to mean that any one or more of groups of adjacent substituents, such as two substituents R_{b1}, two substituents R_{c1}, two substituents Rₑ₁, two substituents R_{f1}, substituents R_{b1} and Rn, substituents Rₑ₁ and R_{f1}, and substituents Rₑ₁ and R_{f1}, can be joined to form a ring. Obviously, it is also possible that none of these groups of adjacent substituents are joined to form a ring.

According to an embodiment of the present disclosure, X is selected from O, S or Se.

According to an embodiment of the present disclosure, X is selected from O or S.

According to an embodiment of the present disclosure, X is selected from S.

According to an embodiment of the present disclosure, at least two of Y₁ to Y₈ are selected from CR₁, wherein R₁ has a structure represented by Formula 2:

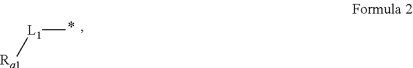

wherein Rₐ₁ is, at each occurrence identically or differently, selected from substituted or unsubstituted aryl having 6 to 30 carbon atoms, substituted or unsubstituted heteroaryl having 3 to 30 carbon atoms or a combination thereof;

L₁ is, at each occurrence identically or differently, selected from a single bond, substituted or unsubstituted arylene having 6 to 30 carbon atoms, substituted or unsubstituted heteroarylene having 3 to 30 carbon atoms or a combination thereof; and

[symbol] represents a position where the substituent having the structure of Formula 2 is joined to Formula 1.

According to an embodiment of the present disclosure, two and only two of Y₁ to Ys are selected from CR₁, wherein R₁ has a structure represented by Formula 2:

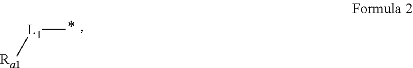

wherein Rₐ₁ is, at each occurrence identically or differently, selected from substituted or unsubstituted aryl having 6 to 30 carbon atoms, substituted or unsubstituted heteroaryl having 3 to 30 carbon atoms or a combination thereof;

L₁ is, at each occurrence identically or differently, selected from a single bond, substituted or unsubstituted arylene having 6 to 30 carbon atoms, substituted or unsubstituted heteroarylene having 3 to 30 carbon atoms or a combination thereof; and

[symbol] represents a position where the substituent having the structure of Formula 2 is joined to Formula 1.

According to an embodiment of the present disclosure, one and only one of Y₁ to Y₈ is selected from N.

According to an embodiment of the present disclosure, L₁ is, at each occurrence identically or differently, selected from substituted or unsubstituted arylene having 6 to 12 carbon atoms, substituted or unsubstituted heteroarylene having 3 to 12 carbon atoms or a combination thereof.

According to an embodiment of the present disclosure, L₁ is, at each occurrence identically or differently, selected from a single bond.

According to an embodiment of the present disclosure, Rₐ₁ is, at each occurrence identically or differently, selected from substituted or unsubstituted aryl having 6 to 24 carbon atoms, substituted or unsubstituted heteroaryl having 3 to 18 carbon atoms or a combination thereof.

According to an embodiment of the present disclosure, Rₐ₁ is, at each occurrence identically or differently, selected from the group consisting of: dibenzothienyl, dibenzofuryl, dibenzoselenophenyl, furyl, thienyl, benzofuryl, benzothienyl, benzoselenophenyl, carbazolyl, indolocarbazolyl, pyridoindolyl, pyrrolopyridinyl, pyrazolyl, imidazolyl, triazolyl, oxazolyl, thiazolyl, oxadiazolyl, oxatriazolyl, dioxazolyl, thiadiazolyl, pyridyl, pyridazinyl, pyrimidinyl, pyrazinyl, triazinyl, oxazinyl, oxathiazinyl, oxadiazinyl, indolyl, benzimidazolyl, indazolyl, indenoazinyl, benzoxazolyl, benzisoxazolyl, benzothiazolyl, quinolyl, isoquinolyl, cinnolinyl, quinazolinyl, quinoxalinyl, naphthyridinyl, phthalazinyl, pteridinyl, xanthenyl, acridinyl, phenazinyl, phenothiazinyl, benzofuropyridinyl, furodipyridinyl, benzothienopyridinyl, thienodipyridinyl, benzoselenophenopyridinyl and selenophenodipyridinyl.

According to an embodiment of the present disclosure, Rₐ₁ is, at each occurrence identically or differently, selected from the group consisting of: dibenzothienyl, dibenzofuryl, dibenzoselenophenyl, carbazolyl, indolocarbazolyl, imidazolyl, pyridyl, triazinyl, and benzimidazolyl.

According to an embodiment of the present disclosure, the hole blocking material is selected from the group consisting of Compound HB-1-1 to Compound HB-1-9, Compound HB-2-1 to Compound HB-2-9, Compound HB-3-1 to Compound HB-3-9, Compound HB-4-1 to Compound HB-4-9, Compound HB-5-1 to Compound HB-5-9, Compound HB-6-1 to Compound HB-6-9, Compound HB-7-1 to Compound HB-7-9, and Compound HB-8-1 to Compound HB-8-9, wherein the specific structures of Compound HB-1-1 to Compound HB-1-9, Compound HB-2-1 to Compound HB-2-9, Compound HB-3-1 to Compound HB-3-9, Compound HB-4-1 to Compound HB-4-9, Compound HB-5-1 to Compound HB-5-9, Compound HB-6-1 to Compound HB-6-9, Compound HB-7-1 to Compound HB-7-9, and Compound HB-8-1 to Compound HB-8-9 are referred to claim 7.

According to an embodiment of the present disclosure, at least one of X₁ and X₂ is selected from N—R_{d1}, wherein N—R_{d1} has a structure represented by Formula 4:

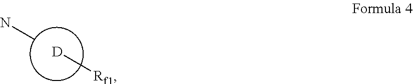

wherein the ring D is, at each occurrence identically or differently, selected from an aromatic ring having 6 to 30 carbon atoms, a heteroaromatic ring having 3 to 30 carbon atoms or a combination thereof;

R_{f1} represents, at each occurrence identically or differently, mono-substitution, multiple substitutions or non-substitution; and

R_{f1} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof.

According to an embodiment of the present disclosure, wherein the blue delayed fluorescent material has a structure represented by Formula 5:

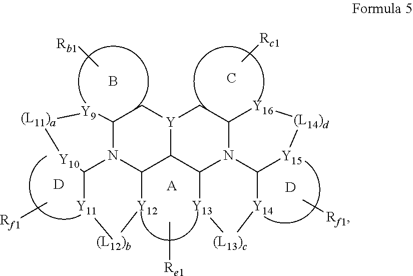

wherein in Formula 5,

the ring A, the ring B, the ring C and the ring D are, at each occurrence identically or differently, selected from an aromatic ring having 6 to 30 carbon atoms, a heteroaromatic ring having 3 to 30 carbon atoms or a combination thereof;

Y is selected from B, P═O, P═S, Al, Ga, As, SiR′ or GeR′;

Y₉ to Y₁₆ are each independently selected from C, CR″ or N;

a, b, c and d are each independently selected from 0 or 1;

L₁₁, L₁₂, L₁₃ and L₁₄ are, at each occurrence identically or differently, selected from a single bond, O, S or NR′″;

R_{b1}, R_{c1}, Rₑ₁ and R_{f1} represent, at each occurrence identically or differently, mono-substitution, multiple substitutions or non-substitution;

R′, R″, R′″, R_{b1}, R_{c1}, Rₑ₁ and R_{f1} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and

adjacent substituents R_{b1}, R_{c1}, Rₑ₁, R_{f1} can be optionally joined to form a ring.

According to an embodiment of the present disclosure, wherein the ring A, the ring B, the ring C and the ring D are, at each occurrence identically or differently, selected from an aromatic ring having 6 to 18 carbon atoms, a heteroaromatic ring having 3 to 18 carbon atoms or a combination thereof.

According to an embodiment of the present disclosure, the ring A, the ring B, the ring C and the ring D are, at each occurrence identically or differently, selected from a benzene ring, a pyridine ring, a carbazole ring, an N-phenylcarbazole ring, a dibenzofuran ring or a dibenzothiophene ring.

According to an embodiment of the present disclosure, the ring A, the ring B, the ring C and the ring D are, at each occurrence identically or differently, selected from a benzene ring.

According to an embodiment of the present disclosure, Y is selected from B, P═O or P═S.

According to an embodiment of the present disclosure, Y is B.

According to an embodiment of the present disclosure, in Formula 5, a+b+c+d is greater than or equal to 1.

According to an embodiment of the present disclosure, in Formula 5, a+b+c+d is greater than or equal to 2.

According to an embodiment of the present disclosure, in Formula 5, a is 1, b is 0, c is 1, and d is 0.

According to an embodiment of the present disclosure, in Formula 5, a is 1, b is 0, c is 0, and d is 1.

According to an embodiment of the present disclosure, in Formula 5, a is 1, b is 0, c is 1, d is 0, and L₁₁ and L₁₃ are selected from a single bond.

According to an embodiment of the present disclosure, in Formula 5, a is 1, b is 0, c is 0, d is 1, and L₁₁ and L₁₄ are selected from a single bond.

According to an embodiment of the present disclosure, wherein R_{b1}, R_{c1}, Rₑ₁ and R_{f1} are, at each occurrence identically or differently, selected from the group consisting of: hydrogen, deuterium, fluorine, methyl, deuterated methyl, ethyl, n-propyl, isopropyl, cyclopropyl, n-butyl, isobutyl, t-butyl, cyclopentyl, cyclohexyl, phenyl, trimethylsilyl, carbazolyl, indolyl, benzofuryl, dibenzofuryl, benzosilolyl, dibenzosilolyl, benzothienyl, dibenzothienyl, dibenzoselenophene and combinations thereof.

According to an embodiment of the present disclosure, wherein the blue delayed fluorescent material is selected from the group consisting of Compound BD1 to Compound BD37, wherein the specific structures of Compound BD1 to Compound BD37 are referred to claim 13.

According to an embodiment of the present disclosure, wherein the triplet energy level of the first host material and the triplet energy level of the second host material are each greater than 2.69 eV.

According to an embodiment of the present disclosure, wherein the first host material has a structure represented by any one of Formula 6 to Formula 8:

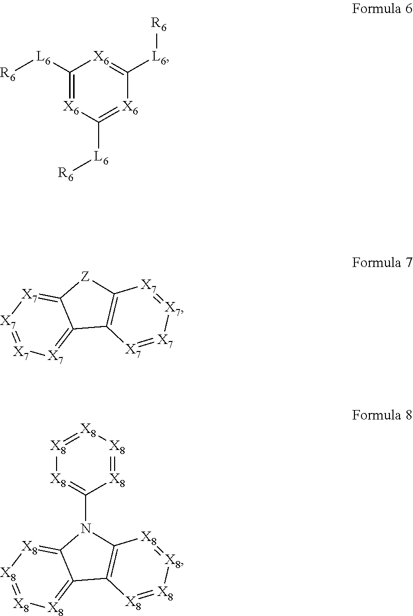

wherein,

X₆, X₇ and X₈ shown in Formula 6 to Formula 8 are, at each occurrence identically or differently, selected from CR₇ or N; at least one of X₆ shown in Formula 6 is selected from N, at least one of X₇ shown in Formula 7 is selected from N, and at least one of X₈ shown in Formula 8 is selected from N;

Z is, at each occurrence identically or differently, selected from O or S;

L₆ is, at each occurrence identically or differently, selected from the group consisting of: a single bond, substituted or unsubstituted arylene having 6 to 30 carbon atoms, substituted or unsubstituted heteroarylene having 3 to 30 carbon atoms and combinations thereof;

R₆ and R₇ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and

adjacent substituents R₇ can be optionally joined to form a ring.

In this embodiment, the expression that adjacent substituents R₇ can be optionally joined to form a ring is intended to mean that: two substituents R₇ in Formula 6 can be joined to form a ring (obviously, it is also possible that two substituents R₇ in Formula 6 are not joined to form a ring); two substituents R₇ in Formula 7 can be joined to form a ring (obviously, it is also possible that two substituents R₇ in Formula 7 are not joined to form a ring); and two substituents R₇ in Formula 8 can be joined to form a ring (obviously, it is also possible that two substituents R₇ in Formula 8 are not joined to form a ring).

According to an embodiment of the present disclosure, wherein the first host material is selected from the group consisting of Compound HB-3-1, Compound HB-5-1, Compound HB-6-1 and Compound N-1 to Compound N-7, wherein the specific structures of Compound HB-3-1, Compound HB-5-1, Compound HB-6-1 and Compound N-1 to Compound N-7 are referred to claim 16.

According to an embodiment of the present disclosure, the second host material has a structure represented by Formula 9:

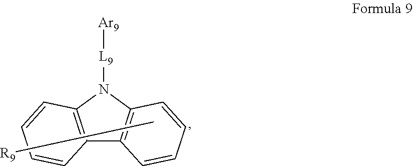

wherein in Formula 9,

L₉ is selected from a single bond, substituted or unsubstituted arylene having 6 to 30 carbon atoms, substituted or unsubstituted heteroarylene having 3 to 30 carbon atoms or a combination thereof;

Ar₉ is selected from substituted or unsubstituted aryl having 6 to 30 carbon atoms, substituted or unsubstituted heteroaryl having 3 to 30 carbon atoms, substituted or unsubstituted amino having 0 to 30 carbon atoms or a combination thereof;

R₉ represents, at each occurrence identically or differently, mono-substitution, multiple substitutions or non-substitution;

R₉ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and

adjacent substituents R₉ can be optionally joined to form a ring.

In this embodiment, the expression that adjacent substituents R₉ can be optionally joined to form a ring is intended to mean that two adjacent substituents R₉ can be joined to form a ring. Obviously, it is also possible that two adjacent substituents R₉ are not joined to form a ring.

According to an embodiment of the present disclosure, wherein the second host material is selected from the group consisting of Compound P-1 to Compound P-9, wherein the specific structures of Compound P-1 to Compound P-9 are referred to claim 18.

According to an embodiment of the present disclosure, further disclosed is a light-emitting device, which comprises an anode, a cathode and a light-emitting unit disposed between the anode and the cathode;

wherein the light-emitting unit at least comprises a blue delayed fluorescent unit, a green phosphorescent unit and a red phosphorescent unit;

wherein the blue delayed fluorescent unit, the green phosphorescent unit and the red phosphorescent unit each comprise an emissive layer and a hole blocking layer disposed between the emissive layer and the cathode;

wherein the hole blocking layers comprise a same hole blocking material or different hole blocking materials;

wherein the hole blocking material is a compound having a structure represented by Formula 1:

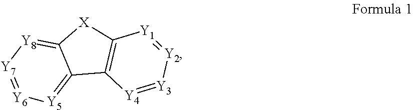

wherein in Formula 1,

X is selected from O, S, Se, SiRR or PR;

R is, at each occurrence identically or differently, selected from the group consisting of: substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted aryl having 6 to 30 carbon atoms, and substituted or unsubstituted heteroaryl having 3 to 30 carbon atoms; when multiple R are present at the same time, the multiple R are the same or different;

Y₁ to Y₈ are, at each occurrence identically or differently, selected from CR₁, CR₂ or N; at least one of Y₁ to Y₈ is selected from N, and at least one of Y₁ to Y₈ is selected from CR₁;

R₁ has a structure represented by Formula 2:

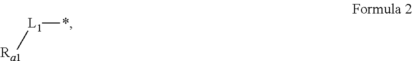

wherein Rₐ₁ is, at each occurrence identically or differently, selected from substituted or unsubstituted aryl having 6 to 30 carbon atoms, substituted or unsubstituted heteroaryl having 3 to 30 carbon atoms or a combination thereof;

L₁ is, at each occurrence identically or differently, selected from a single bond, substituted or unsubstituted arylene having 6 to 30 carbon atoms, substituted or unsubstituted heteroarylene having 3 to 30 carbon atoms or a combination thereof;

[symbol] represents a position where the substituent having the structure of Formula 2 is joined to Formula 1;

R₂ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;

adjacent substituents R, R₂ can be optionally joined to form a ring;

the emissive layer of the blue delayed fluorescent unit comprises a first host material, a second host material and a blue delayed fluorescent material, wherein the triplet energy level of the first host material and the triplet energy level of the second host material are each greater than that of the blue delayed fluorescent material;

the blue delayed fluorescent material has a structure represented by Formula 3:

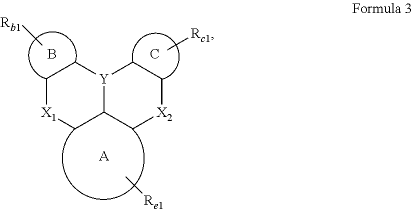

wherein in Formula 3,

the ring A, the ring B and the ring C are each independently selected from an aromatic ring having 6 to 30 carbon atoms, a heteroaromatic ring having 3 to 30 carbon atoms or a combination thereof;

Y is selected from B, P═O, P═S, Al, Ga, As, SiR′ or GeR′;

X₁ and X₂ are each independently selected from O, N—R_{d1}, S or Se, wherein N—R_{d1} has a structure represented by Formula 4:

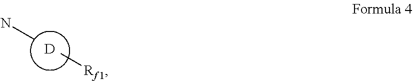

wherein the ring D is, at each occurrence identically or differently, selected from an aromatic ring having 6 to 30 carbon atoms, a heteroaromatic ring having 3 to 30 carbon atoms or a combination thereof;

R_{b1}, R_{c1}, Rₑ₁ and R_{f1} represent, at each occurrence identically or differently, mono-substitution, multiple substitutions or non-substitution;

R′, R_{b1}, R_{c1}, Rₑ₁ and R_{f1} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;

adjacent substituents R_{b1}, R_{c1}, Rₑ₁, R_{f1} can be optionally joined to form a ring;

the emissive layer of the green phosphorescent unit comprises a green phosphorescent material; and

the emissive layer of the red phosphorescent unit comprises a red phosphorescent material.

According to an embodiment of the present disclosure, wherein the blue delayed fluorescent unit, the green phosphorescent unit and the red phosphorescent unit share a hole blocking layer, wherein the hole blocking layer comprises a same hole blocking material.

According to an embodiment of the present disclosure, wherein the green phosphorescent material has a structure represented by Formula 10:

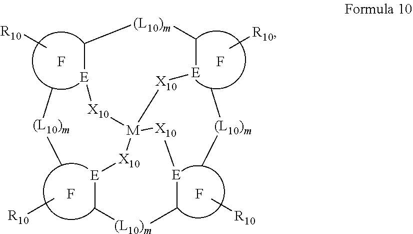

wherein,

the metal M is, at each occurrence identically or differently, selected from the group consisting of Cu, Ag, Au, Ru, Rh, Pd, Os, Ir and Pt;

the ring F is, at each occurrence identically or differently, selected from a substituted or unsubstituted aromatic ring having 5 to 30 ring atoms, a substituted or unsubstituted heteroaromatic ring having 5 to 30 ring atoms or a combination thereof;

L₁₀ is, at each occurrence identically or differently, selected from the group consisting of: a single bond, BR₁₁, CR₁₁R₁₁, NR₁₁, O, SiR₁₁R₁₁, PR₁₁, S, GeR₁₁R₁₁, Se, substituted or unsubstituted vinylene, ethynylene, substituted or unsubstituted arylene having 5 to 30 carbon atoms, substituted or unsubstituted heteroarylene having 5 to 30 carbon atoms and combinations thereof; when two R₁₁ are present at the same time, the two R₁₁ are the same or different;

m is, at each occurrence identically or differently, selected from 0 or 1; when m=0, the rings F are not joined to each other;

E is, at each occurrence identically or differently, selected from C or N;

X₁₀ is, at each occurrence identically or differently, selected from a single bond, O or S;

R₁₀ represents, at each occurrence identically or differently, mono-substitution, multiple substitutions or non-substitution;

R₁₀ and R₁₁ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and

adjacent substituents R₁₀, R₁₁ can be optionally joined to form a ring.

In this embodiment, the expression that adjacent substituents R₁₀, R₁₁ can be optionally joined to form a ring is intended to mean that any one or more of groups of adjacent substituents, such as two substituents R₁₀, two substituents Ru, and substituents R₁₀ and R₁₁, can be joined to form a ring. Obviously, it is also possible that none of these groups of adjacent substituents are joined to form a ring.

According to an embodiment of the present disclosure, wherein the green phosphorescent material has a general formula of M(Lₐ₁)_{f}(L_{b1})_{g}(Lₑ₁)ₕ;

the metal M is, at each occurrence identically or differently, selected from the group consisting of Cu, Ag, Au, Ru, Rh, Pd, Os, Ir and Pt;

Lₐ₁, L_{b1} and L_{c1} are a first ligand, a second ligand and a third ligand coordinated to the metal M, respectively, wherein Lₐ₁, L_{b1} and L_{c1} can be optionally joined to form a multidentate ligand;

f is selected from 0, 1, 2 or 3, g is selected from 0, 1, 2 or 3, and h is selected from 0, 1 or 2;

when f is 2 or 3, multiple Lₐ are the same or different; when g is 2 or 3, multiple L_{b} are the same or different; when h is 2, two L_{c1} are the same or different;

Lₐ₁ has a structure represented by Formula 11:

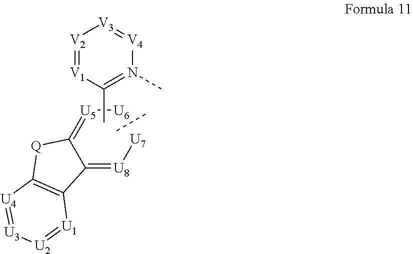

wherein,

Q is, at each occurrence identically or differently, selected from the group consisting of O, S, Se, NR_{q}, CR_{q}R_{q} and SiR_{q}R_{q}; when two R_{q} are present at the same time, the two R_{q} are the same or different;

U₁ to U₈ are, at each occurrence identically or differently, selected from C, CRᵤ or N;

U₅, U₆, U₇ or U₈ is joined to the metal M by a metal-carbon bond or a metal-nitrogen bond;

V₁ to V₄ are, at each occurrence identically or differently, selected from CRᵥ or N;

R_{q}, Rᵤ and Rᵥ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, a cyano group, an isocyano group, a sulfanyl group, a hydroxyl group, a sulfinyl group, a sulfonyl group, a phosphino group and combinations thereof;

adjacent substituents R_{q}, Rᵤ and Rᵥ can be optionally joined to form a ring;

L_{b1} and L_{c1} are, at each occurrence identically or differently, selected from a structure represented by any one of the group consisting of the following:

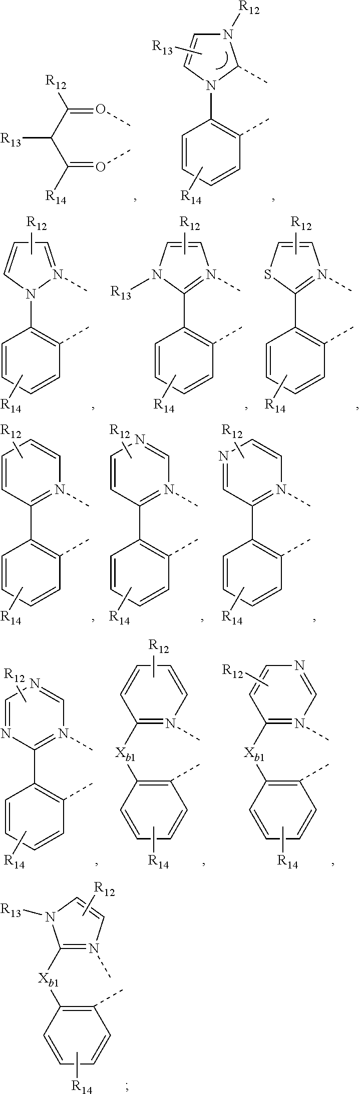

wherein,

R₁₂, R₁₃ and R₁₄ represent, at each occurrence identically or differently, mono-substitution, multiple substitutions or non-substitution;

X_{b1} is, at each occurrence identically or differently, selected from the group consisting of: O, S, Se, NR_{N1} and CR_{C1}R_{C2};

R₁₂, R₁₃, R₁₄, R_{N1}, R_{C1} and R_{C2} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and

adjacent substituents R₁₂, R₁₃, R₁₄, R_{N1}, R_{C1} and R_{C2} can be optionally joined to form a ring.

In this embodiment, the expression that adjacent substituents R_{q}, Rᵤ and Rᵥ can be optionally joined to form a ring is intended to mean that any one or more of groups of adjacent substituents, such as two substituents R_{q}, two substituents Rᵤ, two substituents Rᵥ, substituents R_{q} and Rᵤ, and substituents Rᵤ and Rᵥ can be joined to form a ring. Obviously, it is also possible that none of these groups of adjacent substituents are joined to form a ring.

In this embodiment, the expression that adjacent substituents R₁₂, R₁₃, R₁₄, R_{N1}, R_{C1} and R_{C2} can be optionally joined to form a ring is intended to mean that any one or more of groups of adjacent substituents, such as two substituents R₁₂, two substituents R₁₃, two substituents R₁₄, substituents R₁₂ and R₁₃, substituents R₁₃ and R₁₄, substituents R₁₂ and R_{N1}, substituents R₁₃ and R_{N1}, substituents R₁₄ and R_{N1}, substituents R₁₂ and R_{C1}, substituents R₁₃ and R_{C1}, substituents R₁₄ and R_{C1}, substituents R₁₂ and R_{C2}, substituents R₁₃ and R_{C2}, and substituents R₁₄ and R_{C2}, can be joined to form a ring. Obviously, it is also possible that none of these groups of adjacent substituents are joined to form a ring.

According to an embodiment of the present disclosure, wherein the green phosphorescent material is selected from the group consisting of the following:

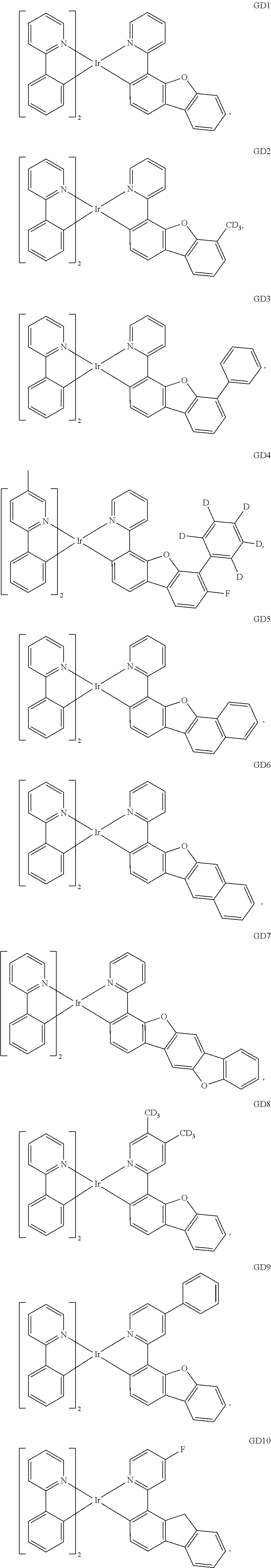

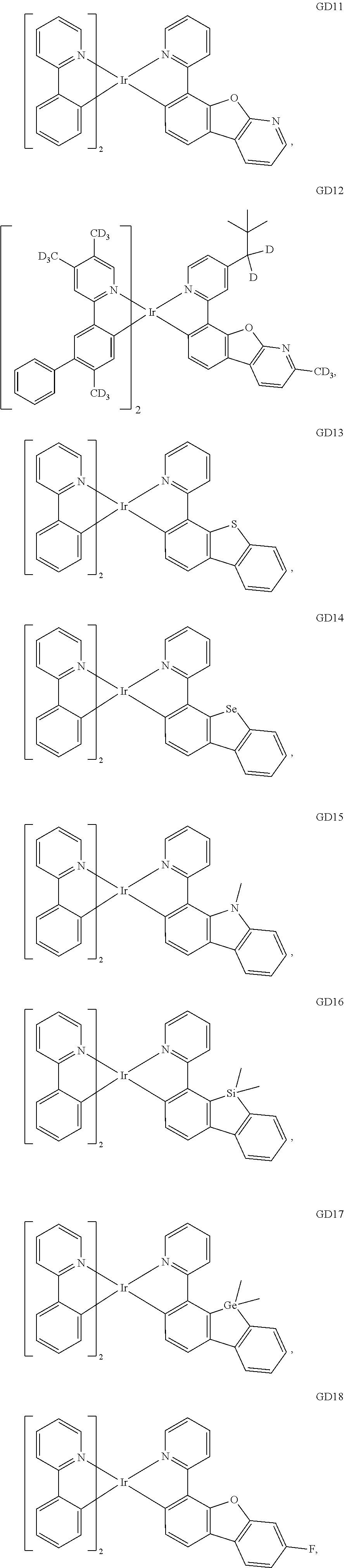

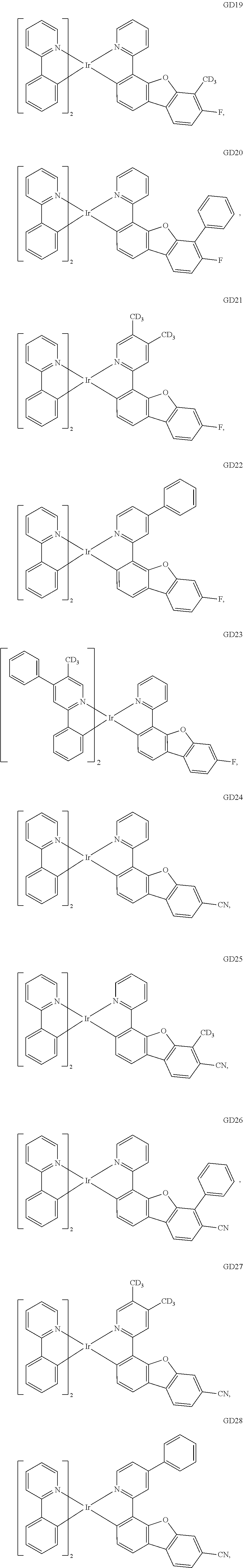

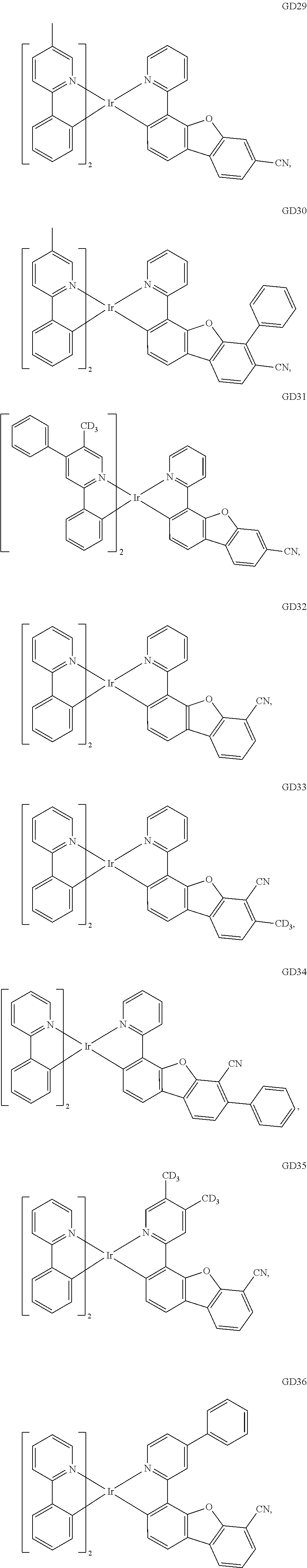

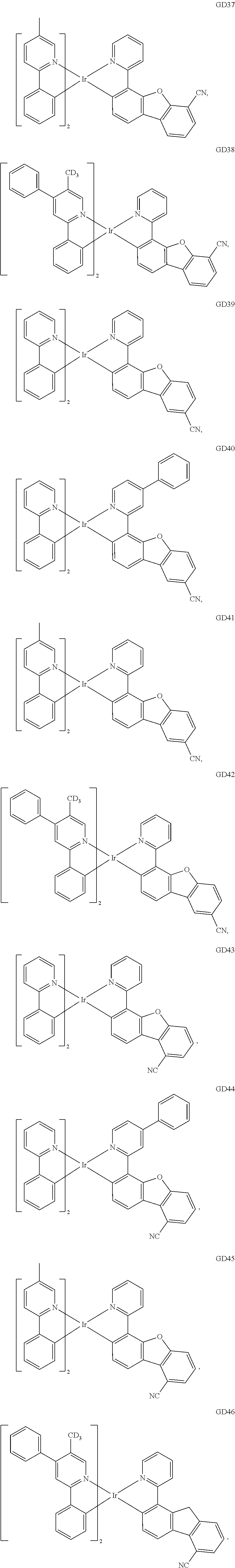

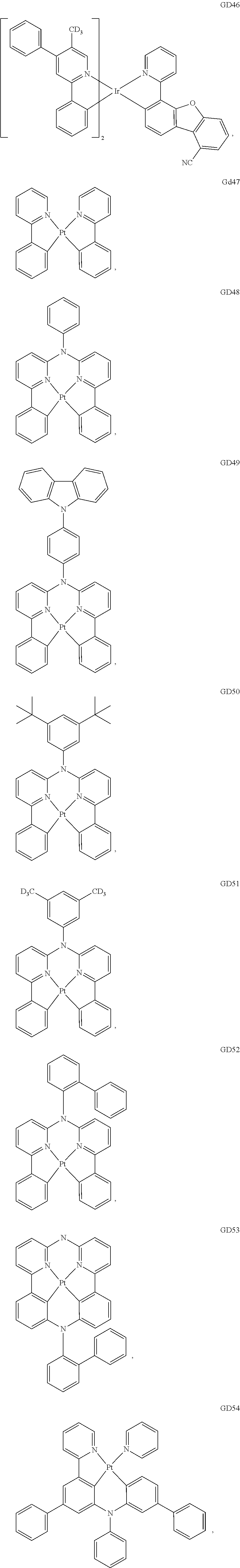

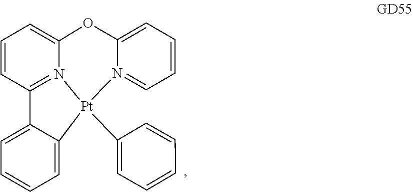

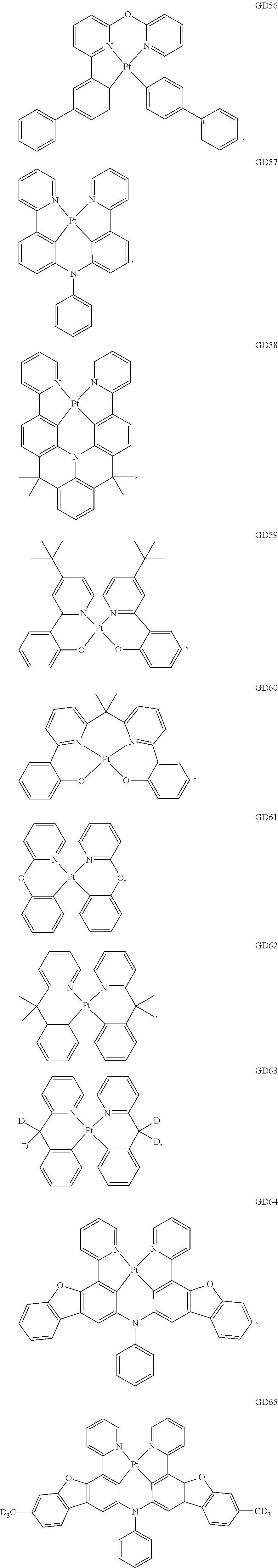

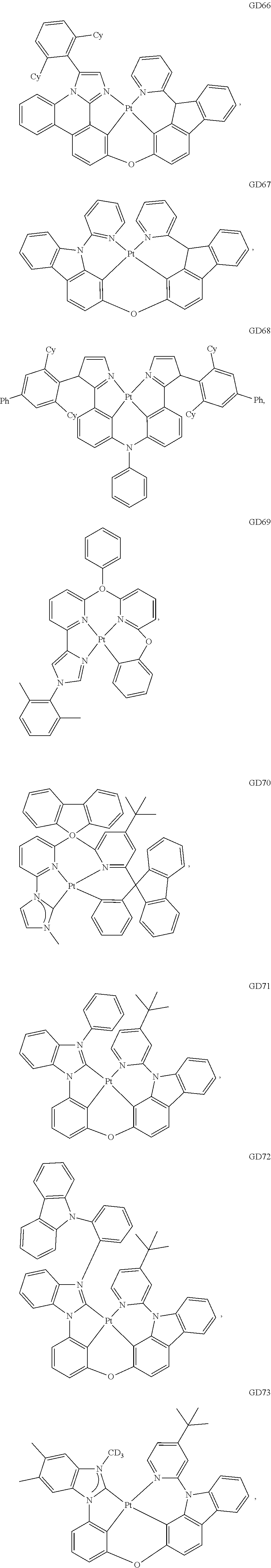

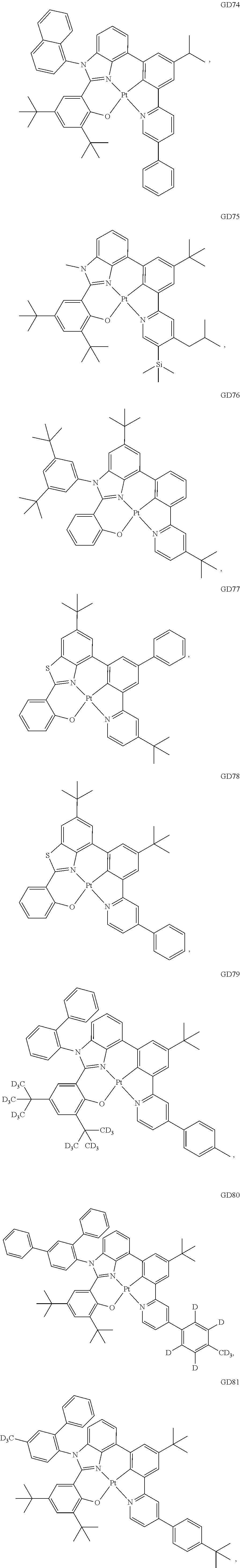

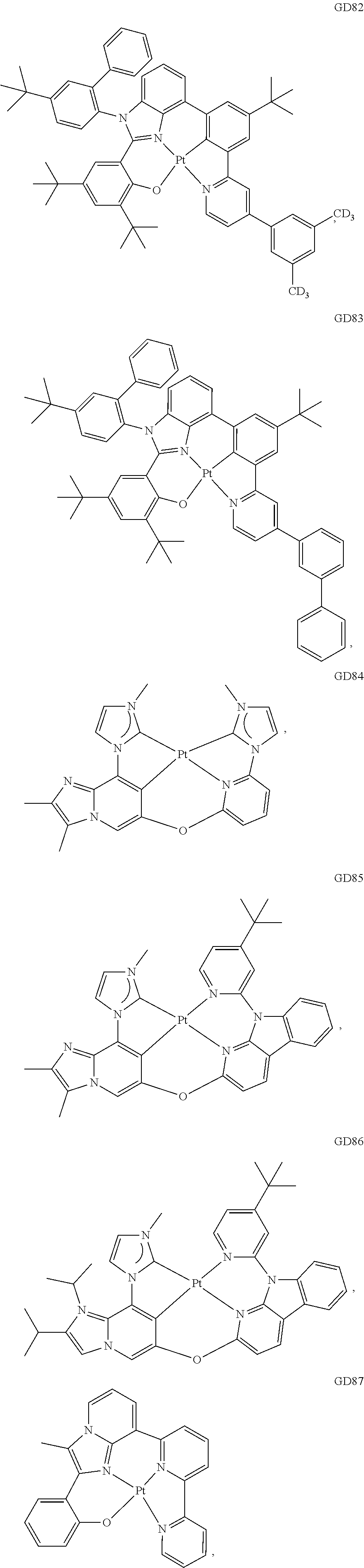

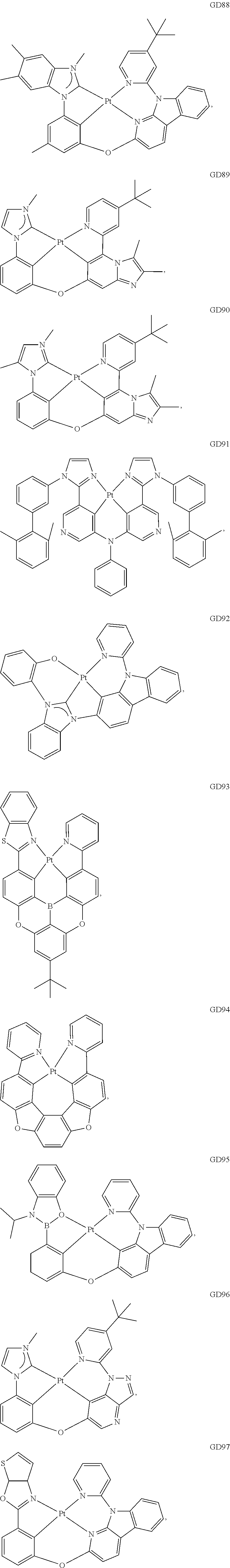

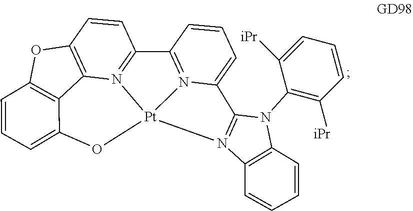

wherein in the above structures, Cy represents cyclohexyl, and ⁱPr represents isopropyl; and

optionally, hydrogens in the structures of Metal Complex GD1 to Metal Complex GD98 can be partially or fully substituted with deuterium.

According to an embodiment of the present disclosure, the emissive layer of the green phosphorescent unit further includes a first compound and a second compound.

According to an embodiment of the present disclosure, the first compound has a structure represented by Formula 12:

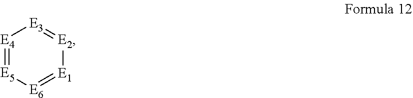

wherein,

E₁ to E₆ are, at each occurrence identically or differently, selected from C, CRₑ₂ or N, at least two of E₁ to E₆ are N, and at least one of E₁ to E₆ is C and joined to Formula 13;

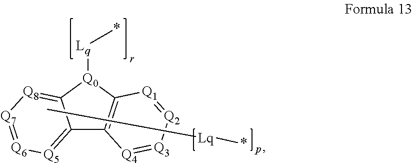

wherein,

Q₀ is, at each occurrence identically or differently, selected from the group consisting of O, S, Se, N, NR_{q0}, CR_{q0}R_{q0}, SiR_{q0}R_{q0}, GeR_{q0}R_{q0} and R_{q0}C═CR_{q0}; when two R_{q0} are present at the same time, the two R_{q0} may be the same or different;

p is 0 or 1; r is 0 or 1;

when Q₀ is selected from N, p is 0, and r is 1;

when Q₀ is selected from the group consisting of O, S, Se, NR_{q0}, CR_{q0}R_{q0}, SiR_{q0}R_{q0}, GeR_{q0}R_{q0} and R_{q0}C═CR_{q0}, p is 1, and r is 0;

L_{q} is, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 20 carbon atoms, substituted or unsubstituted heteroarylene having 3 to 20 carbon atoms or combinations thereof;

Q₁ to Q₈ are, at each occurrence identically or differently, selected from C, CR_{q1} or N;

“ [symbol] ” represents a position where Formula 13 is joined to Formula 12;

Rₑ₂, R_{q0} and R_{q1} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and

adjacent substituents Rₑ₂, R_{q0} and R_{q1} can be optionally joined to form a ring.

In this embodiment, the expression that adjacent substituents Rₑ₂, R_{q0} and R_{q1} can be optionally joined to form a ring is intended to mean that any one or more of groups of adjacent substituents, such as two substituents Rₑ₂, two substituents R_{q0}, and two substituents R_{q1}, can be joined to form a ring. Obviously, it is also possible that none of these groups of adjacent substituents are joined to form a ring.

According to an embodiment of the present disclosure, wherein the first compound is selected from the group consisting of the following:

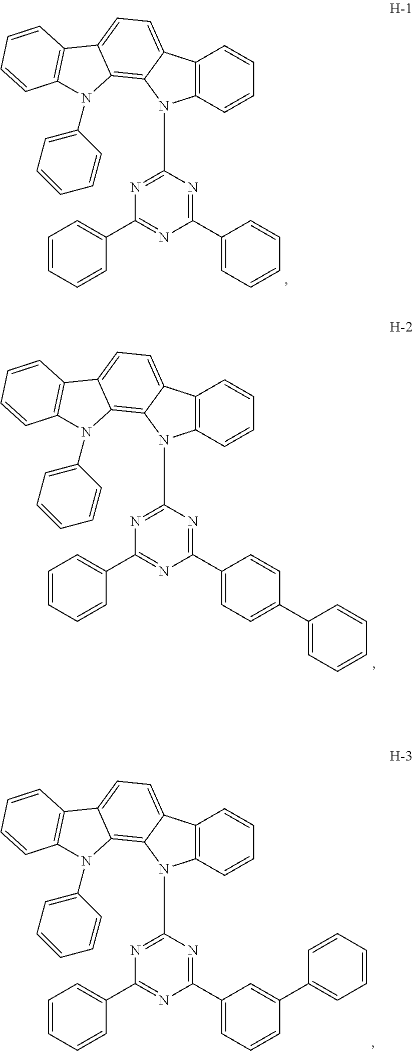 [SMILES: c1ccc(-c2nc(-c3ccccc3)nc(-n3c4ccccc4c4ccc5c6ccccc6n(-c6ccccc6)c5c43)n2)cc1 ; c1ccc(-c2ccc(-c3nc(-c4ccccc4)nc(-n4c5ccccc5c5ccc6c7ccccc7n(-c7ccccc7)c6c54)n3)cc2)cc1 ; c1ccc(-c2cccc(-c3nc(-c4ccccc4)nc(-n4c5ccccc5c5ccc6c7ccccc7n(-c7ccccc7)c6c54)n3)c2)cc1]

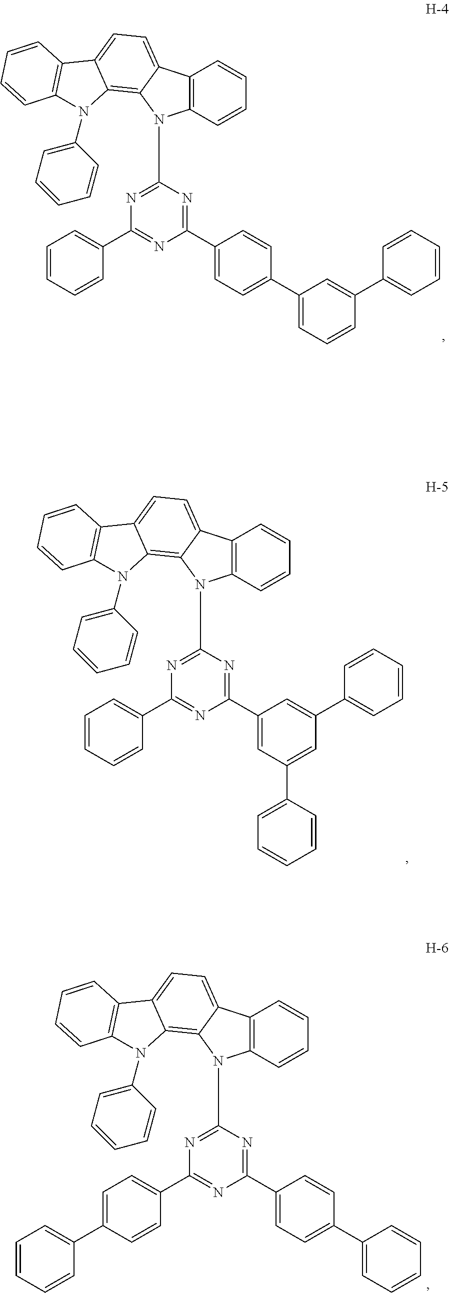 [SMILES: c1ccc(-c2cccc(-c3ccc(-c4nc(-c5ccccc5)nc(-n5c6ccccc6c6ccc7c8ccccc8n(-c8ccccc8)c7c65)n4)cc3)c2)cc1 ; c1ccc(-c2cc(-c3ccccc3)cc(-c3nc(-c4ccccc4)nc(-n4c5ccccc5c5ccc6c7ccccc7n(-c7ccccc7)c6c54)n3)c2)cc1 ; c1ccc(-c2ccc(-c3nc(-c4ccc(-c5ccccc5)cc4)nc(-n4c5ccccc5c5ccc6c7ccccc7n(-c7ccccc7)c6c54)n3)cc2)cc1]

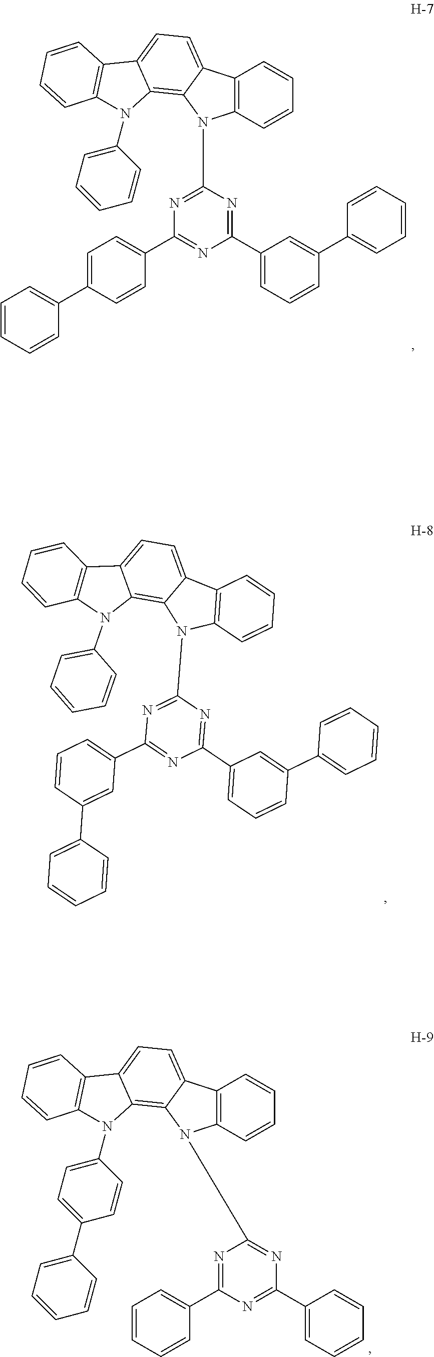 [SMILES: c1ccc(-c2ccc(-c3nc(-c4cccc(-c5ccccc5)c4)nc(-n4c5ccccc5c5ccc6c7ccccc7n(-c7ccccc7)c6c54)n3)cc2)cc1 ; c1ccc(-c2cccc(-c3nc(-c4cccc(-c5ccccc5)c4)nc(-n4c5ccccc5c5ccc6c7ccccc7n(-c7ccccc7)c6c54)n3)c2)cc1 ; c1ccc(-c2ccc(-n3c4ccccc4c4ccc5c6ccccc6n(-c6nc(-c7ccccc7)nc(-c7ccccc7)n6)c5c43)cc2)cc1]

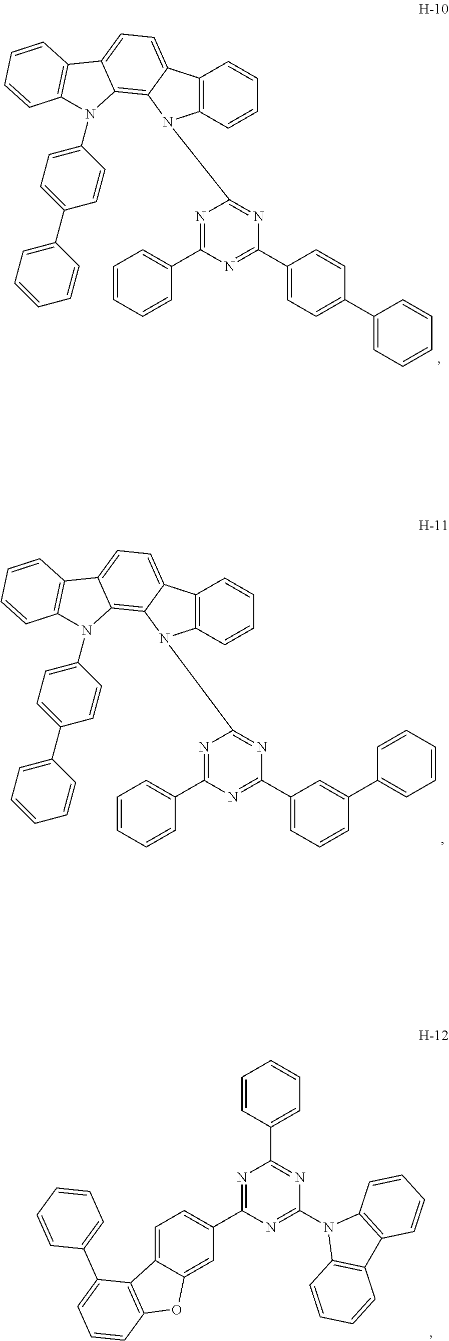 [SMILES: c1ccc(-c2ccc(-c3nc(-c4ccccc4)nc(-n4c5ccccc5c5ccc6c7ccccc7n(-c7ccc(-c8ccccc8)cc7)c6c54)n3)cc2)cc1 ; c1ccc(-c2ccc(-n3c4ccccc4c4ccc5c6ccccc6n(-c6nc(-c7ccccc7)nc(-c7cccc(-c8ccccc8)c7)n6)c5c43)cc2)cc1 ; c1ccc(-c2nc(-c3ccc4c(c3)oc3cccc(-c5ccccc5)c34)nc(-n3c4ccccc4c4ccccc43)n2)cc1]

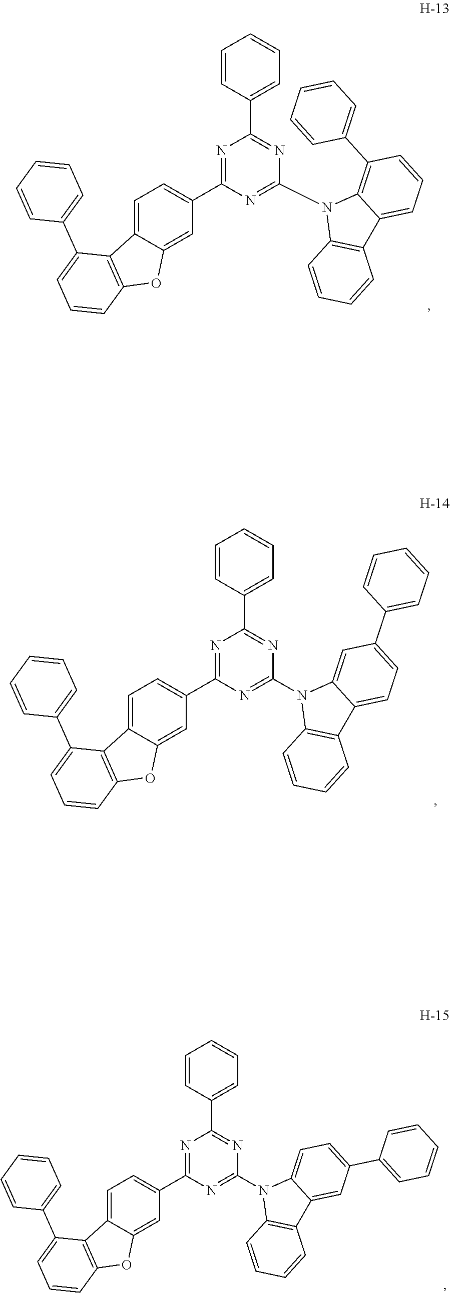 [SMILES: c1ccc(-c2nc(-c3ccc4c(c3)oc3cccc(-c5ccccc5)c34)nc(-n3c4ccccc4c4cccc(-c5ccccc5)c43)n2)cc1 ; c1ccc(-c2ccc3c4ccccc4n(-c4nc(-c5ccccc5)nc(-c5ccc6c(c5)oc5cccc(-c7ccccc7)c56)n4)c3c2)cc1 ; c1ccc(-c2ccc3c(c2)c2ccccc2n3-c2nc(-c3ccccc3)nc(-c3ccc4c(c3)oc3cccc(-c5ccccc5)c34)n2)cc1]

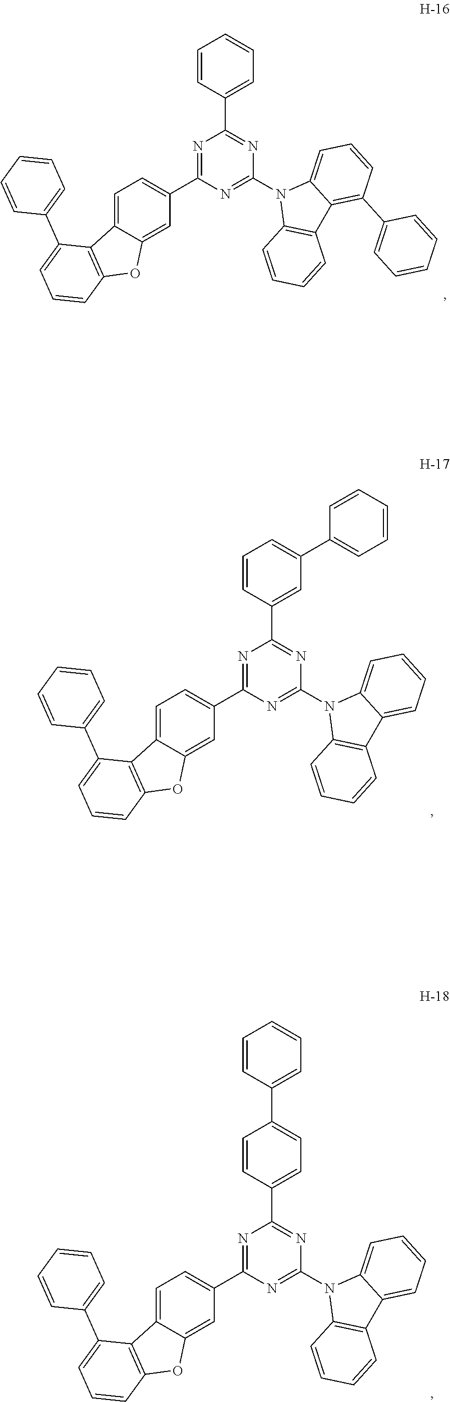 [SMILES: c1ccc(-c2nc(-c3ccc4c(c3)oc3cccc(-c5ccccc5)c34)nc(-n3c4ccccc4c4c(-c5ccccc5)cccc43)n2)cc1 ; c1ccc(-c2cccc(-c3nc(-c4ccc5c(c4)oc4cccc(-c6ccccc6)c45)nc(-n4c5ccccc5c5ccccc54)n3)c2)cc1 ; c1ccc(-c2ccc(-c3nc(-c4ccc5c(c4)oc4cccc(-c6ccccc6)c45)nc(-n4c5ccccc5c5ccccc54)n3)cc2)cc1]

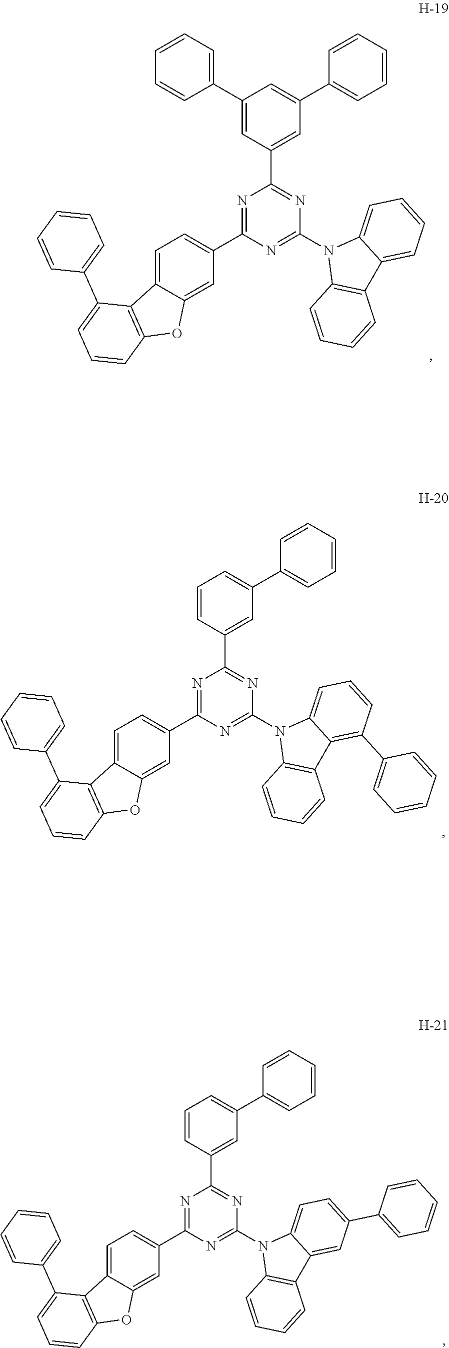 [SMILES: c1ccc(-c2cc(-c3ccccc3)cc(-c3nc(-c4ccc5c(c4)oc4cccc(-c6ccccc6)c45)nc(-n4c5ccccc5c5ccccc54)n3)c2)cc1 ; c1ccc(-c2cccc(-c3nc(-c4ccc5c(c4)oc4cccc(-c6ccccc6)c45)nc(-n4c5ccccc5c5c(-c6ccccc6)cccc54)n3)c2)cc1 ; c1ccc(-c2cccc(-c3nc(-c4ccc5c(c4)oc4cccc(-c6ccccc6)c45)nc(-n4c5ccccc5c5cc(-c6ccccc6)ccc54)n3)c2)cc1]

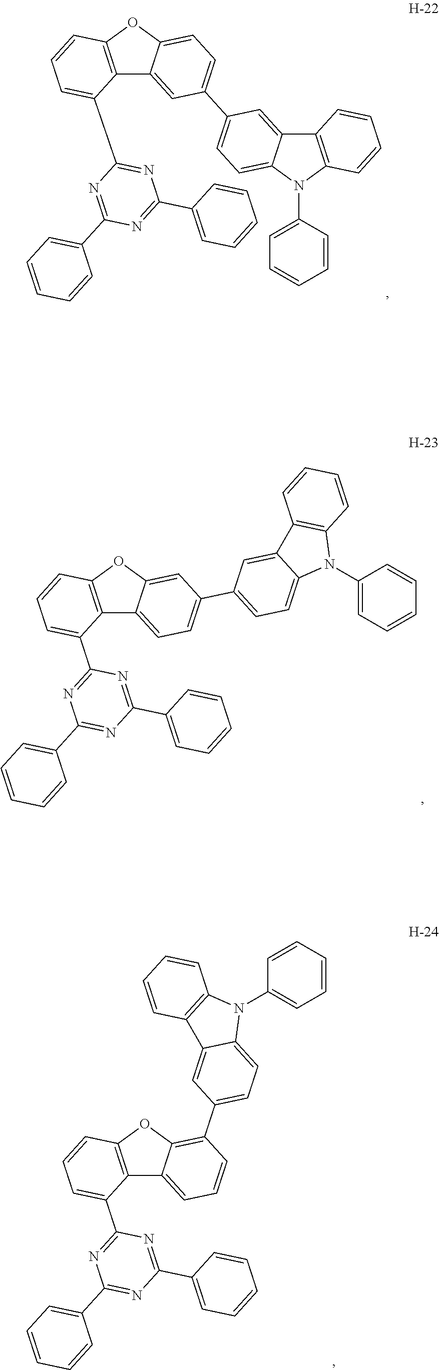 [SMILES: c1ccc(-c2nc(-c3ccccc3)nc(-c3cccc4oc5ccc(-c6ccc7c(c6)c6ccccc6n7-c6ccccc6)cc5c34)n2)cc1 ; c1ccc(-c2nc(-c3ccccc3)nc(-c3cccc4oc5cc(-c6ccc7c(c6)c6ccccc6n7-c6ccccc6)ccc5c34)n2)cc1 ; c1ccc(-c2nc(-c3ccccc3)nc(-c3cccc4oc5c(-c6ccc7c(c6)c6ccccc6n7-c6ccccc6)cccc5c34)n2)cc1]

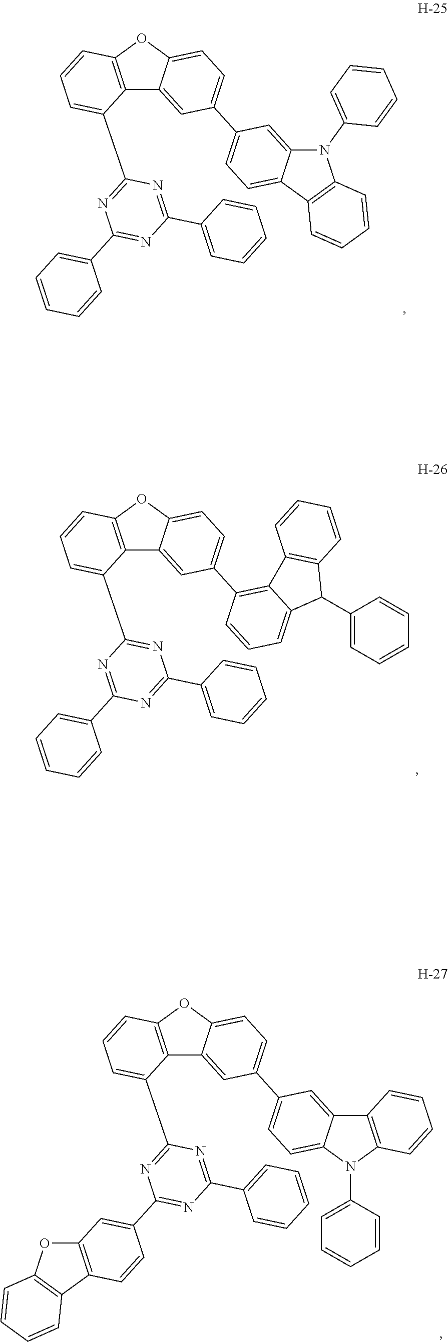 [SMILES: c1ccc(-c2nc(-c3ccccc3)nc(-c3cccc4oc5ccc(-c6ccc7c8ccccc8n(-c8ccccc8)c7c6)cc5c34)n2)cc1 ; c1ccc(-c2nc(-c3ccccc3)nc(-c3cccc4oc5ccc(-c6cccc7c6-c6ccccc6C7c6ccccc6)cc5c34)n2)cc1 ; c1ccc(-c2nc(-c3ccc4c(c3)oc3ccccc34)nc(-c3cccc4oc5ccc(-c6ccc7c(c6)c6ccccc6n7-c6ccccc6)cc5c34)n2)cc1]

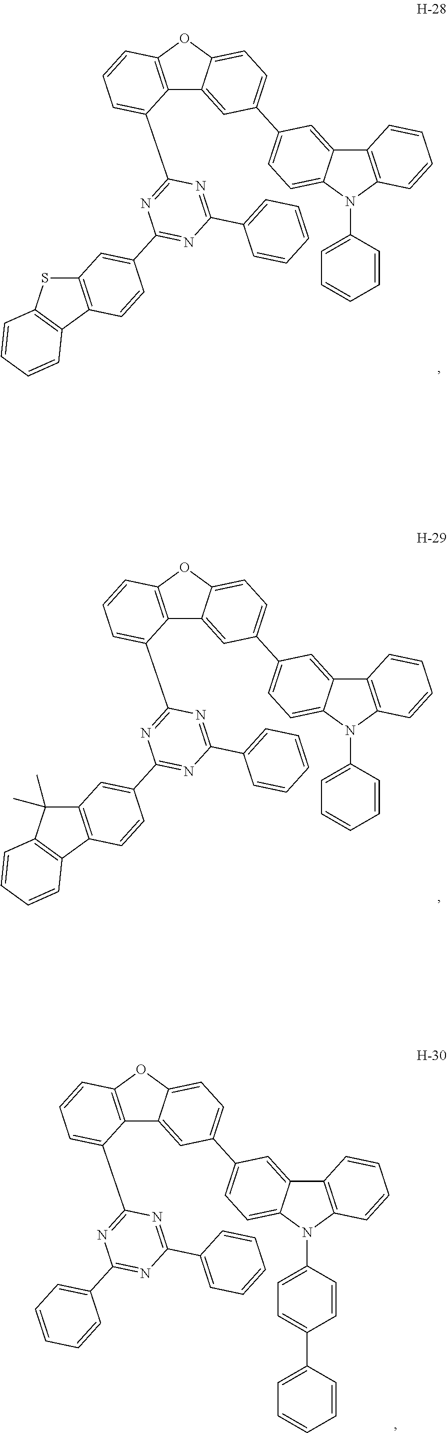 [SMILES: c1ccc(-c2nc(-c3ccc4c(c3)sc3ccccc34)nc(-c3cccc4oc5ccc(-c6ccc7c(c6)c6ccccc6n7-c6ccccc6)cc5c34)n2)cc1 ; CC1(C)c2ccccc2-c2ccc(-c3nc(-c4ccccc4)nc(-c4cccc5oc6ccc(-c7ccc8c(c7)c7ccccc7n8-c7ccccc7)cc6c45)n3)cc21 ; c1ccc(-c2ccc(-n3c4ccccc4c4cc(-c5ccc6oc7cccc(-c8nc(-c9ccccc9)nc(-c9ccccc9)n8)c7c6c5)ccc43)cc2)cc1]

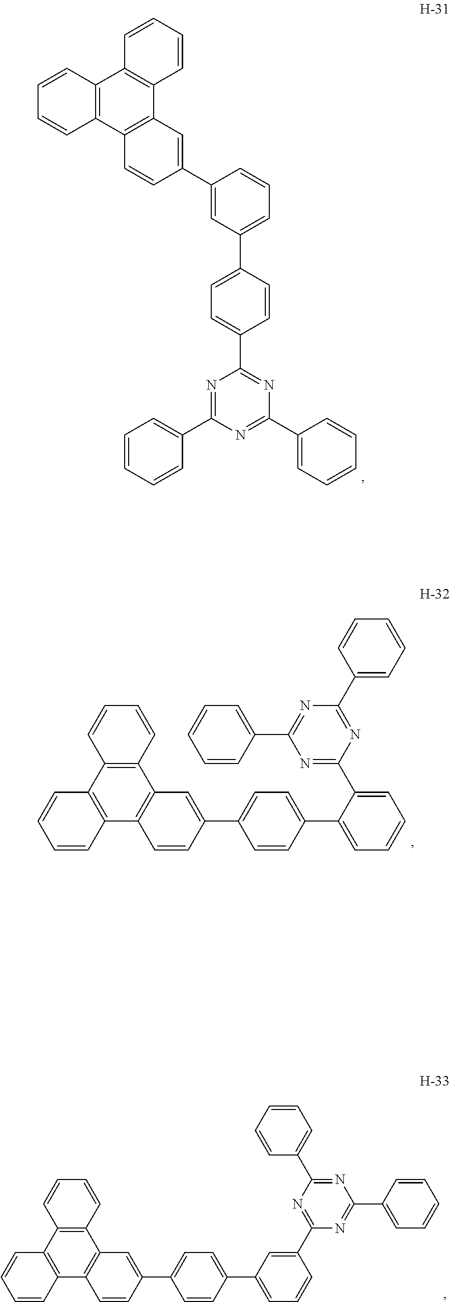 [SMILES: c1ccc(-c2nc(-c3ccccc3)nc(-c3ccc(-c4cccc(-c5ccc6c7ccccc7c7ccccc7c6c5)c4)cc3)n2)cc1 ; c1ccc(-c2nc(-c3ccccc3)nc(-c3ccccc3-c3ccc(-c4ccc5c6ccccc6c6ccccc6c5c4)cc3)n2)cc1 ; c1ccc(-c2nc(-c3ccccc3)nc(-c3cccc(-c4ccc(-c5ccc6c7ccccc7c7ccccc7c6c5)cc4)c3)n2)cc1]

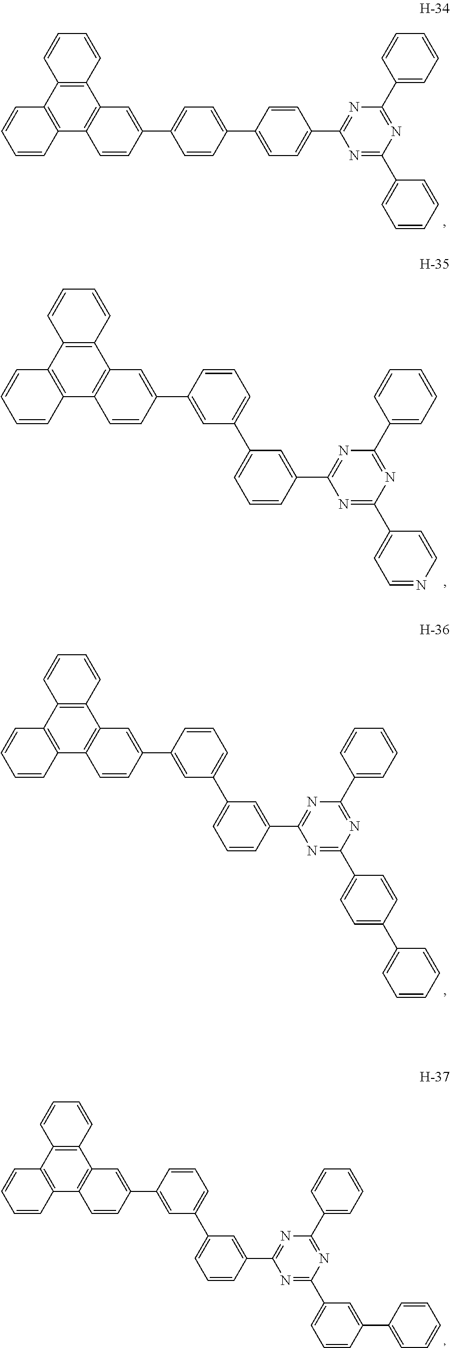 [SMILES: c1ccc(-c2nc(-c3ccccc3)nc(-c3ccc(-c4ccc(-c5ccc6c7ccccc7c7ccccc7c6c5)cc4)cc3)n2)cc1 ; c1ccc(-c2nc(-c3ccncc3)nc(-c3cccc(-c4cccc(-c5ccc6c7ccccc7c7ccccc7c6c5)c4)c3)n2)cc1 ; c1ccc(-c2ccc(-c3nc(-c4ccccc4)nc(-c4cccc(-c5cccc(-c6ccc7c8ccccc8c8ccccc8c7c6)c5)c4)n3)cc2)cc1 ; c1ccc(-c2cccc(-c3nc(-c4ccccc4)nc(-c4cccc(-c5cccc(-c6ccc7c8ccccc8c8ccccc8c7c6)c5)c4)n3)c2)cc1]

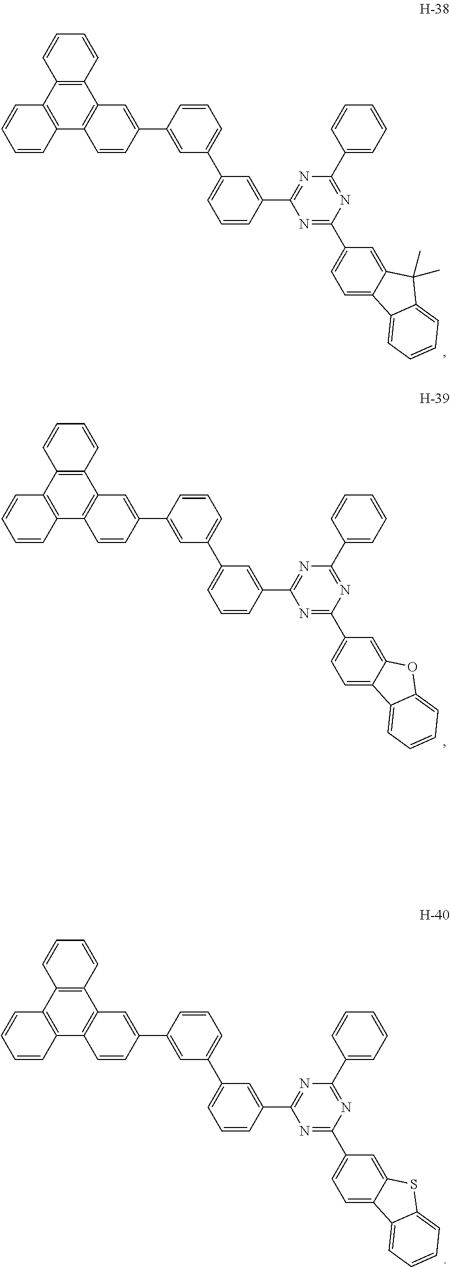 [SMILES: CC1(C)c2ccccc2-c2ccc(-c3nc(-c4ccccc4)nc(-c4cccc(-c5cccc(-c6ccc7c8ccccc8c8ccccc8c7c6)c5)c4)n3)cc21 ; c1ccc(-c2nc(-c3cccc(-c4cccc(-c5ccc6c7ccccc7c7ccccc7c6c5)c4)c3)nc(-c3ccc4c(c3)oc3ccccc34)n2)cc1 ; c1ccc(-c2nc(-c3cccc(-c4cccc(-c5ccc6c7ccccc7c7ccccc7c6c5)c4)c3)nc(-c3ccc4c(c3)sc3ccccc34)n2)cc1]

According to an embodiment of the present disclosure, the second compound has a structure represented by Formula 14:

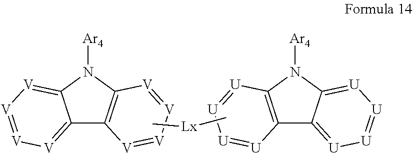

wherein,

Lₓ is, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 20 carbon atoms, substituted or unsubstituted heteroarylene having 3 to 20 carbon atoms or combinations thereof;

V is, at each occurrence identically or differently, selected from C, CRᵥ₁ or N, and at least one of V is C and joined to Lₓ;

U is, at each occurrence identically or differently, selected from C, CRᵤ₁ or N, and at least one of U is C and joined to Lₓ;

Rᵥ₁ and Rᵤ₁ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;

Ar₄ is, at each occurrence identically or differently, selected from substituted or unsubstituted aryl having 6 to 30 carbon atoms, substituted or unsubstituted heteroaryl having 3 to 30 carbon atoms or a combination thereof; and

adjacent substituents Rᵥ₁ and Rᵤ₁ can be optionally joined to form a ring.

In this embodiment, the expression that adjacent substituents Rᵥ₁ and Rᵤ₁ can be optionally joined to form a ring is intended to mean that any one or more of groups of adjacent substituents, such as two substituents Rᵥ₁, and two substituents Rᵤ₁, can be joined to form a ring. Obviously, it is also possible that none of these groups of adjacent substituents are joined to form a ring.

According to an embodiment of the present disclosure, wherein the second compound is selected from the group consisting of the following:

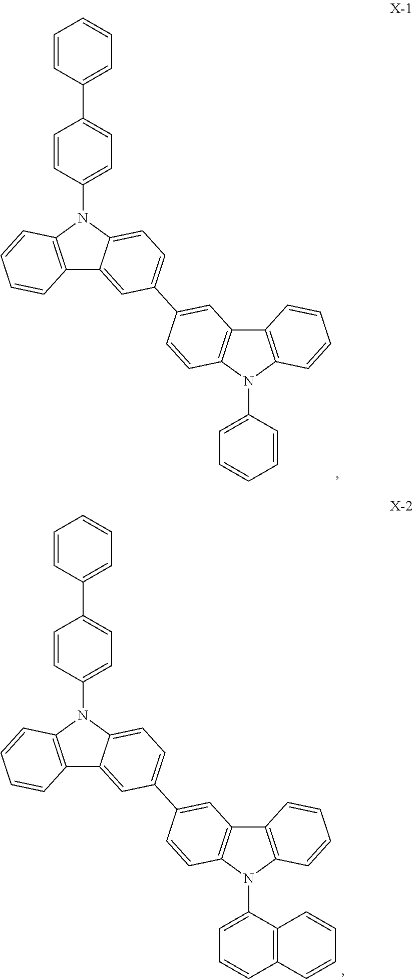 [SMILES: c1ccc(-c2ccc(-n3c4ccccc4c4cc(-c5ccc6c(c5)c5ccccc5n6-c5ccccc5)ccc43)cc2)cc1 ; c1ccc(-c2ccc(-n3c4ccccc4c4cc(-c5ccc6c(c5)c5ccccc5n6-c5cccc6ccccc56)ccc43)cc2)cc1]

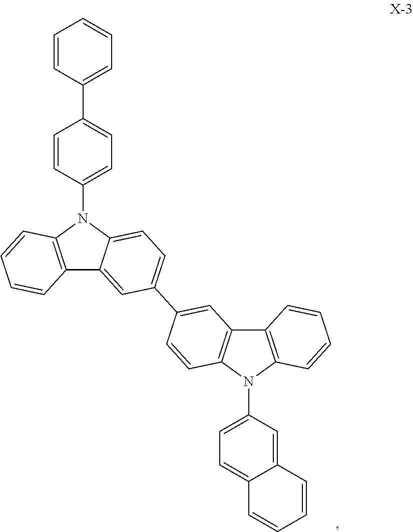 [SMILES: c1ccc(-c2ccc(-n3c4ccccc4c4cc(-c5ccc6c(c5)c5ccccc5n6-c5ccc6ccccc6c5)ccc43)cc2)cc1]

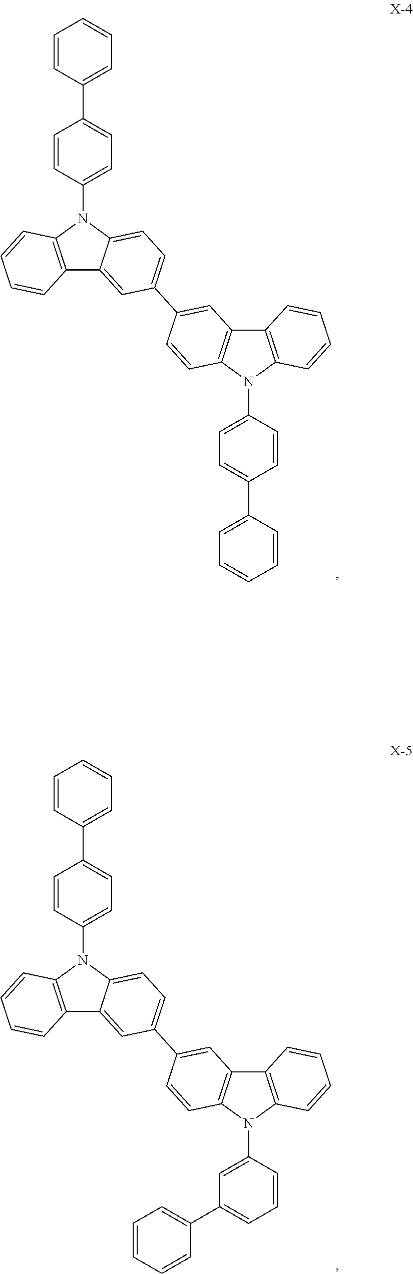 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-n3c4ccccc4c4cc(-c5ccc6c(c5)c5ccccc5n6-c5cccc(-c6ccccc6)c5)ccc43)cc2)cc1]

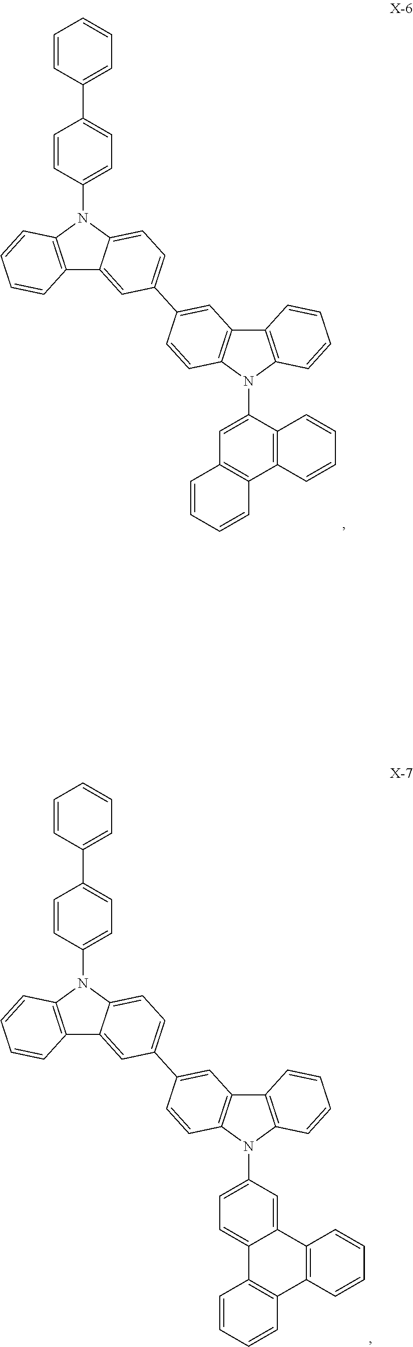 [SMILES: c1ccc(-c2ccc(-n3c4ccccc4c4cc(-c5ccc6c(c5)c5ccccc5n6-c5cc6ccccc6c6ccccc56)ccc43)cc2)cc1 ; c1ccc(-c2ccc(-n3c4ccccc4c4cc(-c5ccc6c(c5)c5ccccc5n6-c5ccc6c7ccccc7c7ccccc7c6c5)ccc43)cc2)cc1]

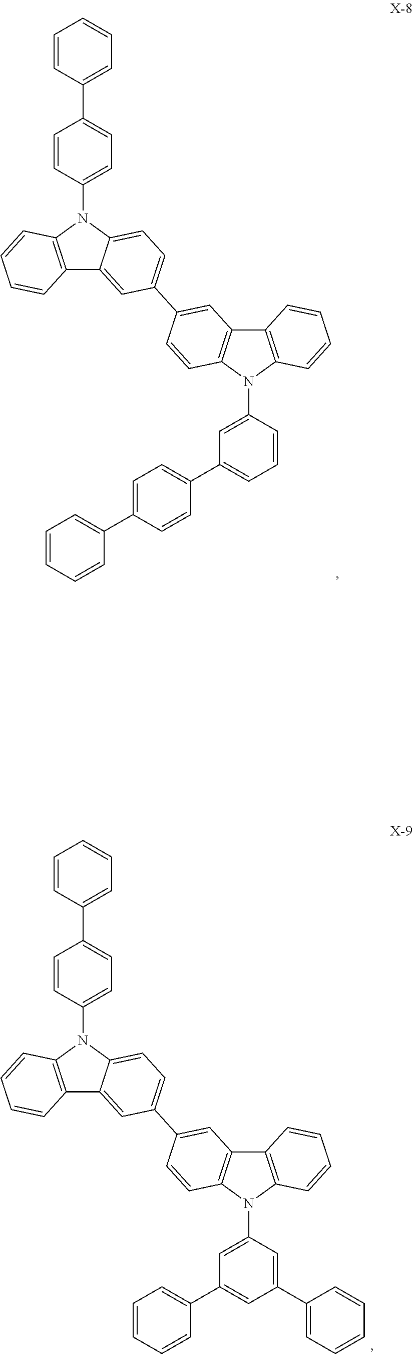 [SMILES: c1ccc(-c2ccc(-c3cccc(-n4c5ccccc5c5cc(-c6ccc7c(c6)c6ccccc6n7-c6ccc(-c7ccccc7)cc6)ccc54)c3)cc2)cc1 ; c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1]

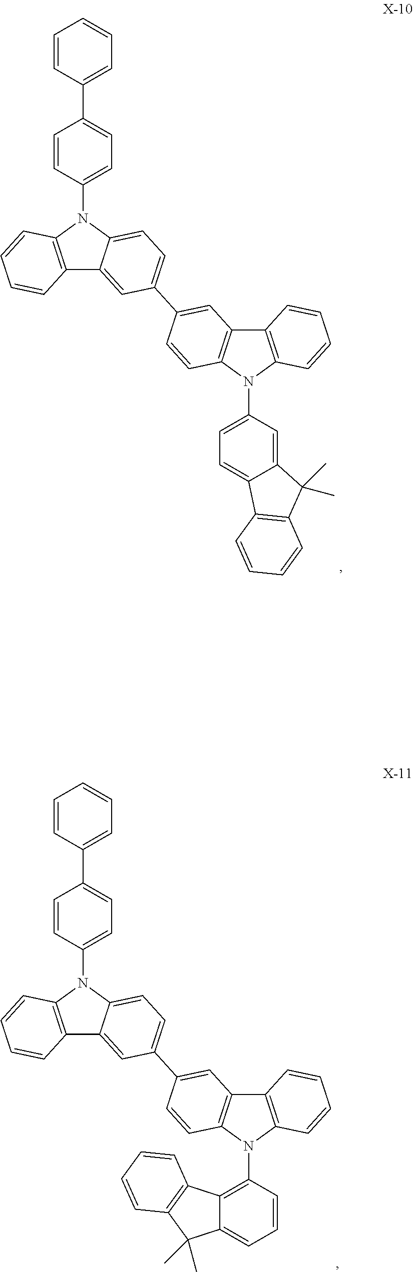 [SMILES: CC1(C)c2ccccc2-c2ccc(-n3c4ccccc4c4cc(-c5ccc6c(c5)c5ccccc5n6-c5ccc(-c6ccccc6)cc5)ccc43)cc21 ; CC1(C)c2ccccc2-c2c(-n3c4ccccc4c4cc(-c5ccc6c(c5)c5ccccc5n6-c5ccc(-c6ccccc6)cc5)ccc43)cccc21]

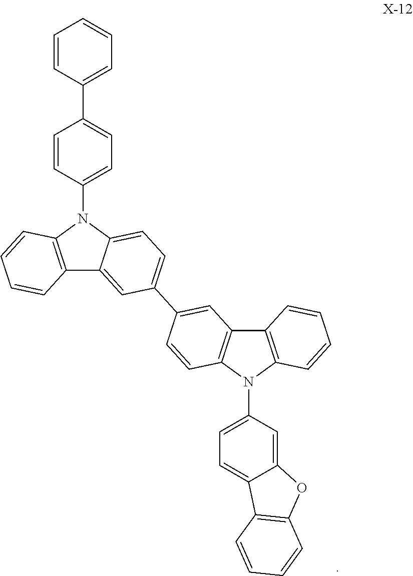 [SMILES: c1ccc(-c2ccc(-n3c4ccccc4c4cc(-c5ccc6c(c5)c5ccccc5n6-c5ccc6c(c5)oc5ccccc56)ccc43)cc2)cc1]

According to an embodiment of the present disclosure, the red phosphorescent material has a general formula of M(Lₐ₂)ₙ(L_{b2})ₖ(L_{c2})ₛ;

wherein M is selected from a metal with a relative atomic mass greater than 40;

Lₐ₂, L_{b2} and L_{c2} are a first ligand, a second ligand and a third ligand coordinated to M, respectively; Lₐ₂, L_{b2} and L_{c2} can be optionally joined to form a multidentate ligand; for example, any two of Lₐ₂, L_{b2} and L_{c2} may be joined to form a tetradentate ligand; in another example, Lₐ₂, L_{b2} and Lₑ₂ may be joined to each other to form a hexadentate ligand; in another example, none of Lₐ₂, L_{b2} and Lₑ₂ are joined so that the multidentate ligand is not formed;

Lₐ₂, L_{b2} and Lₑ₂ may be the same or different; n is 1, 2 or 3; k is 0, 1 or 2; s is 0 or 1; a sum of n, k and s is equal to an oxidation state of M; when n is greater than or equal to 2, multiple Lₐ₂ may the same or different; when k is 2, two L_{b2} may be the same or different;

Lₐ₂ has a structure represented by Formula 15:

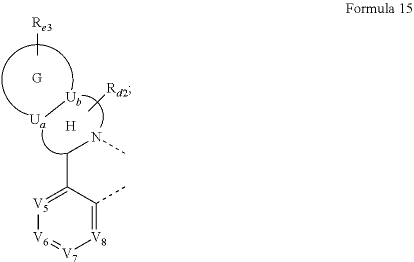

wherein,

the ring H is selected from a five-membered heteroaromatic ring or a six-membered heteroaromatic ring;

the ring G is selected from a five-membered unsaturated carbocyclic ring, a benzene ring, a five-membered heteroaromatic ring or a six-membered heteroaromatic ring;

the ring G and the ring H are fused via Uₐ and U_{b};

Uₐ and U_{b} are, at each occurrence identically or differently, selected from C or N;

R_{d2} and Rₑ₃ represent, at each occurrence identically or differently, mono-substitution, multiple substitutions or non-substitution;

V₅ to V₈ are, at each occurrence identically or differently, selected from CRᵥ₃ or N;

R_{d2}, Rₑ₃ and Rᵥ₃ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;

adjacent substituents R_{d2}, Rₑ₃, Rᵥ₃ can be optionally joined to form a ring;

L_{b2} and L_{c2} are, at each occurrence identically or differently, selected from any one of the following structures:

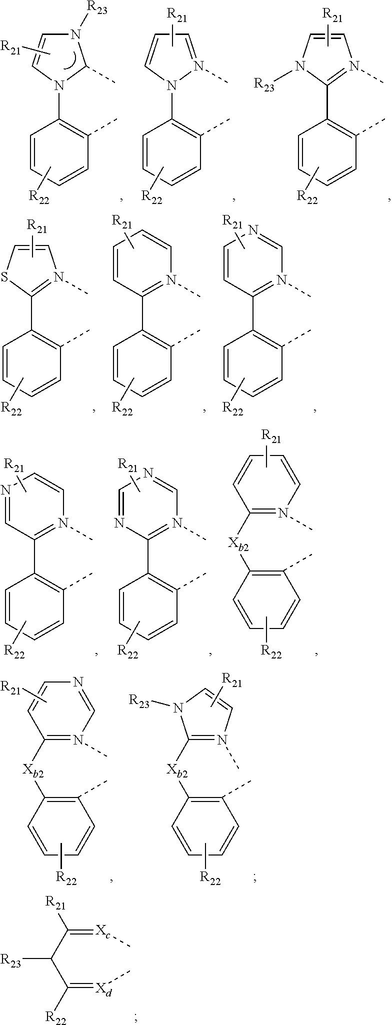

wherein,

R₂₁, R₂₂ and R₂₃ represent, at each occurrence identically or differently, mono-substitution, multiple substitutions or non-substitution;

X_{b2} is, at each occurrence identically or differently, selected from the group consisting of: O, S, Se, NR_{N2} and CR_{C3}R_{C4};

X_{c} and X_{d} are, at each occurrence identically or differently, selected from the group consisting of: O, S, Se and NR_{N3};

R₂₁, R₂₂, R₂₃, R_{N2}, R_{N3}, R_{C3} and R_{C4} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;

and in the structures of the ligands L_{b2} and L_{c2}, adjacent substituents R₂₁, R₂₂, R₂₃, R_{N2}, R_{N3}, R_{C3} and R_{C4} can be optionally joined to form a ring.

In the present disclosure, the expression that adjacent substituents R_{d2}, Rₑ₃, Rᵥ₃ can be optionally joined to form a ring is intended to mean that any one or more of groups of adjacent substituents, such as two substituents R_{d2}, two substituents Rₑ₃, two substituents Rᵥ₃, substituents Rₑ₃ and R_{d2}, and substituents R_{d2} and Rᵥ₃, can be joined to form a ring. Obviously, it is also possible that none of these groups of adjacent substituents are joined to form a ring.

In this embodiment, the expression that adjacent substituents R₂₁, R₂₂, R₂₃, R_{N2}, R_{N3}, R_{C3} and R_{C4} can be optionally joined to form a ring is intended to mean that any one or more of groups of adjacent substituents, such as two substituents R₂₁, two substituents R₂₂, two substituents R₂₃, substituents R₂₁ and R₂₃, substituents R₂₂ and R₂₃, substituents R₂₁ and R_{N2}, substituents R₂₂ and R_{N2}, substituents R₂₃ and R_{N2}, substituents R₂₁ and R_{C3}, substituents R₂₂ and R_{C3}, substituents R₂₃ and R_{C3}, substituents R₂₁ and R_{C4}, substituents R₂₂ and R_{C4}, and substituents R₂₃ and R_{c4}, can be joined to form a ring. Obviously, it is also possible that none of these groups of adjacent substituents are joined to form a ring.

According to an embodiment of the present disclosure, the red phosphorescent material has a general formula of M(Lₐ₂)ₙ(L_{b2})ₖ;

wherein M is selected from a metal with a relative atomic mass greater than 40;

Lₐ₂ and L_{b2} are a first ligand and a second ligand coordinated to M, respectively; Lₐ₂ and L_{b2} can be optionally joined to form a multidentate ligand;

n is 1, 2 or 3; k is 0, 1 or 2; a sum of m and k is equal to an oxidation state of M; when n is greater than or equal to 2, multiple Lₐ₂ may be the same or different; when k is 2, two L_{b2} may be the same or different;

Lₐ₂ has a structure represented by Formula 16:

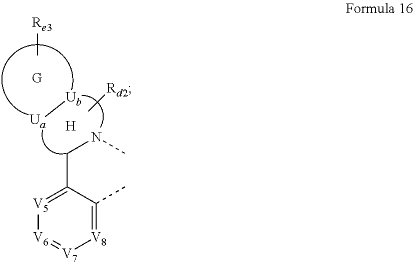

wherein,

the ring H is selected from a five-membered heteroaromatic ring or a six-membered heteroaromatic ring;

the ring G is selected from a five-membered unsaturated carbocyclic ring, a benzene ring, a five-membered heteroaromatic ring or a six-membered heteroaromatic ring;

the ring G and the ring H are fused via Uₐ and U_{b};

Uₐ and U_{b} are, at each occurrence identically or differently, selected from C or N;

R_{d2} and Rₑ₃ represent, at each occurrence identically or differently, mono-substitution, multiple substitutions or non-substitution;

V₅ to V₈ are, at each occurrence identically or differently, selected from CRᵥ₃ or N;

R_{d2}, Rₑ₃ and Rᵥ₃ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;

adjacent substituents R_{d2}, Rₑ₃, Rᵥ₃ can be optionally joined to form a ring;

the ligand L_{b2} has the following structure:

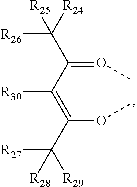

wherein R₂₄ to R₃₀ are each independently selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a sulfanyl group, a sulfinyl group, a sulfonyl group, a phosphino group and combinations thereof.

According to an embodiment of the present disclosure, the ligand L_{b2} has the following structure:

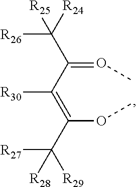

wherein at least one of R₂₄ to R₂₆ is selected from substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms or a combination thereof; and/or at least one of R₂₇ to R₂₉ is selected from substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms or a combination thereof.

According to an embodiment of the present disclosure, the ligand L_{b2} has the following structure:

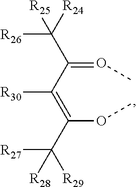

wherein at least two of R₂₄ to R₂₆ are, at each occurrence identically or differently, selected from substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms or a combination thereof; and/or at least two of R₂₇ to R₂₉ are, at each occurrence identically or differently, selected from substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms or a combination thereof.

According to an embodiment of the present disclosure, the ligand L_{b2} has the following structure:

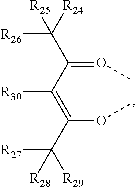

wherein at least two of R₂₄ to R₂₆ are, at each occurrence identically or differently, selected from substituted or unsubstituted alkyl having 2 to 20 carbon atoms, substituted or unsubstituted cycloalkyl having 3 to 20 ring carbon atoms, substituted or unsubstituted heteroalkyl having 2 to 20 carbon atoms or a combination thereof; and/or at least two of R₂₇ to R₂₉ are, at each occurrence identically or differently, selected from substituted or unsubstituted alkyl having 2 to 20 carbon atoms, substituted or unsubstituted cycloalkyl having 3 to 20 ring carbon atoms, substituted or unsubstituted heteroalkyl having 2 to 20 carbon atoms or a combination thereof.

According to an embodiment of the present disclosure, the red phosphorescent material is an Ir complex and has a structure represented by any one of Ir(Lₐ₂)(L_{b2})(L_{c2}), Ir(Lₐ₂)₂(L_{b2}), Ir(Lₐ₂)₂(L_{c2}) or Ir(Lₐ₂)(L_{c2})₂.

According to an embodiment of the present disclosure, the red phosphorescent material is an Ir complex and comprises the ligand Lₐ₂, wherein Lₐ₂ has the structure represented by Formula 15 and at least comprises one structural unit selected from the group consisting of an aromatic ring formed by fusing a six-membered ring to a six-membered ring, a heteroaromatic ring formed by fusing a six-membered ring to a six-membered ring, an aromatic ring formed by fusing a six-membered ring to a five-membered ring and a heteroaromatic ring formed by fusing a six-membered ring to a five-membered ring.

According to an embodiment of the present disclosure, the red phosphorescent material is an Ir complex and comprises the ligand Lₐ₂, wherein Lₐ₂ has the structure represented by Formula 15 and comprises at least one structural unit selected from the group consisting of naphthalene, phenanthrene, quinoline, isoquinoline and azaphenanthrene.

According to an embodiment of the present disclosure, the red phosphorescent material is selected from the group consisting of the following structures:

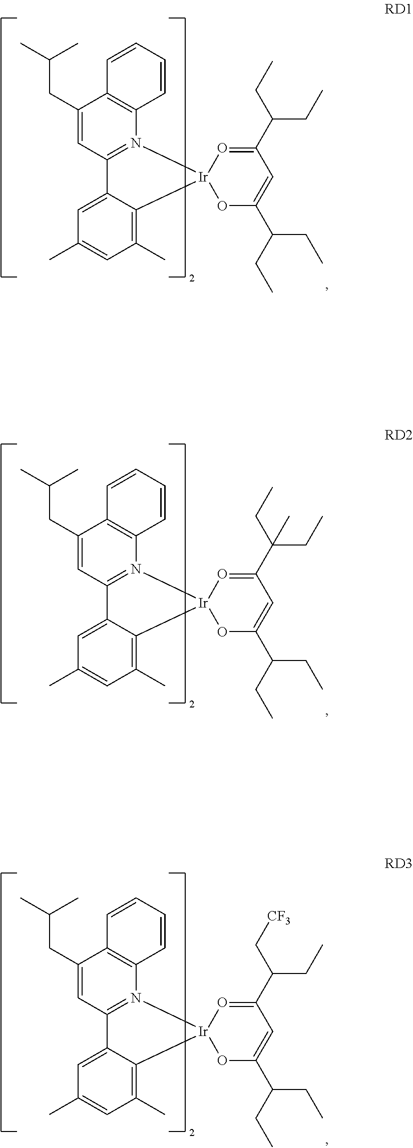

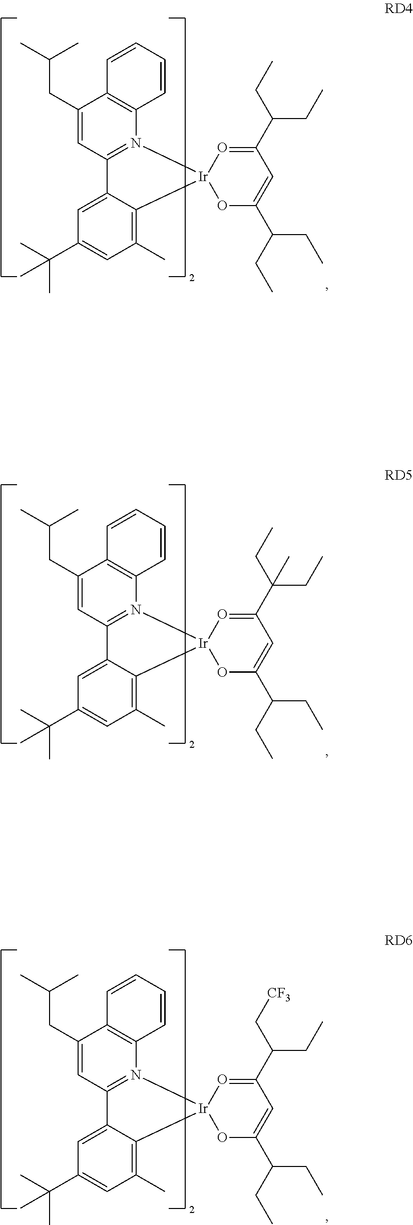

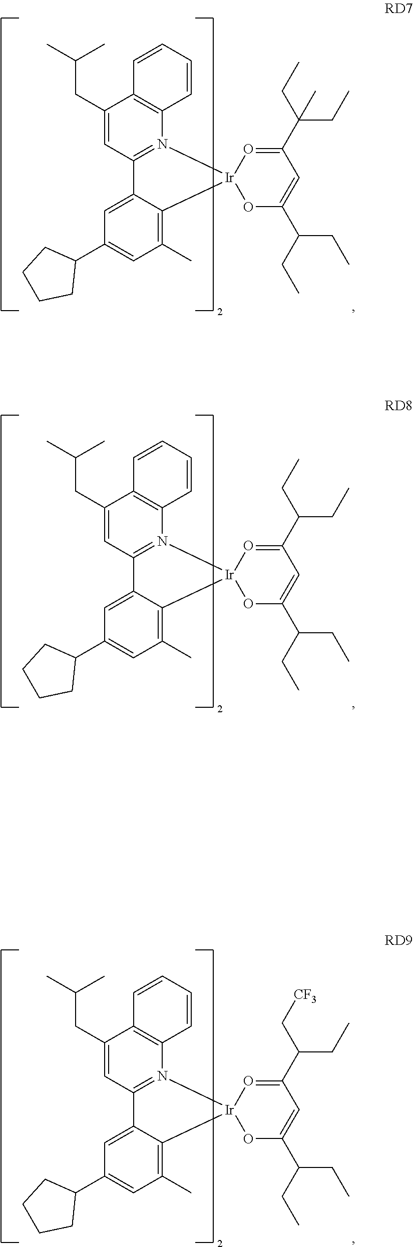

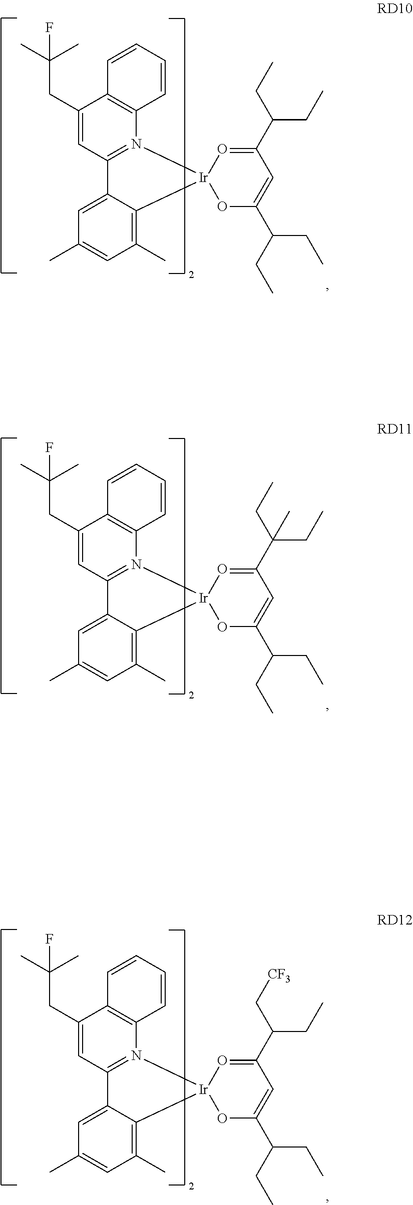

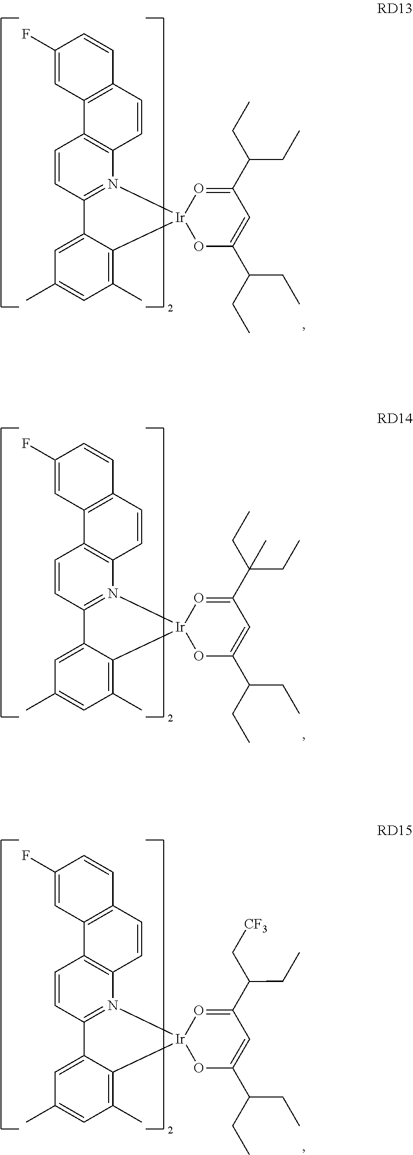

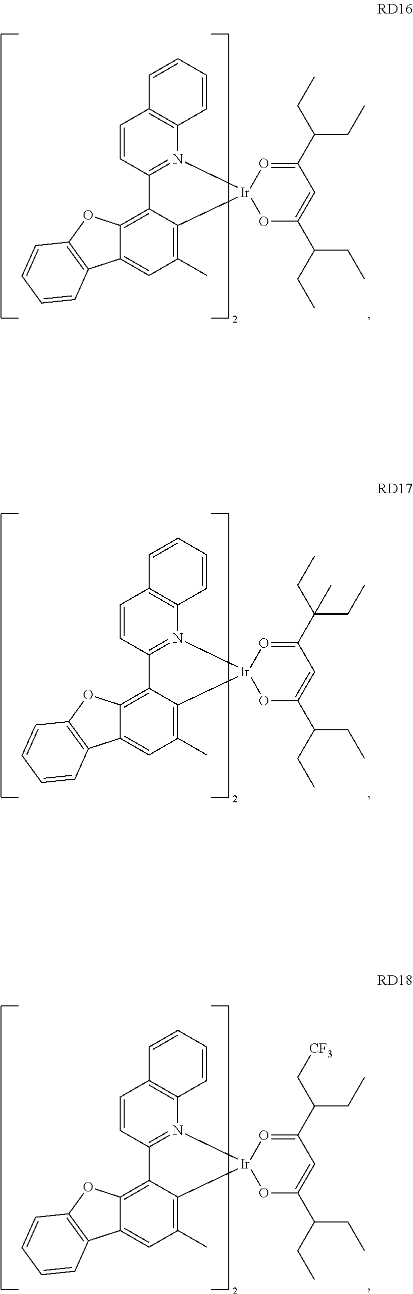

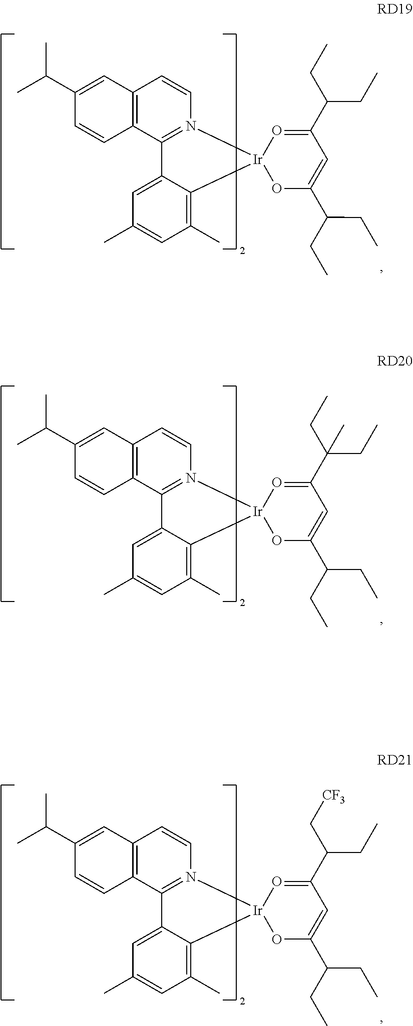

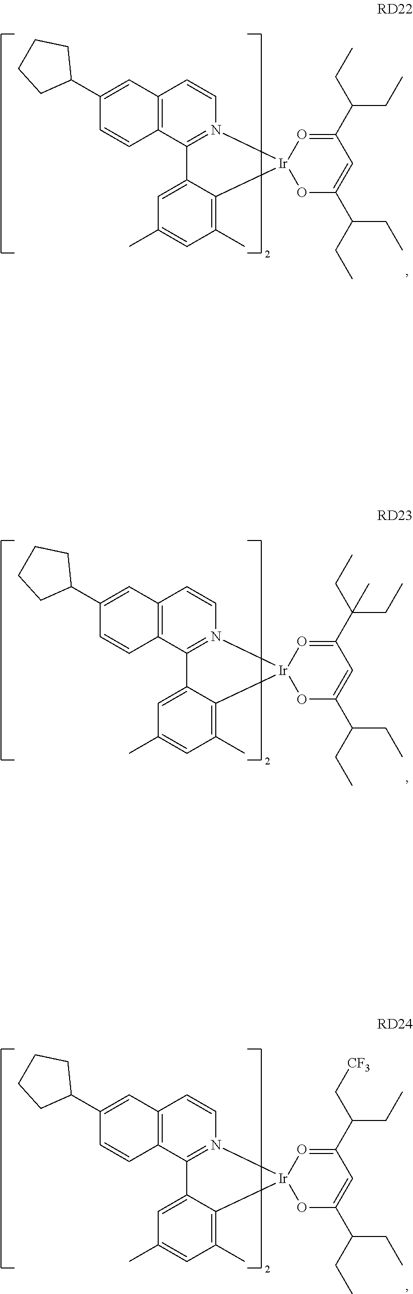

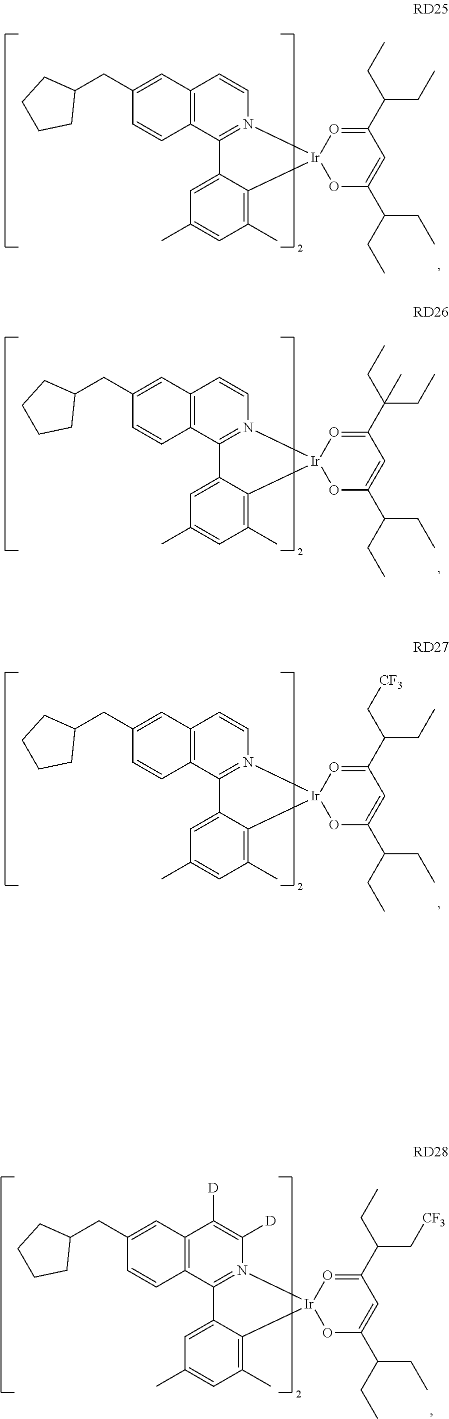

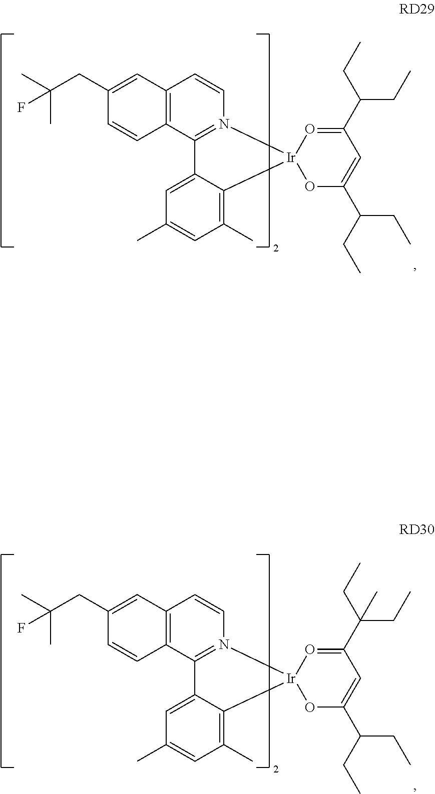

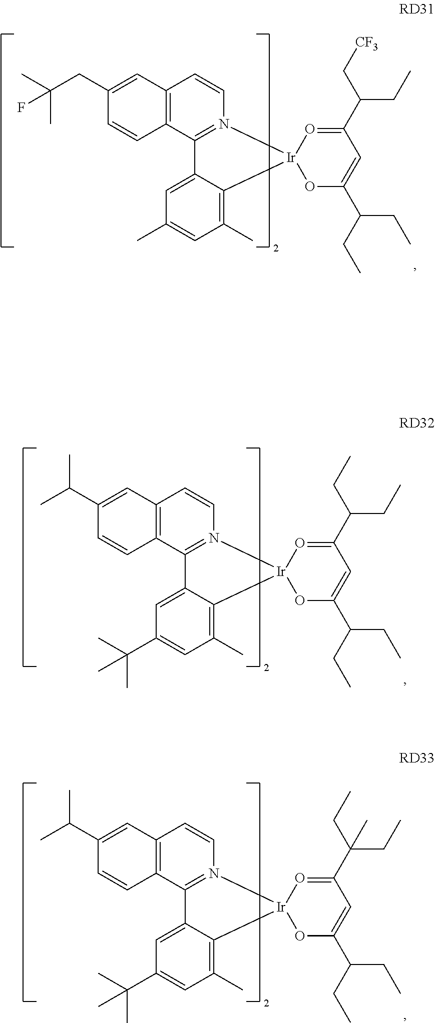

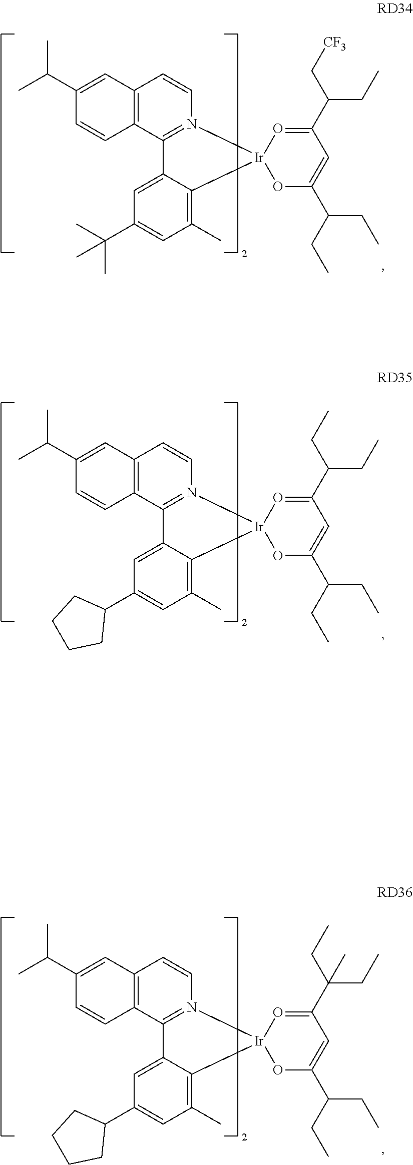

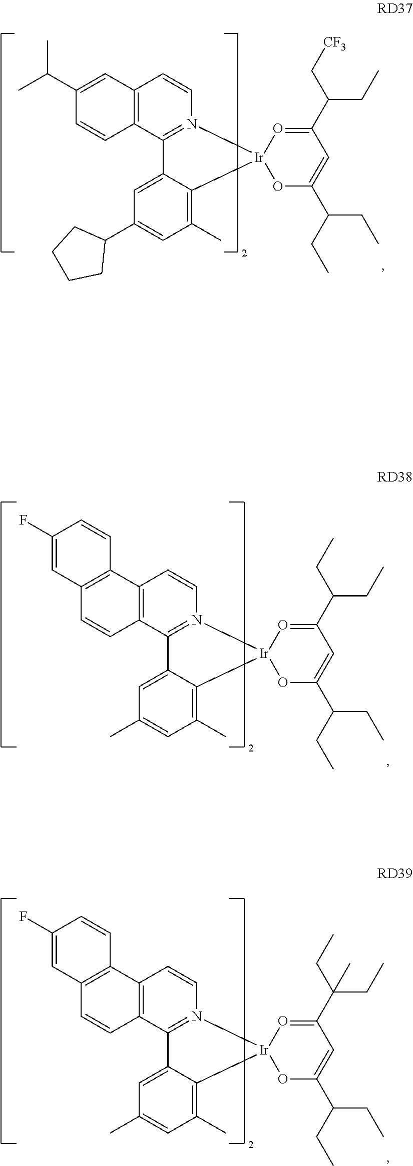

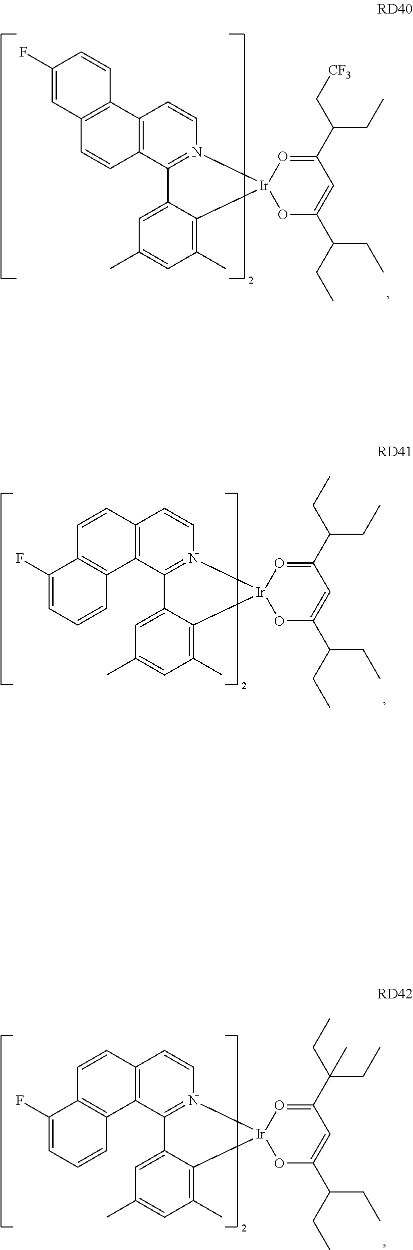

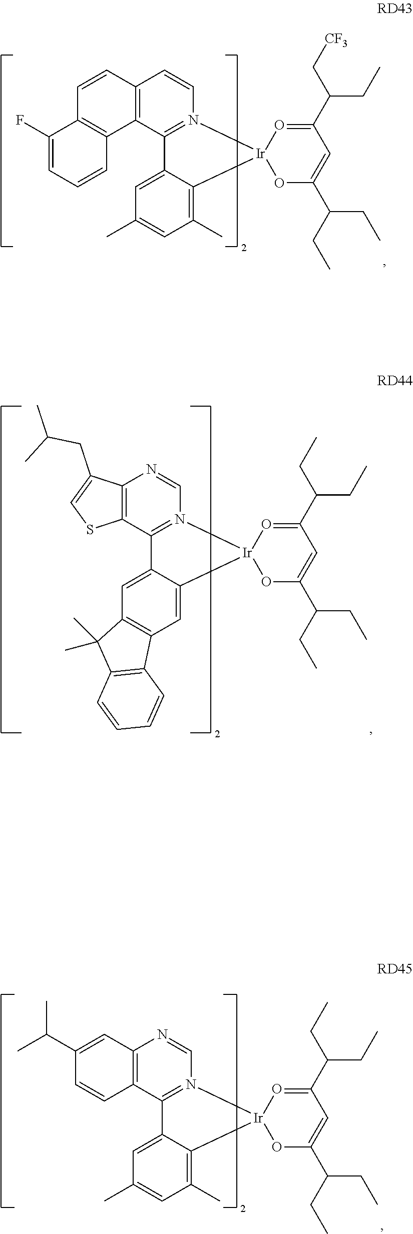

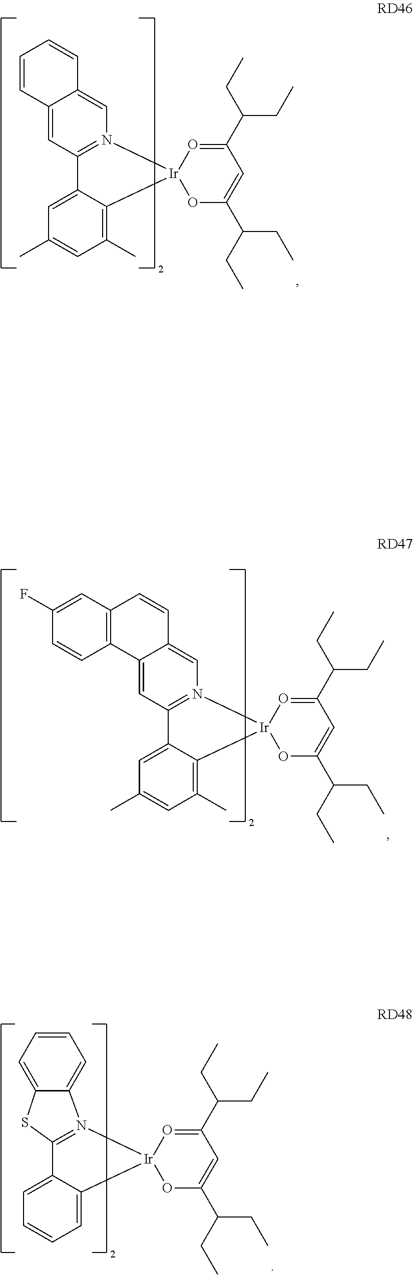

According to an embodiment of the present disclosure, the emissive layer of the red phosphorescent unit further comprises a host material, wherein the host material may be a conventional host material in the related art, for example, may typically include the following host materials without limitation:

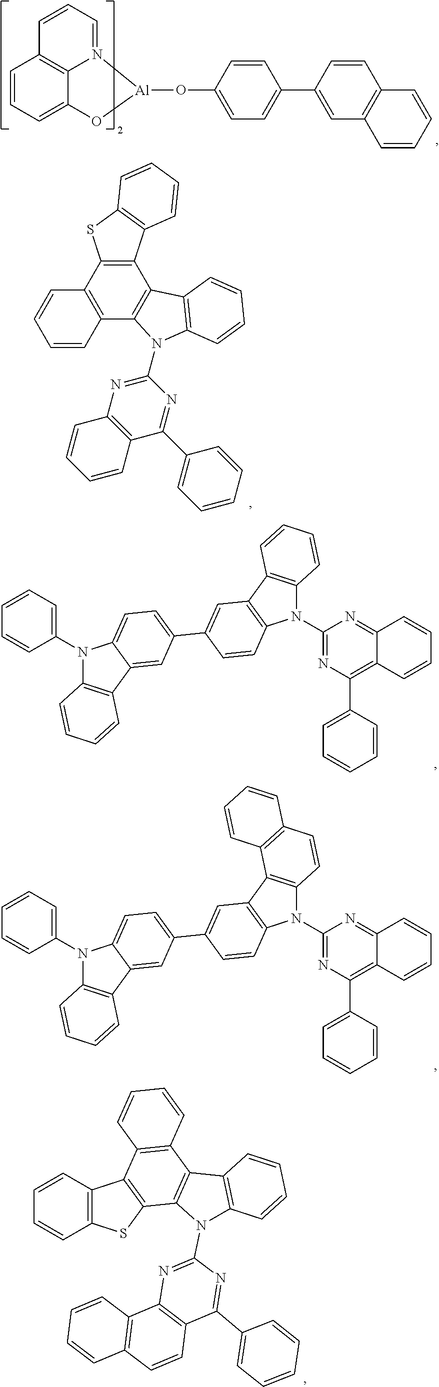

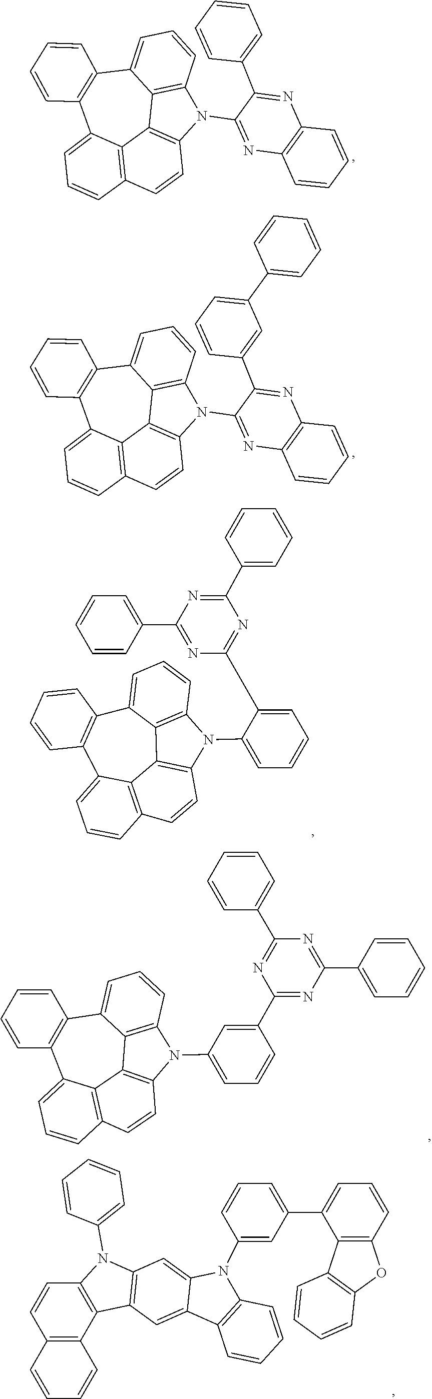

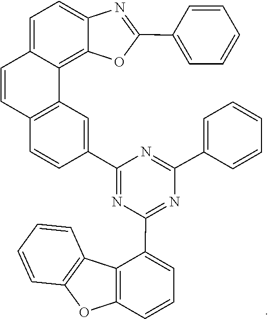 [SMILES: c1ccc(-c2nc(-c3ccc4ccc5ccc6nc(-c7ccccc7)oc6c5c4c3)nc(-c3cccc4oc5ccccc5c34)n2)cc1]

According to an embodiment of the present disclosure, disclosed is a display assembly, which comprises the device described according to any one of the preceding embodiments.

Combination with Other Materials

The materials described in the present disclosure for a particular layer in an organic light emitting device can be used in combination with various other materials present in the device. The combinations of these materials are described in more detail in U.S. Pat. App. No. 20160359122 at paragraphs 0132-0161, which is incorporated by reference herein in its entirety. The materials described or referred to the disclosure are non-limiting examples of materials that may be useful in combination with the compounds disclosed herein, and one of skill in the art can readily consult the literature to identify other materials that may be useful in combination.

The materials described herein as useful for a particular layer in an organic light emitting device may be used in combination with a variety of other materials present in the device. For example, a combination of the first compound and the second compound disclosed herein may be used in combination with a wide variety of emissive dopants, hosts, transporting layers, blocking layers, injection layers, electrodes and other layers that may be present. The combination of these materials is described in detail in paragraphs 0080-0101 of U.S. Pat. App. No. 20150349273, which is incorporated by reference herein in its entirety. The materials described or referred to the disclosure are non-limiting examples of materials that may be useful in combination with the compounds disclosed herein, and one of skill in the art can readily consult the literature to identify other materials that may be useful in combination.

In the embodiments of material synthesis, all reactions were performed under nitrogen protection unless otherwise stated. All reaction solvents were anhydrous and used as received from commercial sources. Synthetic products were structurally confirmed and tested for properties using one or more conventional equipment in the art (including, but not limited to, nuclear magnetic resonance instrument produced by BRUKER, liquid chromatograph produced by SHIMADZU, liquid chromatograph-mass spectrometry produced by SHIMADZU, gas chromatograph-mass spectrometry produced by SHIMADZU, differential Scanning calorimeters produced by SHIMADZU, fluorescence spectrophotometer produced by SHANGHAI LENGGUANG TECH., electrochemical workstation produced by WUHAN CORRTEST, and sublimation apparatus produced by ANHUI BEQ, etc.) by methods well known to the persons skilled in the art. In the embodiments of the device, the characteristics of the device were also tested using conventional equipment in the art (including, but not limited to, evaporator produced by ANGSTROM ENGINEERING, optical testing system produced by SUZHOU FATAR, life testing system produced by SUZHOU FATAR, and ellipsometer produced by BEIJING ELLITOP, etc.) by methods well known to the persons skilled in the art. As the persons skilled in the art are aware of the above-mentioned equipment use, test methods and other related contents, the inherent data of the sample can be obtained with certainty and without influence, so the above related contents are not further described in this present disclosure.

### MATERIAL SYNTHESIS EXAMPLE

The method for preparing a compound in the present disclosure is not limited herein.

Typically, the following compounds are used as examples without limitation, and synthesis routes and preparation methods thereof are described below.

### Synthesis Example 1: Synthesis of Compound HB-3-1

Step 1:

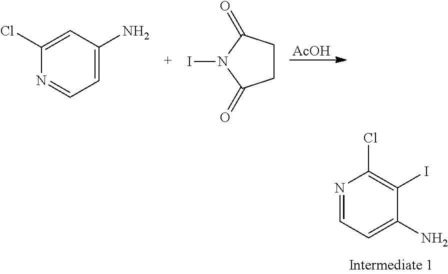 [SMILES: "AcOH" ; Nc1ccnc(Cl)c1 ; O=C1CCC(=O)N1I ; Nc1ccnc(Cl)c1I]

2-Chloro-4-aminopyridine (128 g, 1 mol) and N-iodosuccinimide (224 g, 1 mol) were mixed, and acetic acid (500 mL) was added and heated to 90° C. for reaction. After the reaction was completed, the system was purified through column chromatography to obtain Intermediate 1 (79 g, 311 mmol, 31%).

Step 2:

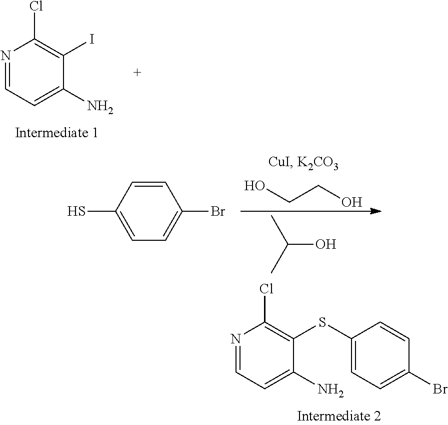

Under nitrogen protection, Intermediate 1 (79 g, 311 mmol), p-bromothiophenol (58 g, 307 mmol), cuprous iodide (3 g, 15 mmol), ethylene glycol (35 mL, 614 mmol) and potassium carbonate (85 g, 614 mmol) were added to a 2 L two-necked flask, and isopropanol (1 L) was added. The system was heated to 90° C. After the reaction was completed, the system was purified through the column chromatography to obtain Intermediate 2 (70 g, 222 mmol, 71%).

Step 3:

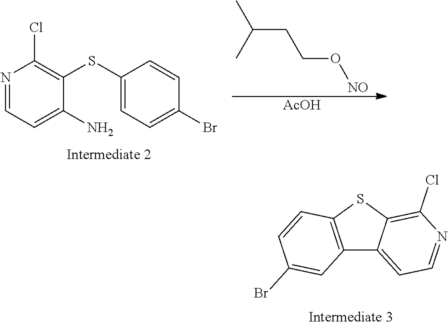

Intermediate 2 (49.5 g, 157 mmol) was dissolved in acetic acid (600 mL), isopentyl nitrite (20 mL, 157 mmol) was added, and the system was reacted at room temperature and stirred for 1 h. Then, isopentyl nitrite (7 mL) was supplemented and reacted for 1 h. After the reaction was completed, the system was purified through the column chromatography to obtain Intermediate 3 (36.8 g, 123 mmol, 78%).

Step 4:

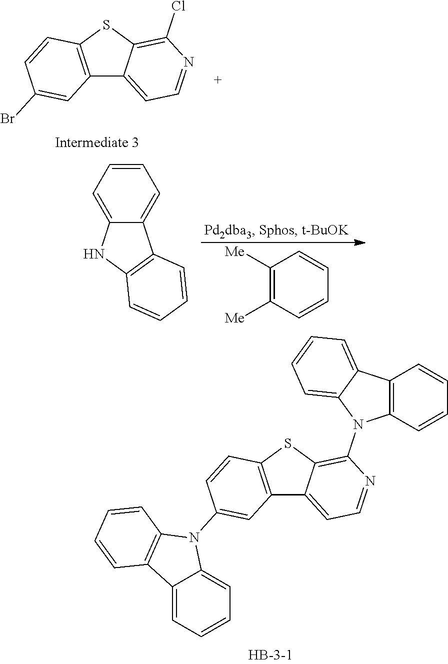

Under nitrogen protection, Intermediate 3 (14.5 g, 50 mmol), carbazole (25 g, 150 mmol), Pd₂dba₃ (4.5 g, 5 mmol), Sphos (8 g, 20 mmol) and t-BuOK (28.8 g, 300 mmol) were mixed, and xylene (1.25 L) was added and heated to 140° C. for reaction. After the reaction was completed, the system was purified through the column chromatography to obtain Compound HB-3-1 (9 g, 17.5 mmol, 35%). The product was identified as the target product with a molecular weight of 515.1.

### Synthesis Comparative Example 1: Synthesis of Compound HB-1

Step 1:

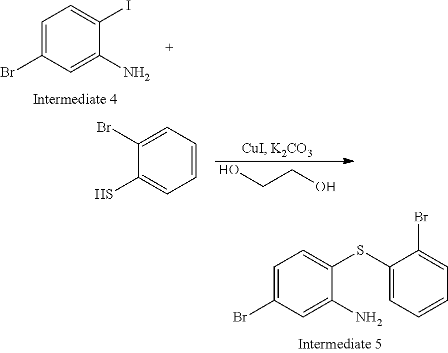

Under nitrogen protection, Intermediate 4 (40 g, 134 mmol), o-bromothiophenol (25 g, 132 mmol), cuprous iodide (1.27 g, 6.68 mmol), ethylene glycol (15 mL, 268 mmol) and potassium carbonate (36.8 g, 266 mmol) were mixed and heated to 110° C. After the reaction was completed, the system was purified through column chromatography to obtain Intermediate 5 (19 g, 52.9 mmol, 39%).

Step 2:

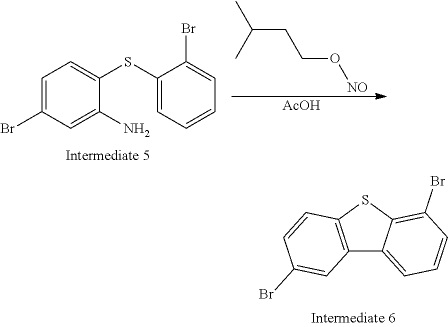

Intermediate 5 (19 g, 52.9 mmol) was dissolved in acetic acid (200 mL), isopentyl nitrite (7 mL, 52.9 mmol) was added, and the system was reacted at room temperature and stirred for 1 h. Then, isopentyl nitrite (7 mL) was supplemented. After the reaction was completed, the system was purified through the column chromatography to obtain Intermediate 6 (2 g, 5.8 mmol, 11%).

Step 3:

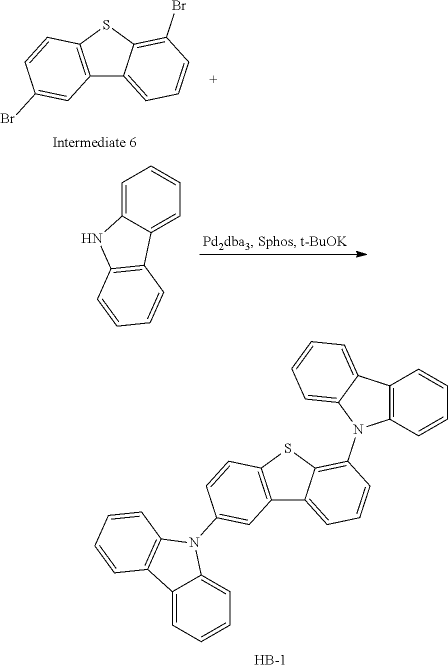

Under nitrogen protection, Intermediate 6 (2 g, 5.8 mmol), carbazole (2.14 g, 12.8 mmol), Pd₂dba₃ (1.06 g, 1.16 mmol), Sphos (950 mg, 2.32 mmol) and t-BuOK (28.8 g, 300 mmol) were mixed, and xylene (50 mL) was added, heated to 140° C. and reacted overnight.

After the reaction was completed, the system was purified through the column chromatography to obtain Compound HB-1 (0.65 g, 1.2 mmol, 21%). The product was identified as the target product with a molecular weight of 514.1.

Those skilled in the art will appreciate that the above preparation methods are merely exemplary. Those skilled in the art can obtain other compound structures of the present disclosure through the modifications of the preparation methods.

To verify the effect of specific molecular structures on energy and electron distribution of compounds, the electrochemical properties of the compounds were measured through cyclic voltammetry. The test was conducted using an electrochemical workstation modelled CorrTest CS120 produced by WUHAN CORRTEST INSTRUMENTS CORP., LTD and using a three-electrode working system where a platinum disk electrode served as a working electrode, a Ag/AgNO₃ electrode served as a reference electrode, and a platinum wire electrode served as an auxiliary electrode. Anhydrous DMF was used as a solvent, 0.1 mol/L tetrabutylammonium hexafluorophosphate was used as a supporting electrolyte, compounds to be tested were prepared into solutions of 10⁻³ mol/L, and nitrogen was introduced into the solution for 10 min for oxygen removal before the test. The parameters of the instrument were set as follows: a scan 5 rate of 100 mV/s, a potential interval of 0.5 mV and a test window of −1 V to −2.9 V.

HOMO and LUMO energy levels of the following compounds were measured through the cyclic voltammetry (CV). The specific results are shown in Table 1.

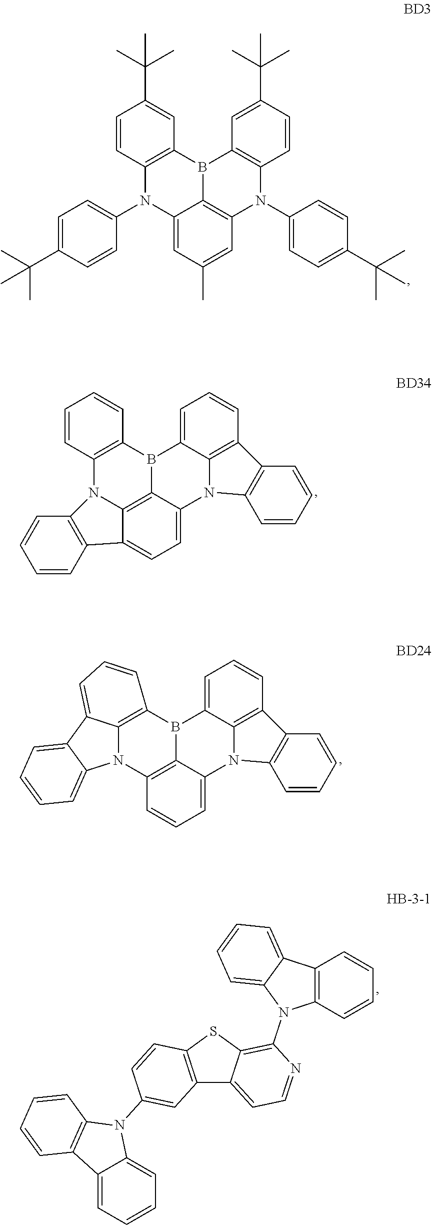 [SMILES: Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1 ; c1ccc2c(c1)B1c3cccc4c5ccccc5n(c34)-c3ccc4c5ccccc5n-2c4c31 ; c1cc2c3c(c1)-n1c4ccccc4c4cccc(c41)B3c1cccc3c4ccccc4n-2c13 ; c1ccc2c(c1)c1ccccc1n2-c1ccc2sc3c(-n4c5ccccc5c5ccccc54)nccc3c2c1]

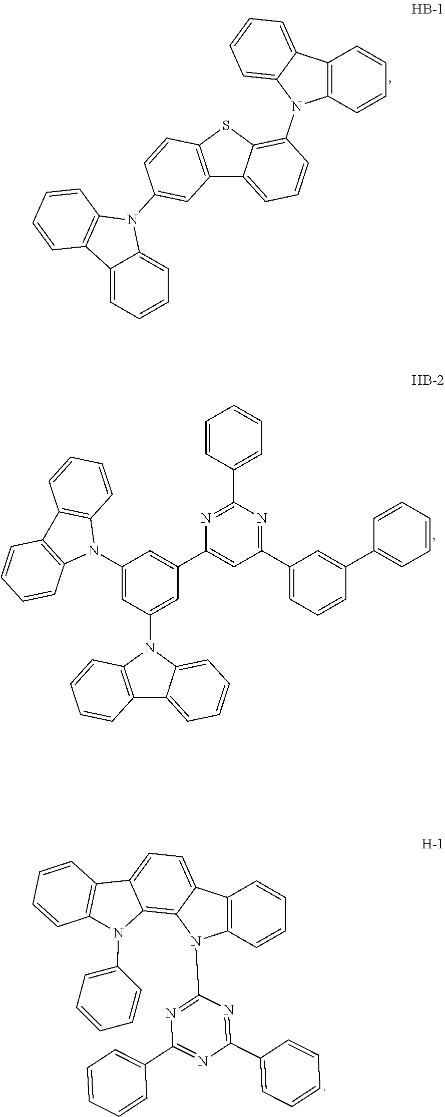 [SMILES: c1cc(-n2c3ccccc3c3ccccc32)c2sc3ccc(-n4c5ccccc5c5ccccc54)cc3c2c1 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1 ; c1ccc(-c2nc(-c3ccccc3)nc(-n3c4ccccc4c4ccc5c6ccccc6n(-c6ccccc6)c5c43)n2)cc1]

TABLE 1
Electrochemical properties of compounds

| Compound | HOMO | LUMO | Compound | HOMO | LUMO |
|---|---|---|---|---|---|
| No. | (eV) | (eV) | No. | (eV) | (eV) |
| BD3 | −5.06 | — | HB-1 | −5.69 | −2.12 |
| BD34 | −5.45 | −2.68 | HB-2 | −5.76 | −2.69 |
| BD24 | −5.46 | −2.71 | H-1 | −5.64 | −2.71 |
| HB-3-1 | −5.71 | −2.47 | | | |

Measurement of Triplet Energy Level

The triplet energy level (T₁) was measured at an ultra-low temperature using characteristics of long-lived triplet excitons. Specifically, a compound was dissolved in a 2-methyltetrahydrofuran solvent to prepare a solution having a concentration of 10⁻⁵ M. The solution was loaded into a quartz sample tube, placed in a Dewar flask and cooled to 77 K. A phosphorescence measurement sample was irradiated with a light source of 350 nm to measure a phosphorescence spectrum. The measurement of the spectrum used a spectrophotometer modelled F98 produced by SHANGHAI LENGGUANG TECH. CO., LTD.

In the phosphorescence spectrum, the longitudinal axis represented a phosphorescence intensity, and the horizontal axis represented a wavelength. A minimum value λ₁ (nm) of a peak on a short wavelength side of the phosphorescence spectrum was taken, and this wavelength value was substituted into the following conversion formula F₁ to calculate the triplet energy.

Tᵢ(eV)=1240/λ₁ Conversion formula F₁:

Triplet energy levels T₁ (eV) of the following compounds were measured through the above method. The specific results are shown in Table 2.

TABLE 2
Triplet energy levels of compounds

| Compound | | Compound | |
|---|---|---|---|
| No. | T₁ (eV) | No. | T₁ (eV) |
| BD3 | 2.67 | HB-1 | 2.81 |
| BD34 | 2.62 | HB-2 | 2.79 |
| BD24 | 2.57 | H-1 | 2.69 |
| HB-3-1 | 2.89 | | |

The electrochemical properties and triplet data of the above compounds are shown in Tables 1 and 2, respectively. Table 1 shows that Compound HB-3-1 has a relatively low LUMO value than Compound HB-1 (−2.47 eV vs −2.12 eV), thereby having a better electron transporting capability. Table 2 shows that the triplet of Compound HB-3-1 is 2.89 eV, which is not only higher than the TADF material used (2.89 eV vs 2.67 eV, 2.62 eV and 2.57 eV), but also higher than the triplets of Compound HB-1, Compound HB-2 and Compound H-1 (2.89 eV vs 2.81 eV, 2.79 eV and 2.69 eV), thereby reducing a loss of triplet excitons, on the basis of exhibiting a hole blocking effect, and thereby improving performance of the device.

The method for preparing an electroluminescent device is not limited. The preparation methods in the following examples are merely examples and not to be construed as limitations. Those skilled in the art can make reasonable improvements on the preparation methods in the following examples based on the related art. Exemplarily, the proportions of various materials in an emissive layer are not particularly limited. Those skilled in the art can reasonably select the proportions within a certain range based on the related art. For example, taking the total weight of the materials in the emissive layer as reference, a host material may account for 80% to 99% and a light-emitting material may account for 1% to 20%; or the host material may account for 90% to 99% and the light-emitting material may account for 1% to 10%; or the host material may account for 95% to 99% and the light-emitting material may account for 1% to 5%. Further, the host material may include one material or two materials, where a ratio of two host materials may be 100:0 to 1:99; or the ratio may be 80:20 to 20:80; or the ratio may be 60:40 to 40:60. In the examples of the device, the characteristics of the device were also tested using conventional equipment in the art (including, but not limited to, evaporator produced by ANGSTROM ENGINEERING, optical testing system produced by SUZHOU FSTAR, life testing system produced by SUZHOU FSTAR, and ellipsometer produced by BEIJING ELLITOP, etc.) by methods well-known to the persons skilled in the art.

### EXAMPLES OF THE DEVICES DESCRIBED IN THE PRESENT APPLICATION

### Device Example 1-1: Preparation of a Blue Delayed Fluorescent Device

Firstly, a glass substrate having an indium tin oxide (ITO) anode with a thickness of 80 nm was cleaned and then treated with oxygen plasma and UV ozone. After the treatment, the substrate was dried in a glovebox to remove moisture. Then, the substrate was mounted on a substrate holder and placed in a vacuum chamber. Organic layers specified below were sequentially deposited through vacuum thermal evaporation on the ITO anode at a rate of 0.2 to 2 Angstroms per second and at a vacuum degree of about 10⁻⁸ torr. Compound HI was used as a hole injection layer (HIL). Compound HT was used as a hole transporting layer (HTL). Compound P-2 was used as an electron blocking layer (EBL). Compound BD3 was doped in Compound P-2 and Compound HB-3-1, all of which were co-deposited for use as an emissive layer (EML). Compound HB-3-1 was used as a hole blocking layer (HBL). On the hole blocking layer, Compound ET and 8-hydroxyquinolinolato-lithium (Liq) were co-deposited for use as an electron transporting layer (ETL). Finally, 8-hydroxyquinolinolato-lithium (Liq) was deposited as an electron injection layer with a thickness of 1 nm and Al was deposited as a cathode with a thickness of 120 nm. The device was transferred back to the glovebox and encapsulated with a glass lid and a moisture getter to complete the device.

### Device Comparative Example 1-1

The implementation mode in Device Comparative Example 1-1 was the same as that in Device Example 1-1, except that in the hole blocking layer (HBL), Compound HB-3-1 was replaced with Compound HB-2.

### Device Comparative Example 1-2

The implementation mode in Device Comparative Example 1-2 was the same as that in Device Example 1-1, except that in the hole blocking layer (HBL), Compound HB-3-1 was replaced with Compound HB-1.

### Device Comparative Example 1-3

The implementation mode in Device Comparative Example 1-3 was the same as that in Device Example 1-1, except that in the emissive layer (EL), Compound P-2 and Compound HB-3-1 were replaced with Compound HB-3-1.

Detailed structures and thicknesses of part of layers of the devices are shown in Table 3. A layer using more than one material is obtained by doping different compounds at their weight ratio as recorded.

TABLE 3
Device structures in device example and device comparative examples

| Device ID | HIL | HTL | EBL | EML | HBL | ETL |
|---|---|---|---|---|---|---|
| Example 1-1 | Compound | Compound | Compound | Compound | Compound | Compound |
| | HI (100 Å) | HT (300 Å) | P-2 (50 Å) | P-2:Compound | HB-3-1 (50 | ET:Liq |
| | | | | HB-3-1:Compound | Å) | (40:60) |
| | | | | BD3 (59%:39%:2%) | | (300 Å) |
| | | | | (300 Å) | | |
| Comparative | Compound | Compound | Compound | Compound | Compound | Compound |
| Example 1-1 | HI (100 Å) | HT (300 Å) | P-2 (50 Å) | P-2:Compound | HB-2 (50 | ET:Liq |
| | | | | HB-3-1:Compound | Å) | (40:60) |
| | | | | BD3 (59%:39%:2%) | | (300 Å) |
| | | | | (300 Å) | | |
| Comparative | Compound | Compound | Compound | Compound | Compound | Compound |
| Example 1-2 | HI (100 Å) | HT (300 Å) | P-2 (50 Å) | P-2:Compound | HB-1 (50 | ET:Liq |
| | | | | HB-3-1:Compound | Å) | (40:60) |
| | | | | BD3 (59%:39%:2%) | | (300 Å) |
| | | | | (300 Å) | | |
| Comparative | Compound | Compound | Compound | Compound | Compound | Compound |
| Example 1-3 | HI (100 Å) | HT (300 Å) | P-2 (50 Å) | HB-3-1:Compound | HB-3-1 (50 | ET:Liq |
| | | | | BD3 (98%:2%) (300 | Å) | (40:60) |
| | | | | Å) | | (300 Å) |

The materials used in the devices have the following structures:

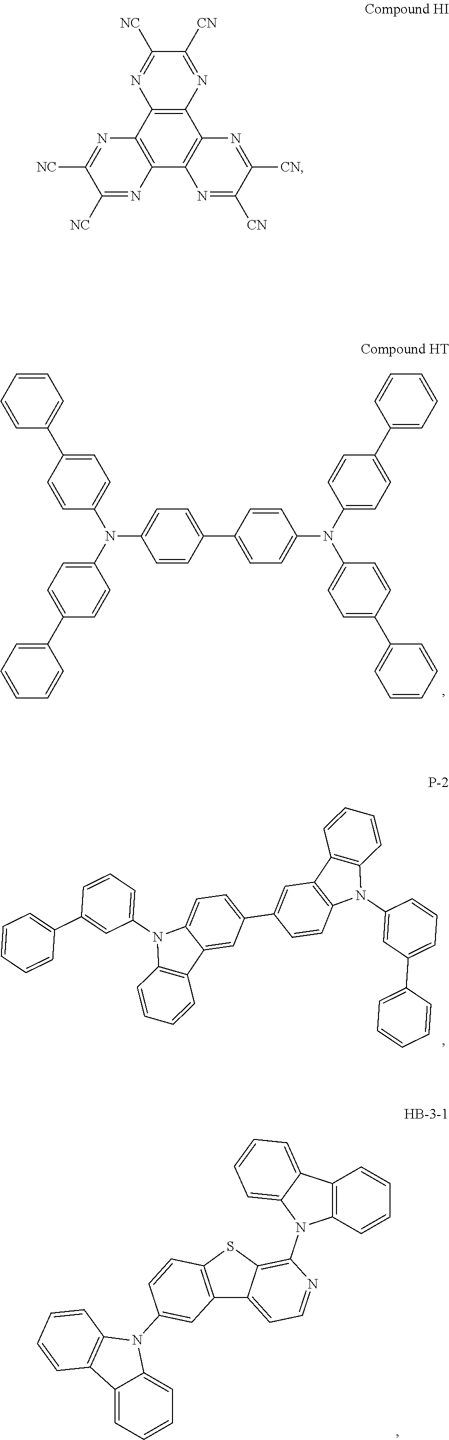 [SMILES: N#Cc1nc2c3nc(C#N)c(C#N)nc3c3nc(C#N)c(C#N)nc3c2nc1C#N ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1 ; c1ccc2c(c1)c1ccccc1n2-c1ccc2sc3c(-n4c5ccccc5c5ccccc54)nccc3c2c1]

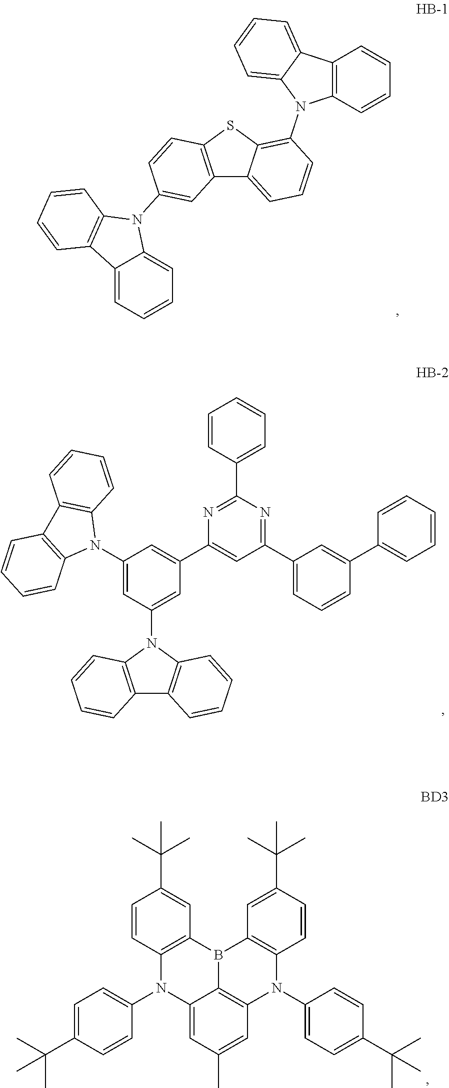 [SMILES: c1cc(-n2c3ccccc3c3ccccc32)c2sc3ccc(-n4c5ccccc5c5ccccc54)cc3c2c1 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1 ; Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1]

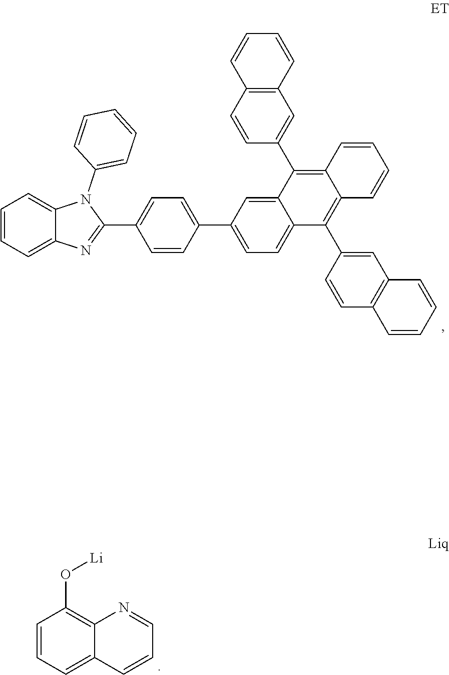

Table 4 shows the measured external quantum efficiency (EQE), maximum emission wavelength (λₘₐₓ), full width at half maximum (FWHM), CIE, CE and lifetime (LT97) data under constant brightness of 10 cd/m².

TABLE 4
Device data

| | | | | | EQE | |
|---|---|---|---|---|---|---|
| Device No. | CIE (x, y) | λₘₐₓ (nm) | FWHM (nm) | CE | (%) | LT97 (h) |
| Example 1-1 | (0.127, 0.106) | 464 | 27.9 | 14.92 | 16.84 | 740.63 |
| Comparative | (0.133, 0.137) | 464 | 29.3 | 9.20 | 8.31 | 698.46 |
| Example 1-1 | | | | | | |
| Comparative | (0.130, 0.110) | 464 | 27.9 | 6.93 | 7.58 | 775.00 |
| Example 1-2 | | | | | | |
| Comparative | (0.126, 0.121) | 466 | 29.5 | 6.27 | 6.51 | 134.00 |
| Example 1-3 | | | | | | |

Discussion

As can be seen from Table 4, the maximum emission wavelengths of the example and the comparative examples are all around 464 nm, and the device of the example and the devices of the comparative examples are all dark blue TADF devices.

In Example 1-1, Compound HB-3-1 of the present disclosure is used as the HBL material. Compared with Comparative Example 1-1 where Compound HB-2 is used as the HBL material, Example 1-1 has a relatively narrow full width at half maximum with significant improvements in current efficiency, EQE and lifetime, and in particular, the EQE of Example 1-1 is improved by more than two times. Compared with Comparative Example 1-2 where Compound HB-1 without a nitrogen-containing heterocyclic ring is used as the HBL material, although the lifetime of Example 1-1 is substantially the same as that of Comparative Example 1-2, the current efficiency and EQE of Example 1-1 are improved by more than two times, respectively, achieving significant improvements. These data sufficiently show the advantages of the HBL material of the present disclosure in the dark blue TADF devices.

In the emissive layer of Example 1-1, Compound P-2 and Compound HB-3-1 are used as dual hosts. Compared with Comparative Example 1-3 where Compound HB-3-1 is used as a single host in the emissive layer, Example 1-1 has a relatively narrow full width at half maximum with significant improvements in current efficiency, EQE and lifetime, where both the current efficiency and the EQE are improved by more than two times, and the lifetime is prolonged by more than five times, sufficiently showing the advantages of the dual hosts over the single host in the TADF devices when the HBL material of the present disclosure is used.

### Device Example 1-2

The implementation mode in Device Example 1-2 was the same as that in Device Example 1-1, except that in the emissive layer (EML), Compound BD3 was replaced with Compound BD34.

### Device Example 1-3

The implementation mode in Device Example 1-3 was the same as that in Device Example 1-2, except that in the emissive layer (EML), Compound HB-3-1 was replaced with Compound N-7.

### Device Comparative Example 1-4

The implementation mode in Device Comparative Example 1-4 was the same as that in Device Example 1-2, except that in the hole blocking layer (HBL), Compound HB-3-1 was replaced with Compound HB-2.

### Device Comparative Example 1-5

The implementation mode in Device Comparative Example 1-5 was the same as that in Device Example 1-2, except that in the hole blocking layer (HBL), Compound HB-3-1 was replaced with Compound HB-1.

### Device Comparative Example 1-6

The implementation mode in Device Comparative Example 1-6 was the same as that in Device Example 1-3, except that in the hole blocking layer (HBL), Compound HB-3-1 was replaced with Compound HB-1.

### Device Comparative Example 1-7

The implementation mode in Device Comparative Example 1-7 was the same as that in Device Example 1-2, except that in the emissive layer (EML), Compound P-2 and Compound HB-3-1 were replaced with Compound HB-3-1.

### Device Comparative Example 1-8

The implementation mode in Device Comparative Example 1-8 was the same as that in Device Example 1-2, except that in the emissive layer (EML), Compound P-2 and Compound HB-3-1 were replaced with Compound P-2.

### Device Comparative Example 1-9

The implementation mode in Device Comparative Example 1-9 was the same as that in Device Example 1-3, except that in the emissive layer (EML), Compound P-2 and Compound N-7 were replaced with Compound N-7.

Detailed structures and thicknesses of part of layers of the devices are shown in Table 5. A layer using more than one material is obtained by doping different compounds at their weight ratio as recorded.

TABLE 5
Device structures in device examples and device comparative examples

| Device ID | HIL | HTL | EBL | EML | HBL | ETL |
|---|---|---|---|---|---|---|
| Example 1-2 | Compound | Compound | Compound | Compound | Compound | Compound |
| | HI (100 Å) | HT (300 Å) | P-2 (50 Å) | P-2:Compound | HB-3-1 (50 | ET:Liq |
| | | | | HB-3-1:Compound BD34 | Å) | (40:60) |
| | | | | (59%:39%:2%) (300 Å) | | (300 Å) |
| Example 1-3 | Compound | Compound | Compound | Compound | Compound | Compound |
| | HI (100 Å) | HT (300 Å) | P-2 (50 Å) | P-2:Compound | HB-3-1 (50 | ET:Liq |
| | | | | N-7:Compound BD34 | Å) | (40:60) |
| | | | | (59%:39%:2%) (300 Å) | | (300 Å) |
| Comparative | Compound | Compound | Compound | Compound | Compound | Compound |
| Example 1-4 | HI (100 Å) | HT (300 Å) | P-2 (50 Å) | P-2:Compound | HB-2 (50 | ET:Liq |
| | | | | HB-3-1:Compound BD34 | Å) | (40:60) |
| | | | | (59%:39%:2%) (300 Å) | | (300 Å) |
| Comparative | Compound | Compound | Compound | Compound | Compound | Compound |
| Example 1-5 | HI (100 Å) | HT (300 Å) | P-2 (50 Å) | P-2:Compound | HB-1 (50 | ET:Liq |
| | | | | HB-3-1:Compound BD34 | Å) | (40:60) |
| | | | | (59%:39%:2%) (300 Å) | | (300 Å) |
| Comparative | Compound | Compound | Compound | Compound | Compound | Compound |
| Example 1-6 | HI (100 Å) | HT (300 Å) | P-2 (50 Å) | P-2:Compound | HB-1 (50 | ET:Liq |
| | | | | N-7:Compound BD34 | Å) | (40:60) |
| | | | | (59%:39%:2%) (300 Å) | | (300 Å) |
| Comparative | Compound | Compound | Compound | Compound | Compound | Compound |
| Example 1-7 | HI (100 Å) | HT (300 Å) | P-2 (50 Å) | HB-3-1:Compound BD34 | HB-3-1 (50 | ET:Liq |
| | | | | (98%:2%) (300 Å) | Å) | (40:60) |
| | | | | | | (300 Å) |
| Comparative | Compound | Compound | Compound | Compound | Compound | Compound |
| Example 1-8 | HI (100 Å) | HT (300 Å) | P-2 (50 Å) | P-2:Compound BD34 | HB-3-1 (50 | ET:Liq |
| | | | | (98%:2%) (300 Å) | Å) | (40:60) |
| | | | | | | (300 Å) |
| Comparative | Compound | Compound | Compound | Compound | Compound | Compound |
| Example 1-9 | HI (100 Å) | HT (300 Å) | P-2 (50 Å) | N-7:Compound BD34 | HB-3-1 (50 | ET:Liq |
| | | | | (98%:2%) (300 Å) | Å) | (40:60) |
| | | | | | | (300 Å) |

The new materials used in the devices have the following structures:

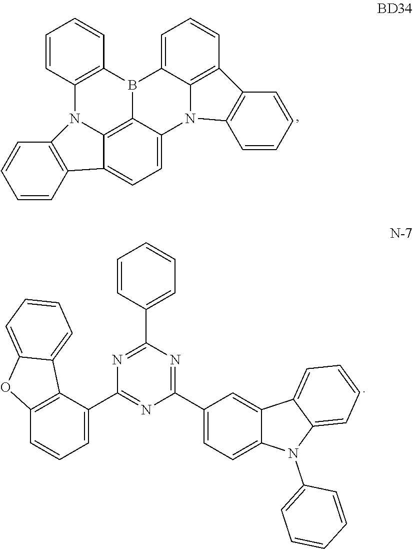 [SMILES: c1ccc2c(c1)B1c3cccc4c5ccccc5n(c34)-c3ccc4c5ccccc5n-2c4c31 ; c1ccc(-c2nc(-c3ccc4c(c3)c3ccccc3n4-c3ccccc3)nc(-c3cccc4oc5ccccc5c34)n2)cc1]

Table 6 shows the measured external quantum efficiency (EQE), maximum emission wavelength (λₘₐₓ), full width at half maximum (FWHM), CIE, CE and lifetime (LT97) data under constant brightness of 10 cd/in².

TABLE 6
Device data

| | | | | | | LT97 |
|---|---|---|---|---|---|---|
| Device No. | CIE (x, y) | λₘₐₓ (nm) | FWHM (nm) | CE | EQE (%) | (h) |
| Example 1-2 | (0.123, 0.269) | 477 | 44.1 | 11.04 | 6.71 | 1293.09 |
| Example 1-3 | (0.142, 0.304) | 477 | 46.2 | 20.73 | 11.32 | 6203.00 |
| Comparative | (0.124, 0.280) | 476 | 43.6 | 8.11 | 5.00 | 797.24 |
| Example 1-4 | | | | | | |
| Comparative | (0.164, 0.298) | 475 | 49.1 | 2.23 | 1.20 | 269.00 |
| Example 1-5 | | | | | | |
| Comparative | (0.141, 0.300) | 476 | 45.6 | 17.22 | 9.49 | 1088.00 |
| Example 1-6 | | | | | | |
| Comparative | (0.125, 0.276) | 477 | 44.1 | 8.67 | 5.16 | 710.00 |
| Example 1-7 | | | | | | |
| Comparative | (0.141, 0.311) | 476 | 48.2 | 9.80 | 5.22 | 987.00 |
| Example 1-8 | | | | | | |
| Comparative | (0.134, 0.271) | 475 | 41.1 | 8.71 | 5.18 | 928.00 |
| Example 1-9 | | | | | | |

Discussion

As can be seen from Table 6, the maximum emission wavelengths of the examples and the comparative examples are all around 477 nm, and the devices of the examples and the devices of the comparative examples are all sky blue TADF devices.

In the emissive layer of Example 1-2, Compound P-2 and Compound HB-3-1 are used as dual hosts, and Compound HB-3-1 of the present disclosure is used as the HBL material. Compared with Comparative Example 1-4 where the same dual host materials are used in the emissive layer and Compound HB-2 is used as the HBL material, the current efficiency, EQE and lifetime of Example 1-2 are all improved significantly, where the current efficiency is improved by 36.1%, the EQE is improved by 34.2%, and the lifetime is prolonged by 62.2%. Compared with Comparative Example 1-5 where the same dual host materials are used in the emissive layer and Compound HB-1 without a nitrogen-containing heterocyclic ring is used as the HBL material, the current efficiency, EQE and lifetime of Example 1-2 are all significantly improved, where the current efficiency is improved by nearly five times, the EQE is improved by more than five times, and the lifetime is prolonged by more than four times. In the emissive layer of Example 1-3, Compound P-2 and Compound N-7 are used as dual hosts, and Compound HB-3-1 of the present disclosure is used as the HBL material. Compared with Comparative Example 1-6 where the same dual host materials are used in the emissive layer and Compound HB-1 without a nitrogen-containing heterocyclic ring is used as the HBL material, the current efficiency of Example 1-3 is improved by 20.4%, the EQE of Example 1-3 is improved by 19.3%, and in particular, the lifetime of Example 1-3 is significantly prolonged by nearly six times. These data sufficiently show the advantages of the HBL material of the present disclosure in the sky blue TADF devices.

In the emissive layer of Example 1-2, Compound P-2 and Compound HB-3-1 are used as the dual hosts. Compared with Comparative Example 1-7 where Compound HB-3-1 is used as a single host in the emissive layer and Comparative Example 1-8 where Compound P-2 is used as a single host in the emissive layer, the current efficiency, EQE and lifetime of Example 1-2 are all significantly improved with a maximum improvement in current efficiency of 27.3%, a maximum improvement in EQE of 30% and a maximum prolongation in lifetime of 82.1%. In the emissive layer of Example 1-3, Compound P-2 and Compound N-7 are used as the dual hosts. Compared with Comparative Example 1-8 where Compound P-2 is used as the single host in the emissive layer and Comparative Example 1-9 where Compound N-7 is used as a single host in the emissive layer, the current efficiency, EQE and lifetime of Example 1-4 are all significantly improved with maximum improvements in current efficiency and EQE of more than two times, respectively, and a maximum prolongation in lifetime of more than six times. These data sufficiently show the advantages of the dual hosts over the single hosts in the TADF devices when the HBL material of the present disclosure is used.

### Device Example 1-4

The implementation mode in Device Example 1-4 was the same as that in Device Example 1-1, except that in the emissive layer (EML), Compound BD3 was replaced with Compound BD24.

### Device Example 1-5

The implementation mode in Device Example 1-5 was the same as that in Device Example 1-4, except that in the emissive layer (EML), Compound HB-3-1 was replaced with Compound N-7.

### Device Comparative Example 1-10

The implementation mode in Device Comparative Example 1-10 was the same as that in Device Example 1-4, except that in the hole blocking layer (HBL), Compound HB-3-1 was replaced with Compound HB-2.

### Device Comparative Example 1-11

The implementation mode in Device Comparative Example 1-11 was the same as that in Device Example 1-4, except that in the hole blocking layer (HBL), Compound HB-3-1 was replaced with Compound H-1.

### Device Comparative Example 1-12

The implementation mode in Device Comparative Example 1-12 was the same as that in Device Example 1-4, except that in the hole blocking layer (HBL), Compound HB-3-1 was replaced with Compound HB-1.

### Device Comparative Example 1-13

The implementation mode in Device Comparative Example 1-13 was the same as that in Device Example 1-4, except that in the emissive layer (EML), Compound P-2 and Compound HB-3-1 were replaced with Compound HB-3-1.

Detailed structures and thicknesses of part of layers of the devices are shown in Table 7. A layer using more than one material is obtained by doping different compounds at their weight ratio as recorded.

TABLE 7
Device structures in device examples and device comparative examples

| Device ID | HIL | HTL | EBL | EML | HBL | ETL |
|---|---|---|---|---|---|---|
| Example 1-4 | Compound | Compound | Compound | Compound | Compound | Compound |
| | HI (100 Å) | HT (300 Å) | P-2 (50 Å) | P-2:Compound | HB-3-1 (50 | ET:Liq |
| | | | | HB-3-1:Compound | Å) | (40:60) (300 |
| | | | | BD24 (59%:39%:2%) | | Å) |
| | | | | (300 Å) | | |
| Example 1-5 | Compound | Compound | Compound | Compound | Compound | Compound |
| | HI (100 Å) | HT (300 Å) | P-2 (50 Å) | P-2:Compound | HB-3-1 (50 | ET:Liq |
| | | | | N-7:Compound BD24 | Å) | (40:60) (300 |
| | | | | (59%:39%:2%) (300 Å) | | Å) |
| Comparative | Compound | Compound | Compound | Compound | Compound | Compound |
| Example | HI (100 Å) | HT (300 Å) | P-2 (50 Å) | P-2:Compound | HB-2 (50 | ET:Liq |
| 1-10 | | | | HB-3-1:Compound | Å) | (40:60) (300 |
| | | | | BD24 (59%:39%:2%) | | Å) |
| | | | | (300 Å) | | |
| Comparative | Compound | Compound | Compound | Compound | Compound | Compound |
| Example | HI (100 Å) | HT (300 Å) | P-2 (50 Å) | P-2:Compound | H-1 (50 Å) | ET:Liq |
| 1-11 | | | | HB-3-1:Compound | | (40:60) (300 |
| | | | | BD24 (59%:39%:2%) | | Å) |
| | | | | (300 Å) | | |
| Comparative | Compound | Compound | Compound | Compound | Compound | Compound |
| Example | HI (100 Å) | HT (300 Å) | P-2 (50 Å) | P-2:Compound | HB-1 (50 | ET:Liq |
| 1-12 | | | | HB-3-1:Compound | Å) | (40:60) (300 |
| | | | | BD24 (59%:39%:2%) | | Å) |
| | | | | (300 Å) | | |
| Comparative | Compound | Compound | Compound | Compound | Compound | Compound |
| Example | HI (100 Å) | HT (300 Å) | P-2 (50 Å) | HB-3-1:Compound | HB-3-1 (50 | ET:Liq |
| 1-13 | | | | BD24 (98%:2%) (300 Å) | Å) | (40:60) (300 |
| | | | | | | Å) |

The new materials used in the devices have the following structures:

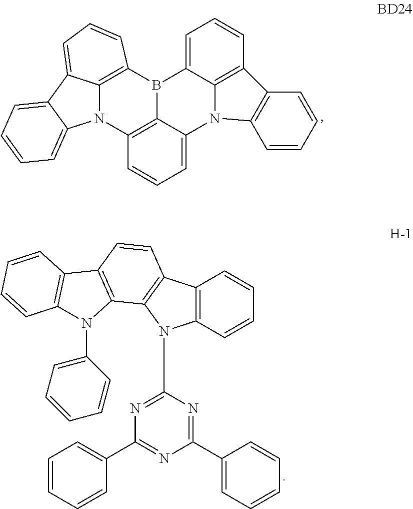 [SMILES: c1cc2c3c(c1)-n1c4ccccc4c4cccc(c41)B3c1cccc3c4ccccc4n-2c13 ; c1ccc(-c2nc(-c3ccccc3)nc(-n3c4ccccc4c4ccc5c6ccccc6n(-c6ccccc6)c5c43)n2)cc1]

Table 8 shows the measured external quantum efficiency (EQE), maximum emission wavelength (λₘₐₓ), full width at half maximum (FWHM), CIE and CE data under constant brightness of 300 cd/in² and the lifetime (LT97) data under constant brightness of 10 cd/in².

TABLE 8
Device data

| | | | | | | LT97 |
|---|---|---|---|---|---|---|
| Device No. | CIE (x, y) | λₘₐₓ (nm) | FWHM (nm) | CE | EQE (%) | (h) |
| Example 1-4 | (0.111, 0.382) | 485 | 35.4 | 21.68 | 11.21 | 9012.43 |
| Example 1-5 | (0.113, 0.369) | 485 | 32.9 | 28.71 | 15.30 | 14931.00 |
| Comparative | (0.114, 0.385) | 485 | 36.2 | 13.75 | 7.02 | 2595.26 |
| Example 1-10 | | | | | | |
| Comparative | (0.141, 0.408) | 486 | 37.9 | 8.06 | 3.84 | 3635.83 |
| Example 1-11 | | | | | | |
| Comparative | (0.148, 0.389) | 486 | 39.7 | 2.92 | 1.41 | 735.00 |
| Example 1-12 | | | | | | |
| Comparative | (0.120, 0.423) | 488 | 38.2 | 17.95 | 8.54 | 3304.00 |
| Example 1-13 | | | | | | |

Discussion

As can be seen from Table 8, the maximum emission wavelengths of the examples and the comparative examples are all around 485 nm, and the devices of the examples and the devices of the comparative examples are all light blue TADF devices.

In Example 1-4, Compound HB-3-1 of the present disclosure is used as the HBL material. Compared with Comparative Example 1-10 where Compound HB-2 is used as the HBL material, the current efficiency and EQE of Example 1-4 are improved by 57.7% and 59.7%, respectively, and the lifetime of Example 1-4 is significantly improved, which is prolonged by more than three times. Compared with Comparative Example 1-11 where Compound H-1 is used as the HBL material, the current efficiency, EQE and lifetime of Example 1-4 are all improved by more than two times. Compared with Comparative Example 1-12 where Compound HB-1 without a nitrogen-containing heterocyclic ring is used as the HBL material, both the current efficiency and the EQE of Example 1-4 are significantly improved by more than seven times, and in particular, the lifetime of Example 1-4 is prolonged by more than twelve times, achieving a significant improvement. In addition, in the emissive layer of Example 1-5, Compound P-2 and Compound N-7 are used as the dual hosts, and Compound HB-3-1 of the present disclosure is used as the HBL material. Compared with Example 1-4, the current efficiency, EQE and lifetime of Example 1-5 are further improved. These data sufficiently show the advantages of the HBL material of the present disclosure in the light blue TADF devices.

In the emissive layer of Example 1-4, Compound P-2 and Compound HB-3-1 are used as the dual hosts. Compared with Comparative Example 1-13 where Compound HB-3-1 is used as a single host in the emissive layer, the current efficiency of Example 1-4 is improved by 20.8%, the EQE of Example 1-4 is improved by 31.2%, and the lifetime of Example 1-4 is prolonged by nearly three times, improving overall performance of the device. These data sufficiently show the advantages of the dual hosts over the single host in the TADF devices when the HBL material of the present disclosure is used.

In conclusion, it can be seen that no matter the dark blue TADF devices, sky blue TADF devices or the light blue TADF devices of the present disclosure, the devices using the specific compounds of the present disclosure as an HBL material are improved in current efficiency, EQE and lifetime and can obtain excellent overall device performance.

### Device Example 2-1: Preparation of a Green Phosphorescent Device

Firstly, a glass substrate having an indium tin oxide (ITO) anode with a thickness of 80 nm was cleaned and then treated with oxygen plasma and UV ozone. After the treatment, the substrate was dried in a glovebox to remove moisture. Then, the substrate was mounted on a substrate holder and placed in a vacuum chamber. Organic layers specified below were sequentially deposited through vacuum thermal evaporation on the ITO anode at a rate of 0.2 to 2 Angstroms per second and at a vacuum degree of about 10⁻⁸ torr. Compound HI was used as a hole injection layer (HIL). Compound HT was used as a hole transporting layer (HTL). Compound X-4 was used as an electron blocking layer (EBL). Compound GD4 was doped in Compound X-4 and Compound H-12, all of which were co-deposited for use as an emissive layer (EML). Compound HB-3-1 was used as a hole blocking layer (HBL). On the hole blocking layer, Compound ET and 8-hydroxyquinolinolato-lithium (Liq) were co-deposited for use as an electron transporting layer (ETL). Finally, 8-hydroxyquinolinolato-lithium (Liq) was deposited as an electron injection layer with a thickness of 1 nm and Al was deposited as a cathode with a thickness of 120 nm. The device was transferred back to the glovebox and encapsulated with a glass lid and a moisture getter to complete the device.

### Device Comparative Example 2-1

The implementation mode in Device Comparative Example 2-1 was the same as that in Device Example 2-1, except that in the hole blocking layer (HBL), Compound HB-3-1 was replaced with Compound HB-2.

### Device Comparative Example 2-2

The implementation mode in Device Comparative Example 2-2 was the same as that in Device Example 2-1, except that in the hole blocking layer (HBL), Compound HB-3-1 was replaced with Compound H-1.

### Device Comparative Example 2-3

The implementation mode in Device Comparative Example 2-3 was the same as that in Device Example 2-1, except that in the hole blocking layer (HBL), Compound HB-3-1 was replaced with Compound HB-1.

Detailed structures and thicknesses of part of layers of the devices are shown in Table 9. A layer using more than one material is obtained by doping different compounds at their weight ratio as recorded.

TABLE 9
Device structures in device example and device comparative examples

| Device ID | HIL | HTL | EBL | EML | HBL | ETL |
|---|---|---|---|---|---|---|
| Example 2-1 | Compound | Compound | Compound | Compound | Compound | Compound |
| | HI (100 Å) | HT (350 Å) | X-4 (50 Å) | X-4:Compound | HB-3-1 (50 | ET:Liq |
| | | | | H-12:Compound | Å) | (40:60) (300 |
| | | | | GD4 (46%:46%:8%) | | Å) |
| | | | | (400 Å) | | |
| Comparative | Compound | Compound | Compound | Compound | Compound | Compound |
| Example 2-1 | HI (100 Å) | HT (350 Å) | X-4 (50 Å) | X-4:Compound | HB-2 (50 Å) | ET:Liq |
| | | | | H-12:Compound | | (40:60) (300 |
| | | | | GD4 (46%:46%:8%) | | Å) |
| | | | | (400 Å) | | |
| Comparative | Compound | Compound | Compound | Compound | Compound | Compound |
| Example 2-2 | HI (100 Å) | HT (350 Å) | X-4 (50 Å) | X-4:Compound | H-1 (50 Å) | ET:Liq |
| | | | | H-12:Compound | | (40:60) (300 |
| | | | | GD4 (46%:46%:8%) | | Å) |
| | | | | (400 Å) | | |
| Comparative | Compound | Compound | Compound | Compound | Compound | Compound |
| Example 2-3 | HI (100 Å) | HT (350 Å) | X-4 (50 Å) | X-4:Compound | HB-1 (50 Å) | ET:Liq |
| | | | | H-12:Compound | | (40:60) (300 |
| | | | | GD4 (46%:46%:8%) | | Å) |
| | | | | (400 Å) | | |

The new materials used in the devices have the following structures:

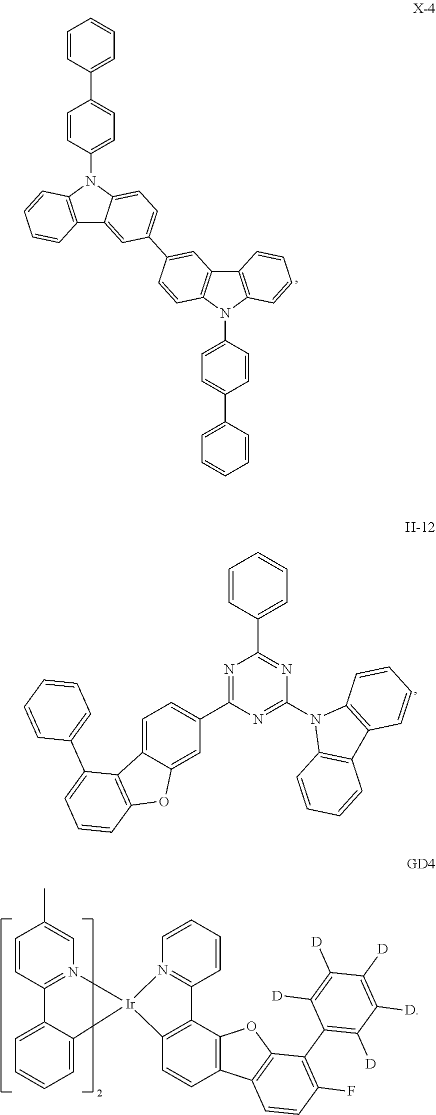

Table 10 shows that the measured current efficiency (CE), external quantum efficiency (EQE), maximum emission wavelength (λₘₐₓ), full width at half maximum (FWHM) and CIE data under constant brightness of 1000 cd/in² and the lifetime (LT97) data under a constant current of 80 mA/cm².

TABLE 10
Device data

| | | λₘₐₓ | FWHM | | EQE | LT97 |
|---|---|---|---|---|---|---|
| Device No. | CIE (x, y) | (nm) | (nm) | CE | (%) | (h) |
| Example 2-1 | (0.362, 0.615) | 531 | 62.1 | 84.86 | 22.34 | 39.20 |
| Comparative | (0.362, 0.615) | 531 | 62.4 | 80.58 | 21.27 | 28.86 |
| Example 2-1 | | | | | | |
| Comparative | (0.361, 0.616) | 531 | 61.6 | 85.00 | 22.33 | 41.00 |
| Example 2-2 | | | | | | |
| Comparative | (0.354, 0.621) | 530 | 59.8 | 87.39 | 22.83 | 20.20 |
| Example 2-3 | | | | | | |

Discussion

In Example 2-1, Compound HB-3-1 of the present disclosure is used as the HBL material, and Compound HB-2 and Compound H-1, which are commercially available HBL materials in green phosphorescent devices, are used in Comparative Example 2-1 and Comparative Example 2-2. The current efficiency, EQE and lifetime of the example are substantially the same as those of the comparative examples, indicating that using the compound of the present disclosure as the HBL material in the green phosphorescent devices, as the commercially available HBL materials in the green phosphorescent devices, can also obtain relatively good device performance. In Comparative Example 2-3, Compound HB-1 without a nitrogen-containing heterocyclic ring is used as the HBL material. The current efficiency and EQE of Example 2-1 are substantially the same as those of Comparative Example 2-3, but the lifetime of Example 2-1 is significantly improved compared with that of Comparative Example 2-3, which is prolonged by 94%. These data prove that using the HBL material of the present disclosure can also obtain excellent device performance in the green phosphorescent devices.

### Device Example 3-1: Preparation of a Red Phosphorescent Device

Firstly, a glass substrate having an indium tin oxide (ITO) anode with a thickness of 80 nm was cleaned and then treated with oxygen plasma and UV ozone. After the treatment, the substrate was dried in a glovebox to remove moisture. Then, the substrate was mounted on a substrate holder and placed in a vacuum chamber. Organic layers specified below were sequentially deposited through vacuum thermal evaporation on the ITO anode at a rate of 0.2 to 2 Angstroms per second and at a vacuum degree of about 10⁻⁸ torr. Compound HI was used as a hole injection layer (HIL). Compound HT was used as a hole transporting layer (HTL). Compound EB was used as an electron blocking layer (EBL). Compound RD28 was doped in Compound RH, which were co-deposited for use as an emissive layer (EML). Compound HB-3-1 was used as a hole blocking layer (HBL). On the hole blocking layer, Compound ET and 8-hydroxyquinolinolato-lithium (Liq) were co-deposited for use as an electron transporting layer (ETL). Finally, 8-hydroxyquinolinolato-lithium (Liq) was deposited as an electron injection layer with a thickness of 1 nm and Al was deposited as a cathode with a thickness of 120 nm. The device was transferred back to the glovebox and encapsulated with a glass lid and a moisture getter to complete the device.

### Device Comparative Example 3-1

The implementation mode in Device Comparative Example 3-1 was the same as that in Device Example 3-1, except that in the hole blocking layer (HBL), Compound HB-3-1 was replaced with Compound HB-2.

### Device Comparative Example 3-2

The implementation mode in Device Comparative Example 3-2 was the same as that in Device Example 3-1, except that in the hole blocking layer (HBL), Compound HB-3-1 was replaced with Compound H-1.

### Device Comparative Example 3-3

The implementation mode in Device Comparative Example 3-3 was the same as that in Device Example 3-1, except that in the hole blocking layer (HBL), Compound HB-3-1 was replaced with Compound HB-1.

Detailed structures and thicknesses of part of layers of the devices are shown in Table 11. A layer using more than one material is obtained by doping different compounds at their weight ratio as recorded.

TABLE 11
Device structures in device example and device comparative examples

| Device ID | HIL | HTL | EBL | EML | HBL | ETL |
|---|---|---|---|---|---|---|
| Example 3-1 | Compound | Compound | Compound | Compound | Compound | Compound |
| | HI (100 Å) | HT (400 Å) | EB (50 Å) | RH:Compound RD28 | HB-3-1 (50 | ET:Liq |
| | | | | (98%:2%) (400 Å) | Å) | (40:60) |
| | | | | | | (300 Å) |
| Comparative | Compound | Compound | Compound | Compound | Compound | Compound |
| Example 3-1 | HI (100 Å) | HT (400 Å) | EB (50 Å) | RH:Compound RD28 | HB-2 (50 | ET:Liq |
| | | | | (98%:2%) (400 Å) | Å) | (40:60) |
| | | | | | | (300 Å) |
| Comparative | Compound | Compound | Compound | Compound | Compound | Compound |
| Example 3-2 | HI (100 Å) | HT (400 Å) | EB (50 Å) | RH:Compound RD28 | H-1 (50 Å) | ET:Liq |
| | | | | (98%:2%) (400 Å) | | (40:60) |
| | | | | | | (300 Å) |
| Comparative | Compound | Compound | Compound | Compound | Compound | Compound |
| Example 3-3 | HI (100 Å) | HT (400 Å) | EB (50 Å) | RH:Compound RD28 | HB-1 (50 | ET:Liq |
| | | | | (98%:2%) (400 Å) | Å) | (40:60) |
| | | | | | | (300 Å) |

The new materials used in the devices have the following structures:

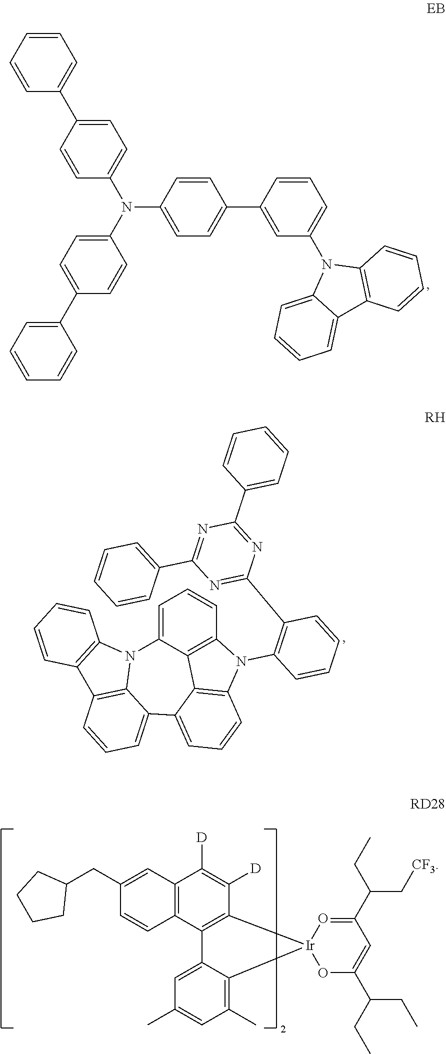

Table 12 shows that the measured external quantum efficiency (EQE), maximum emission wavelength (λₘₐₓ), full width at half maximum (FWHM), CIE and CE data under constant brightness of 1000 cd/m² and the lifetime (LT97) data under a constant current of 80 mA/cm².

TABLE 12
Device data

| | | | | | | LT97 |
|---|---|---|---|---|---|---|
| Device No. | CIE (x, y) | λₘₐₓ (nm) | FWHM (nm) | CE | EQE (%) | (h) |
| Example 3-1 | (0.678, 0.321) | 620 | 49.7 | 25.39 | 26.86 | 110.00 |
| Comparative | (0.678, 0.322) | 621 | 49.9 | 25.27 | 26.92 | 109.00 |
| Example 3-1 | | | | | | |
| Comparative | (0.678, 0.321) | 621 | 49.2 | 25.49 | 26.80 | 109.00 |
| Example 3-2 | | | | | | |
| Comparative | (0.677, 0.322) | 621 | 48.1 | 25.43 | 26.55 | 25.00 |
| Example 3-3 | | | | | | |

Discussion

In Example 3-1, Compound HB-3-1 of the present disclosure is used as the HBL material, and Compound HB-2 and Compound H-1, which are commercially available HBL materials in red phosphorescent devices, are used in Comparative Example 3-1 and Comparative Example 3-2. The current efficiency, EQE and lifetime of the example are substantially the same as those of the comparative examples, indicating that using the compound of the present disclosure as the HBL material in the red phosphorescent devices, as the commercially available HBL materials in the red phosphorescent devices, can also obtain relatively good device performance In Comparative Example 3-3, Compound HB-1 without a nitrogen-containing heterocyclic ring is used as the HBL material. The current efficiency and EQE of Example 3-1 are substantially the same as those of Comparative Example 3-3, but the lifetime of Example 3-1 is significantly improved compared with that of Comparative Example 3-3, which is prolonged by more than 3 times. These data prove that using the specific compound of the present disclosure as the HBL material can obtain excellent device performance in the red phosphorescent devices.

In conclusion, using the specific compounds of the present disclosure as the HBL materials for application to blue TADF devices, green phosphorescent devices and red phosphorescent devices can obtain excellent device effects. Therefore, the HBL material can be used as a general HBL and is suitable for a light-emitting device comprising OLEDs of three colors: red, green and blue. Such a light-emitting device can not only reduce types of materials, but also improve performance of the light-emitting device, such as higher efficiency and longer lifetime.

It is to be understood that various embodiments described herein are merely examples and not intended to limit the scope of the present disclosure. Therefore, it is apparent to those skilled in the art that the present disclosure as claimed may include variations of specific embodiments and preferred embodiments described herein. Many of the materials and structures described herein may be replaced with other materials and structures without departing from the spirit of the present disclosure. It is to be understood that various theories as to why the present disclosure works are not intended to be limiting.

What is claimed is:

1. A blue delayed fluorescent device, comprising:
an anode,
a cathode,
an emissive layer disposed between the anode and the cathode, and
a hole blocking layer disposed between the emissive layer and the cathode, wherein the hole blocking layer comprises a hole blocking material;
wherein the hole blocking material is a compound having a structure represented by Formula 1: 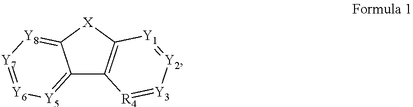
wherein in Formula 1,
X is selected from O, S, Se, SiRR or PR;
R is, at each occurrence identically or differently, selected from the group consisting of: substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted aryl having 6 to 30 carbon atoms, and substituted or unsubstituted heteroaryl having 3 to 30 carbon atoms; when multiple R are present at the same time, the multiple R are the same or different;
Y₁ to Y₈ are, at each occurrence identically or differently, selected from CR₁, CR₂ or N; at least one of Y₁ to Y₈ is selected from N, and at least one of Y₁ to Y₈ is selected from CR₁;
R₁ has a structure represented by Formula 2: 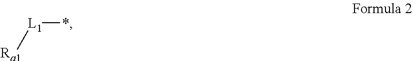
wherein Rₐ₁ is, at each occurrence identically or differently, selected from substituted or unsubstituted aryl having 6 to 30 carbon atoms, substituted or unsubstituted heteroaryl having 3 to 30 carbon atoms or a combination thereof;
L₁ is, at each occurrence identically or differently, selected from a single bond, substituted or unsubstituted arylene having 6 to 30 carbon atoms, substituted or unsubstituted heteroarylene having 3 to 30 carbon atoms or a combination thereof;
* represents a position where the substituent having the structure of Formula 2 is joined to Formula 1;
R₂ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
adjacent substituents R, R₂ can be optionally joined to form a ring; the emissive layer comprises a first host material, a second host material and a blue delayed fluorescent material, wherein the triplet energy level of the first host material and the triplet energy level of the second host material are each greater than that of the blue delayed fluorescent material;
the blue delayed fluorescent material has a structure represented by Formula 3: 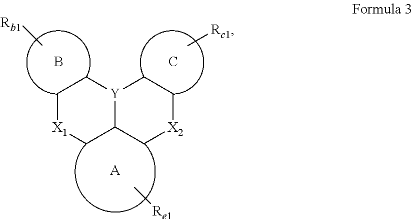
wherein in Formula 3,
the ring A, the ring B and the ring C are each independently selected from an aromatic ring having 6 to 30 carbon atoms, a heteroaromatic ring having 3 to 30 carbon atoms or a combination thereof;
Y is selected from B, P═O, P═S, Al, Ga, As, SiR′ or GeR′;
X₁ and X₂ are each independently selected from O, N—R_{d1}, S or Se, wherein N—R_{d1} has a structure represented by Formula 4: 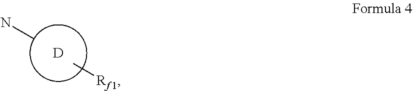
wherein the ring D is, at each occurrence identically or differently, selected from an aromatic ring having 6 to 30 carbon atoms, a heteroaromatic ring having 3 to 30 carbon atoms or a combination thereof;
R_{b1}, R_{c1}, Rₑ₁ and R_{f1} represent, at each occurrence identically or differently, mono-substitution, multiple substitutions or non-substitution;
R′, R_{b1}, R_{c1}, Rₑ₁ and R_{f1} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and
adjacent substituents R_{b1}, R_{c1}, Rₑ₁, R_{f1} can be optionally joined to form a ring.

2. The blue delayed fluorescent device according to claim 1, wherein X is selected from O, S or Se; preferably, X is selected from O or S; more preferably, X is selected from S.

3. The blue delayed fluorescent device according to claim 1, wherein at least two of Y₁ to Y₈ are selected from CR₁; preferably, two and only two of Y₁ to Y₈ are selected from CR₁.

4. The blue delayed fluorescent device according to claim 1, wherein one and only one of Y₁ to Ys is selected from N.

5. The blue delayed fluorescent device according to claim 1, wherein L₁ is, at each occurrence identically or differently, selected from a single bond, substituted or unsubstituted arylene having 6 to 12 carbon atoms, substituted or unsubstituted heteroarylene having 3 to 12 carbon atoms or a combination thereof; preferably, L₁ is, at each occurrence identically or differently, selected from a single bond.

6. The blue delayed fluorescent device according to claim 1, wherein Rₐ₁ is, at each occurrence identically or differently, selected from substituted or unsubstituted aryl having 6 to 24 carbon atoms, substituted or unsubstituted heteroaryl having 3 to 18 carbon atoms or a combination thereof;
preferably, Rₐ₁ is, at each occurrence identically or differently, selected from the group consisting of: dibenzothienyl, dibenzofuryl, dibenzoselenophenyl, furyl, thienyl, benzofuryl, benzothienyl, benzoselenophenyl, carbazolyl, indolocarbazolyl, pyridoindolyl, pyrrolopyridinyl, pyrazolyl, imidazolyl, triazolyl, oxazolyl, thiazolyl, oxadiazolyl, oxatriazolyl, dioxazolyl, thiadiazolyl, pyridyl, pyridazinyl, pyrimidinyl, pyrazinyl, triazinyl, oxazinyl, oxathiazinyl, oxadiazinyl, indolyl, benzimidazolyl, indazolyl, indenoazinyl, benzoxazolyl, benzisoxazolyl, benzothiazolyl, quinolyl, isoquinolyl, cinnolinyl, quinazolinyl, quinoxalinyl, naphthyridinyl, phthalazinyl, pteridinyl, xanthenyl, acridinyl, phenazinyl, phenothiazinyl, benzofuropyridinyl, furodipyridinyl, benzothienopyridinyl, thienodipyridinyl, benzoselenophenopyridinyl and selenophenodipyridinyl; and
more preferably, Rₐ₁ is, at each occurrence identically or differently, selected from the group consisting of: dibenzothienyl, dibenzofuryl, dibenzoselenophenyl, carbazolyl, indolocarbazolyl, imidazolyl, pyridyl, triazinyl or benzimidazolyl.

7. The blue delayed fluorescent device according to claim 1, wherein the hole blocking material is selected from the group consisting of the following:

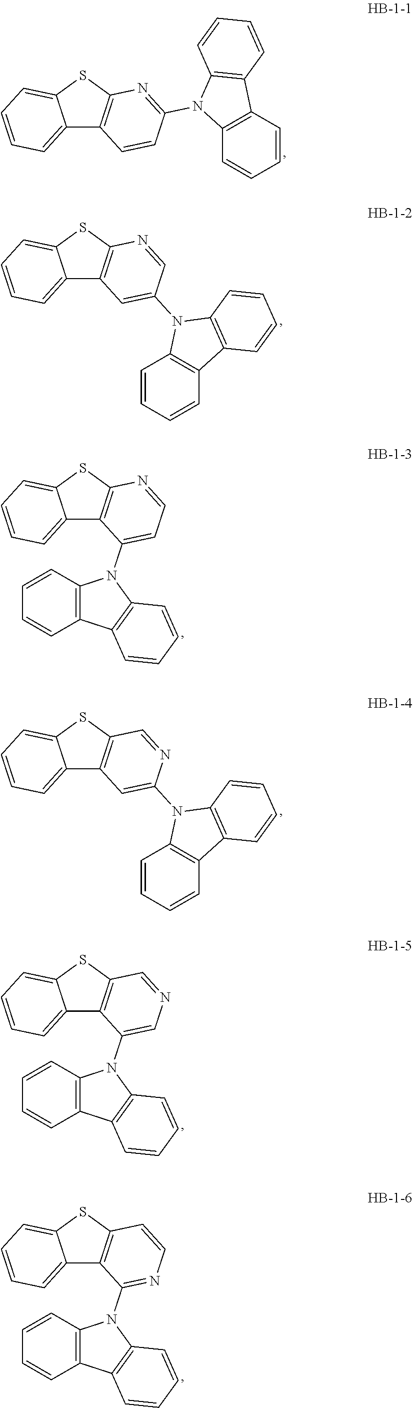 [SMILES: c1ccc2c(c1)sc1nc(-n3c4ccccc4c4ccccc43)ccc12 ; c1ccc2c(c1)sc1ncc(-n3c4ccccc4c4ccccc43)cc12 ; c1ccc2c(c1)sc1nccc(-n3c4ccccc4c4ccccc43)c12 ; c1ccc2c(c1)sc1cnc(-n3c4ccccc4c4ccccc43)cc12 ; c1ccc2c(c1)sc1cncc(-n3c4ccccc4c4ccccc43)c12 ; c1ccc2c(c1)sc1ccnc(-n3c4ccccc4c4ccccc43)c12]

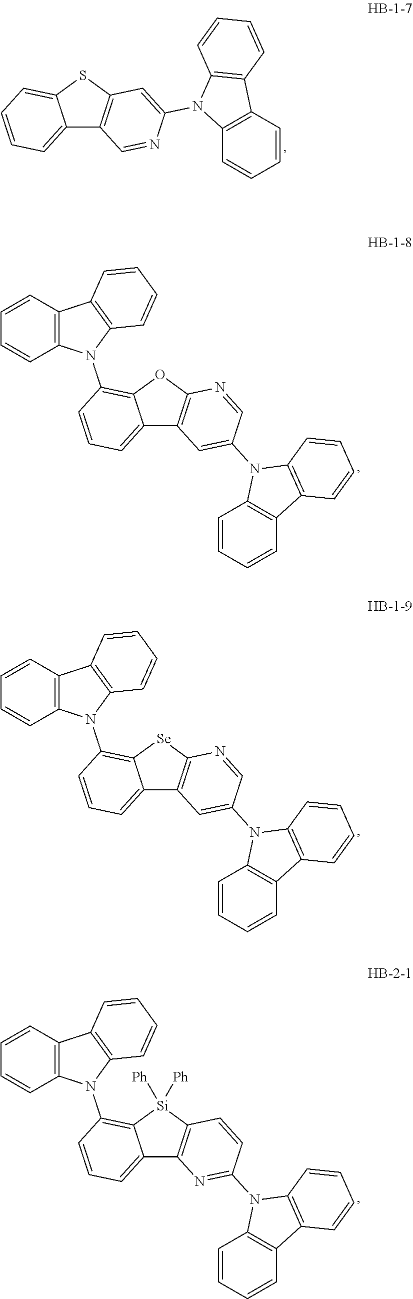 [SMILES: c1ccc2c(c1)sc1cc(-n3c4ccccc4c4ccccc43)ncc12 ; c1cc(-n2c3ccccc3c3ccccc32)c2oc3ncc(-n4c5ccccc5c5ccccc54)cc3c2c1 ; c1cc(-n2c3ccccc3c3ccccc32)c2[se]c3ncc(-n4c5ccccc5c5ccccc54)cc3c2c1 ; c1ccc([Si]2(c3ccccc3)c3ccc(-n4c5ccccc5c5ccccc54)nc3-c3cccc(-n4c5ccccc5c5ccccc54)c32)cc1]

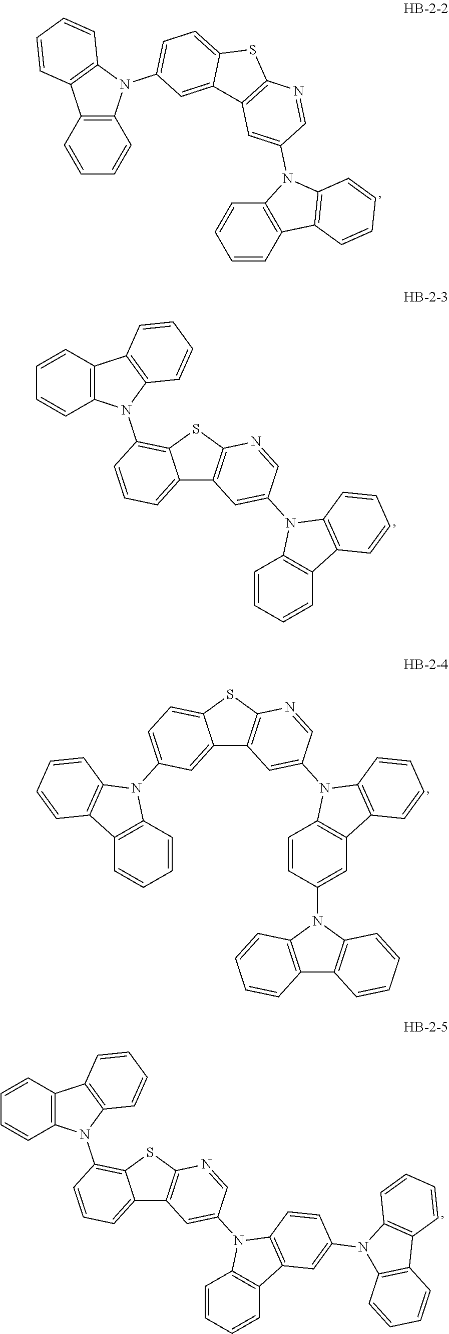 [SMILES: c1ccc2c(c1)c1ccccc1n2-c1ccc2sc3ncc(-n4c5ccccc5c5ccccc54)cc3c2c1 ; c1cc(-n2c3ccccc3c3ccccc32)c2sc3ncc(-n4c5ccccc5c5ccccc54)cc3c2c1 ; c1ccc2c(c1)c1ccccc1n2-c1ccc2sc3ncc(-n4c5ccccc5c5cc(-n6c7ccccc7c7ccccc76)ccc54)cc3c2c1 ; c1cc(-n2c3ccccc3c3ccccc32)c2sc3ncc(-n4c5ccccc5c5cc(-n6c7ccccc7c7ccccc76)ccc54)cc3c2c1]

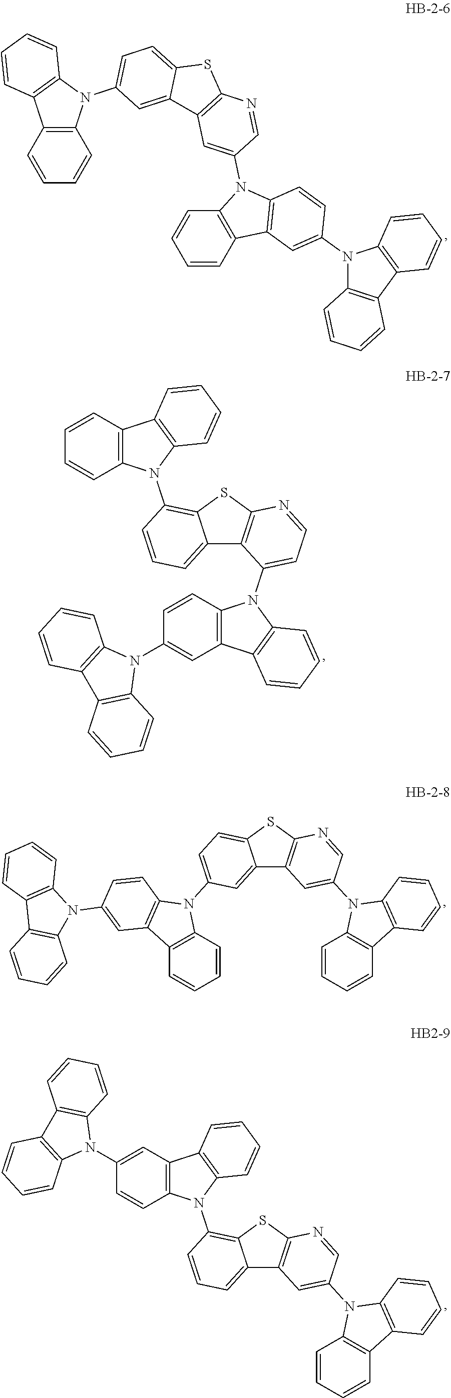 [SMILES: c1ccc2c(c1)c1ccccc1n2-c1ccc2sc3ncc(-n4c5ccccc5c5cc(-n6c7ccccc7c7ccccc76)ccc54)cc3c2c1 ; c1cc(-n2c3ccccc3c3ccccc32)c2sc3nccc(-n4c5ccccc5c5cc(-n6c7ccccc7c7ccccc76)ccc54)c3c2c1 ; c1ccc2c(c1)c1ccccc1n2-c1cnc2sc3ccc(-n4c5ccccc5c5cc(-n6c7ccccc7c7ccccc76)ccc54)cc3c2c1 ; c1cc(-n2c3ccccc3c3cc(-n4c5ccccc5c5ccccc54)ccc32)c2sc3ncc(-n4c5ccccc5c5ccccc54)cc3c2c1]

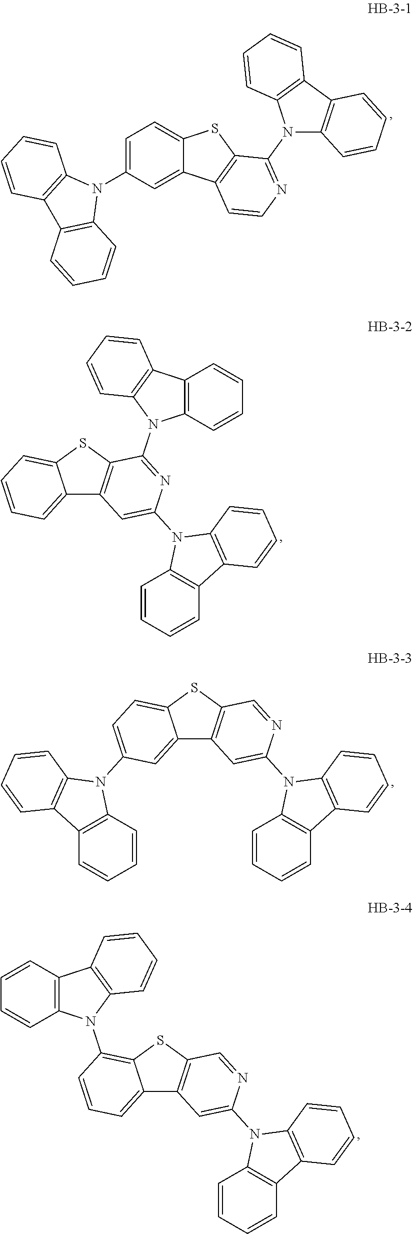 [SMILES: c1ccc2c(c1)c1ccccc1n2-c1ccc2sc3c(-n4c5ccccc5c5ccccc54)nccc3c2c1 ; c1ccc2c(c1)sc1c(-n3c4ccccc4c4ccccc43)nc(-n3c4ccccc4c4ccccc43)cc12 ; c1ccc2c(c1)c1ccccc1n2-c1ccc2sc3cnc(-n4c5ccccc5c5ccccc54)cc3c2c1 ; c1cc(-n2c3ccccc3c3ccccc32)c2sc3cnc(-n4c5ccccc5c5ccccc54)cc3c2c1]

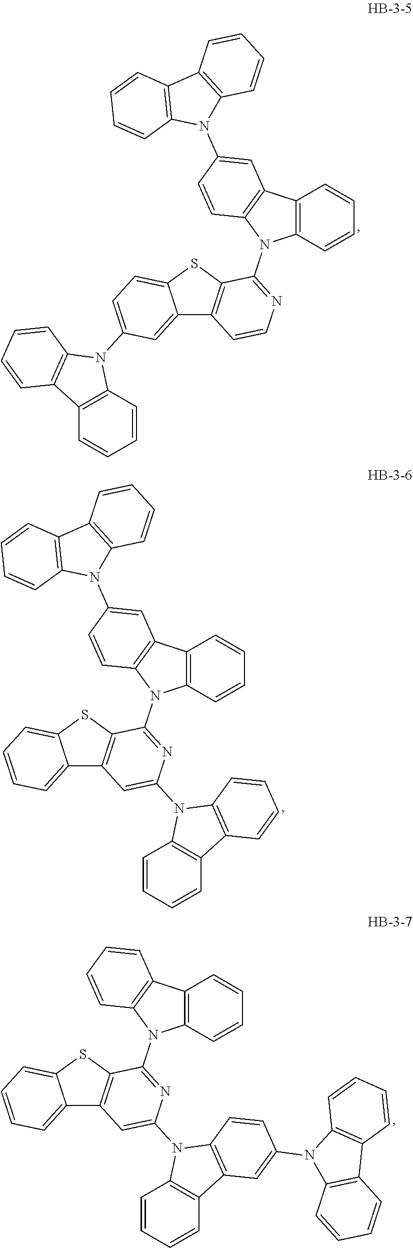

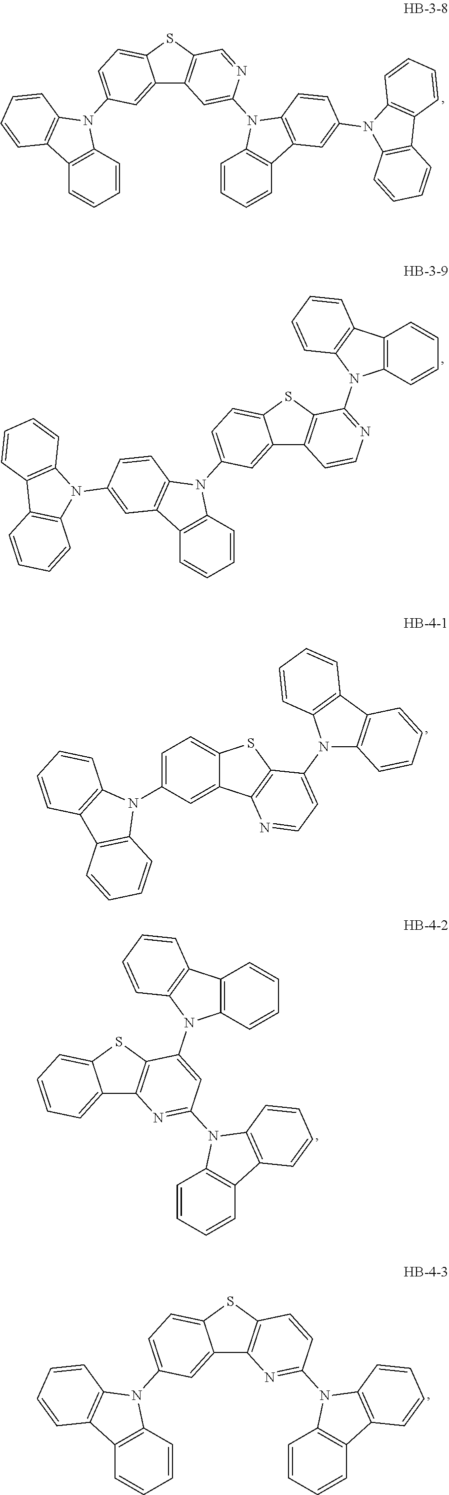

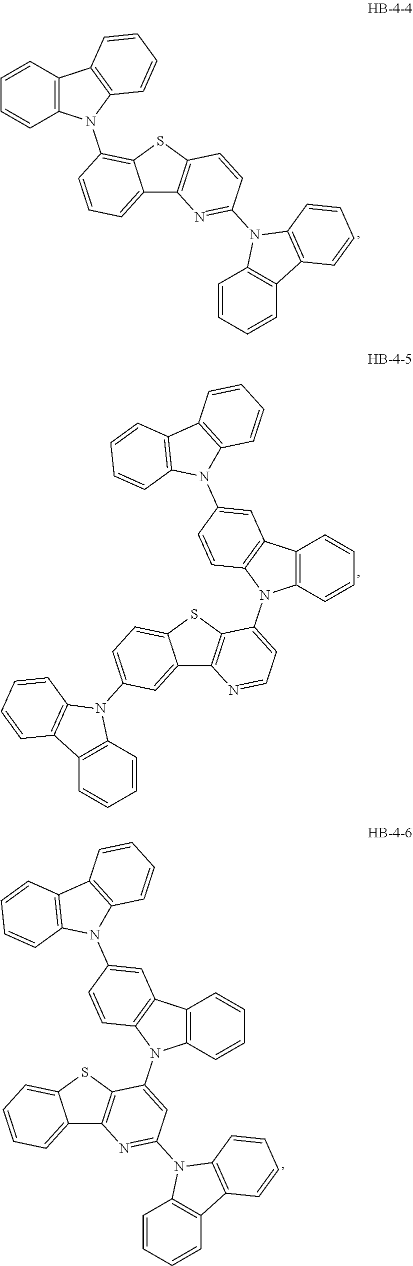

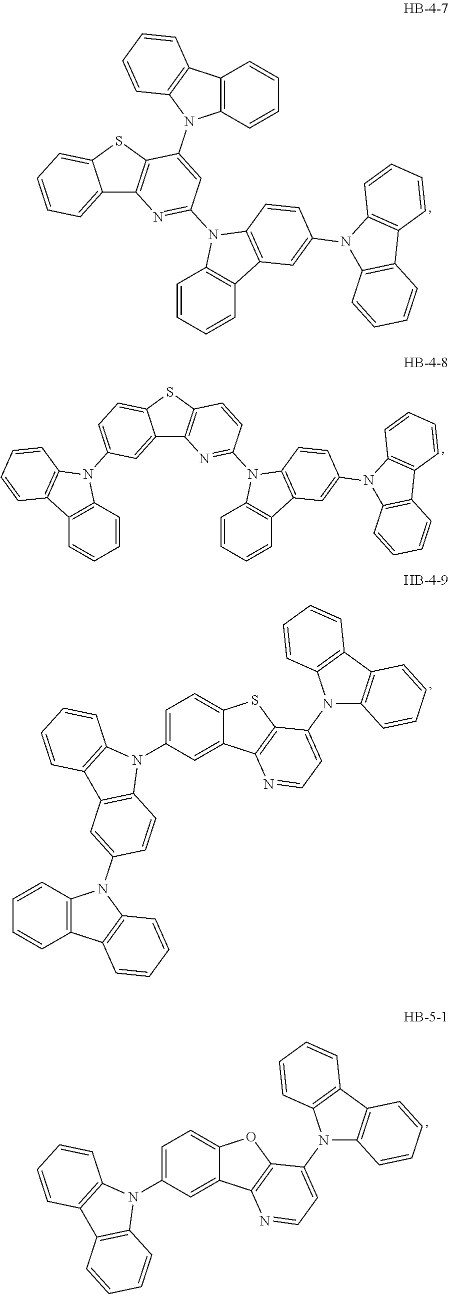 [SMILES: c1ccc2c(c1)sc1c(-n3c4ccccc4c4ccccc43)cc(-n3c4ccccc4c4cc(-n5c6ccccc6c6ccccc65)ccc43)nc12 ; c1ccc2c(c1)c1ccccc1n2-c1ccc2sc3ccc(-n4c5ccccc5c5cc(-n6c7ccccc7c7ccccc76)ccc54)nc3c2c1 ; c1ccc2c(c1)c1ccccc1n2-c1ccc2c(c1)c1ccccc1n2-c1ccc2sc3c(-n4c5ccccc5c5ccccc54)ccnc3c2c1 ; c1ccc2c(c1)c1ccccc1n2-c1ccc2oc3c(-n4c5ccccc5c5ccccc54)ccnc3c2c1]

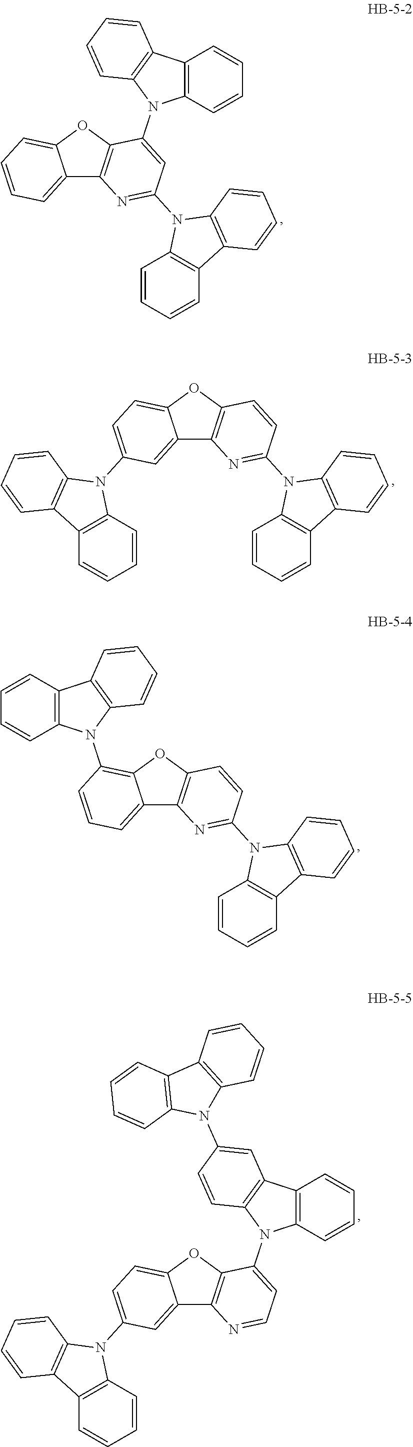 [SMILES: c1ccc2c(c1)oc1c(-n3c4ccccc4c4ccccc43)cc(-n3c4ccccc4c4ccccc43)nc12 ; c1ccc2c(c1)c1ccccc1n2-c1ccc2oc3ccc(-n4c5ccccc5c5ccccc54)nc3c2c1 ; c1cc(-n2c3ccccc3c3ccccc32)c2oc3ccc(-n4c5ccccc5c5ccccc54)nc3c2c1 ; c1ccc2c(c1)c1ccccc1n2-c1ccc2oc3c(-n4c5ccccc5c5cc(-n6c7ccccc7c7ccccc76)ccc54)ccnc3c2c1]

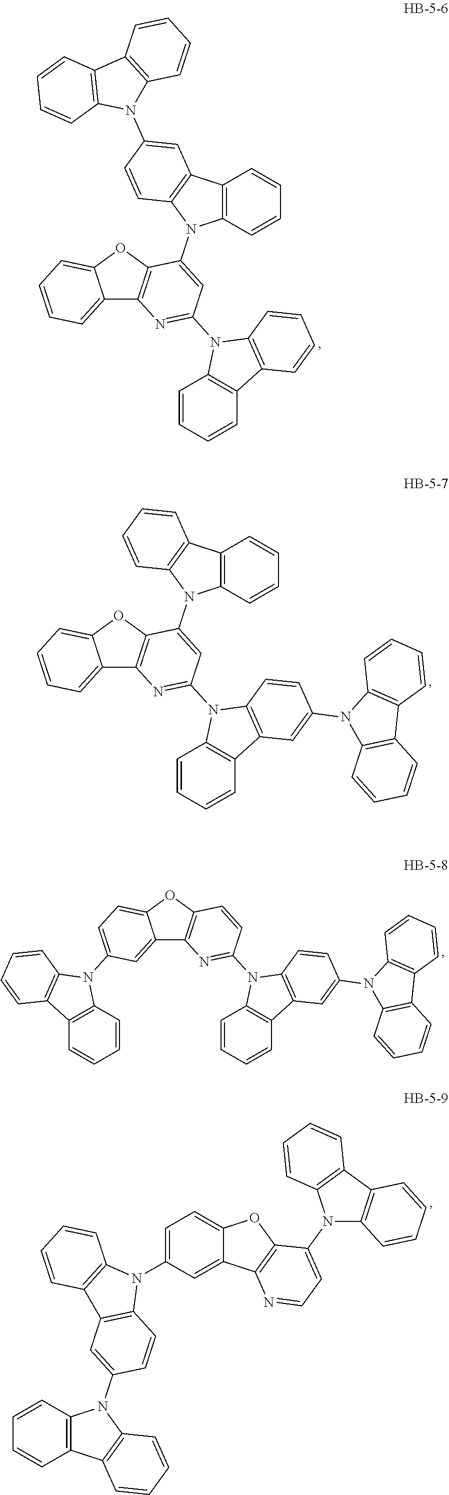 [SMILES: c1ccc2c(c1)oc1c(-n3c4ccccc4c4cc(-n5c6ccccc6c6ccccc65)ccc43)cc(-n3c4ccccc4c4ccccc43)nc12 ; c1ccc2c(c1)oc1c(-n3c4ccccc4c4ccccc43)cc(-n3c4ccccc4c4cc(-n5c6ccccc6c6ccccc65)ccc43)nc12 ; c1ccc2c(c1)c1ccccc1n2-c1ccc2oc3ccc(-n4c5ccccc5c5cc(-n6c7ccccc7c7ccccc76)ccc54)nc3c2c1 ; c1ccc2c(c1)c1ccccc1n2-c1ccc2c(c1)c1ccccc1n2-c1ccc2oc3c(-n4c5ccccc5c5ccccc54)ccnc3c2c1]

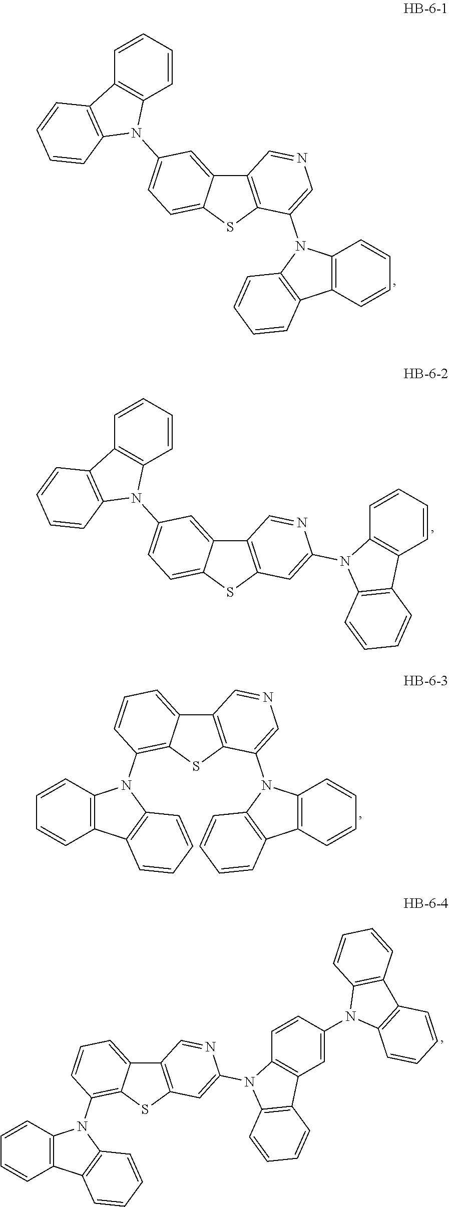 [SMILES: c1ccc2c(c1)c1ccccc1n2-c1ccc2sc3c(-n4c5ccccc5c5ccccc54)cncc3c2c1 ; c1ccc2c(c1)c1ccccc1n2-c1ccc2sc3cc(-n4c5ccccc5c5ccccc54)ncc3c2c1 ; c1cc(-n2c3ccccc3c3ccccc32)c2sc3c(-n4c5ccccc5c5ccccc54)cncc3c2c1 ; c1cc(-n2c3ccccc3c3ccccc32)c2sc3cc(-n4c5ccccc5c5cc(-n6c7ccccc7c7ccccc76)ccc54)ncc3c2c1]

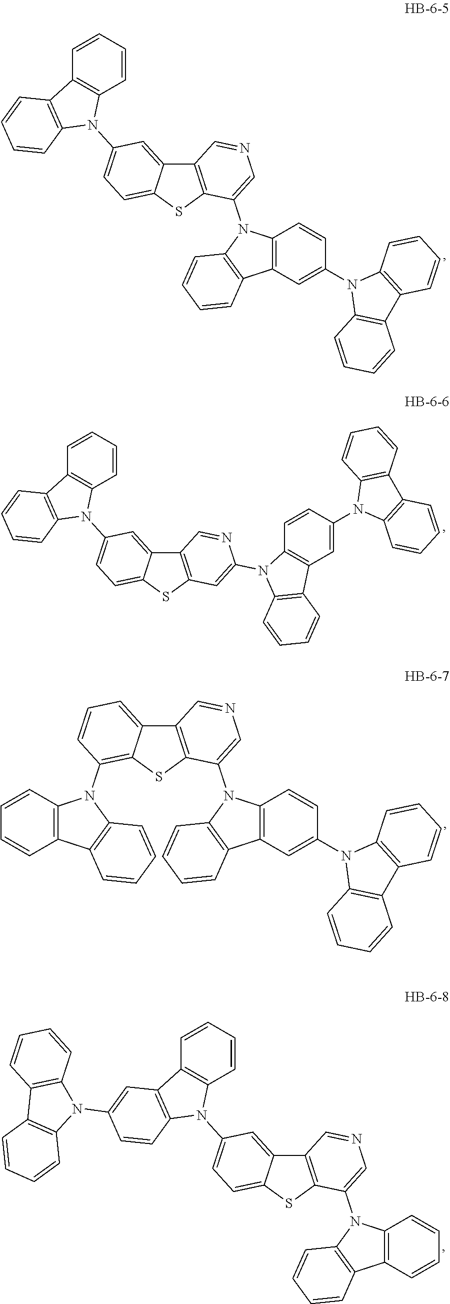 [SMILES: c1ccc2c(c1)c1ccccc1n2-c1ccc2sc3c(-n4c5ccccc5c5cc(-n6c7ccccc7c7ccccc76)ccc54)cncc3c2c1 ; c1ccc2c(c1)c1ccccc1n2-c1ccc2sc3cc(-n4c5ccccc5c5cc(-n6c7ccccc7c7ccccc76)ccc54)ncc3c2c1 ; c1cc(-n2c3ccccc3c3ccccc32)c2sc3c(-n4c5ccccc5c5cc(-n6c7ccccc7c7ccccc76)ccc54)cncc3c2c1 ; c1ccc2c(c1)c1ccccc1n2-c1ccc2c(c1)c1ccccc1n2-c1ccc2sc3c(-n4c5ccccc5c5ccccc54)cncc3c2c1]

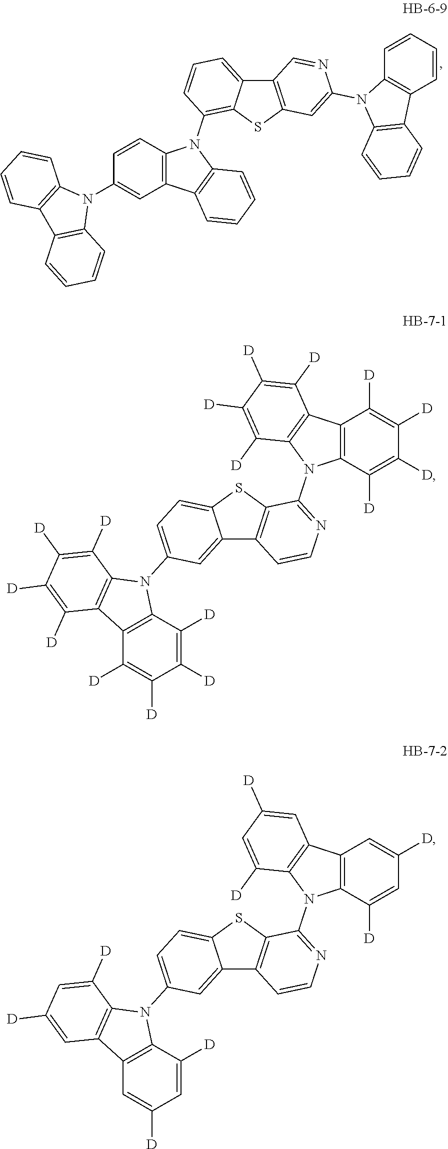

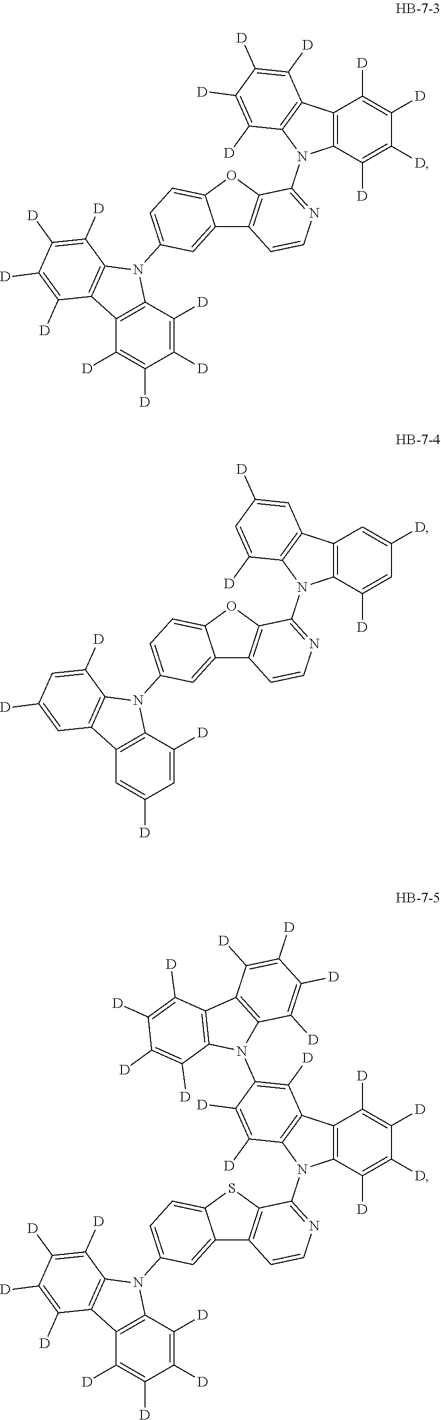

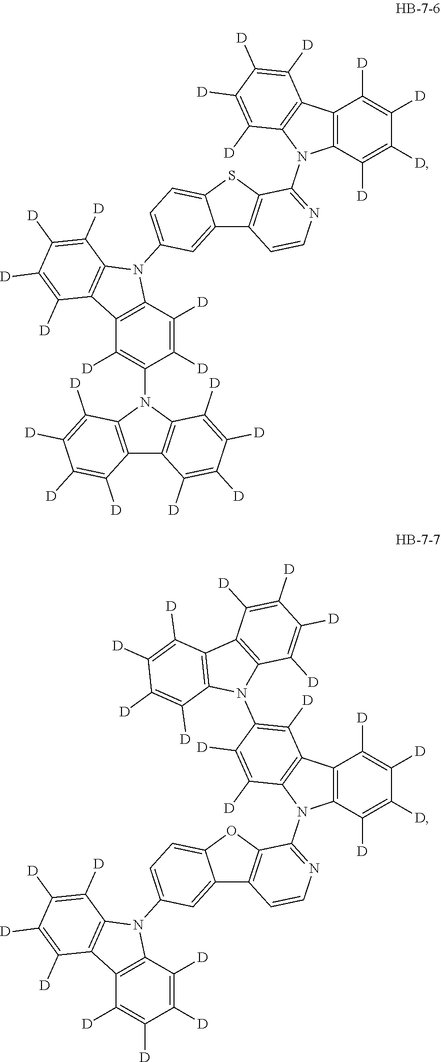

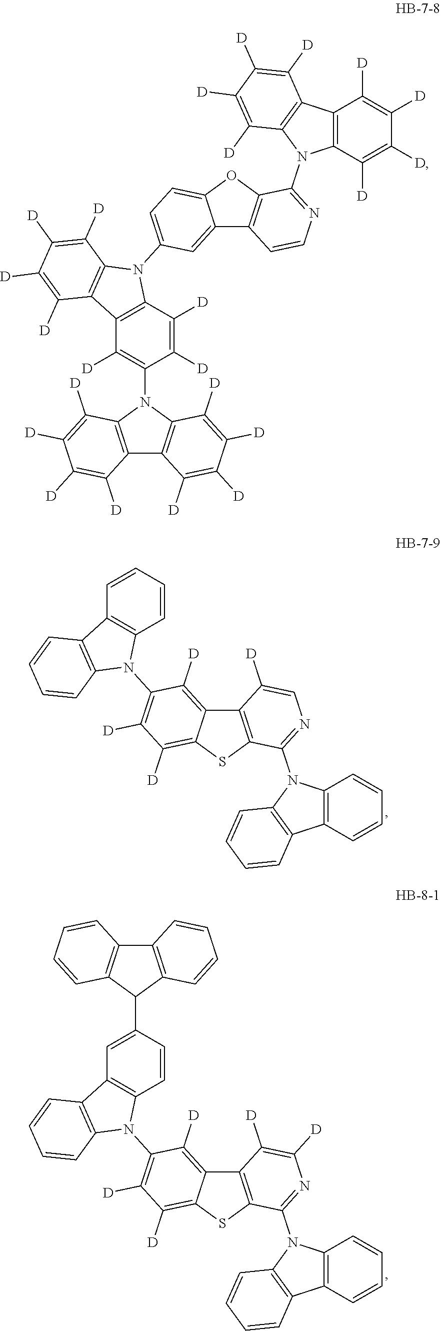

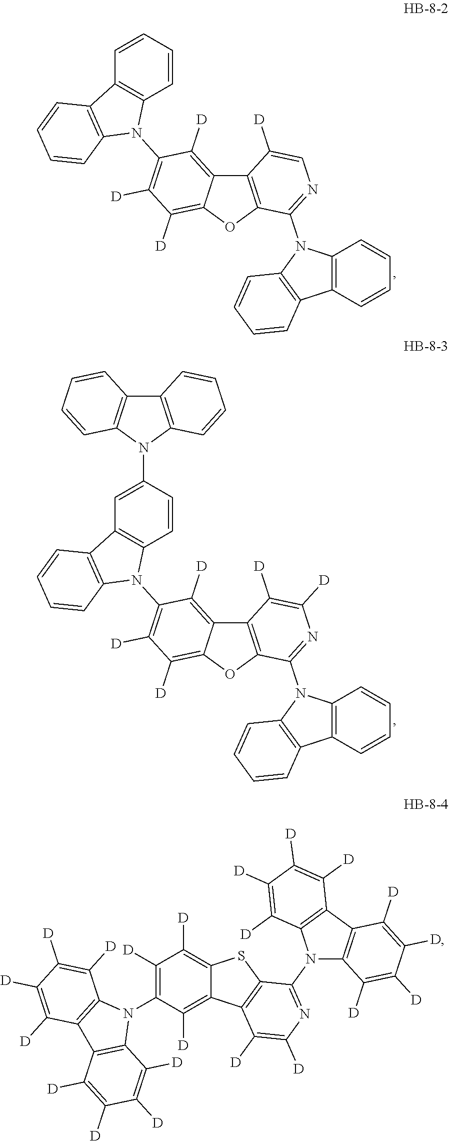

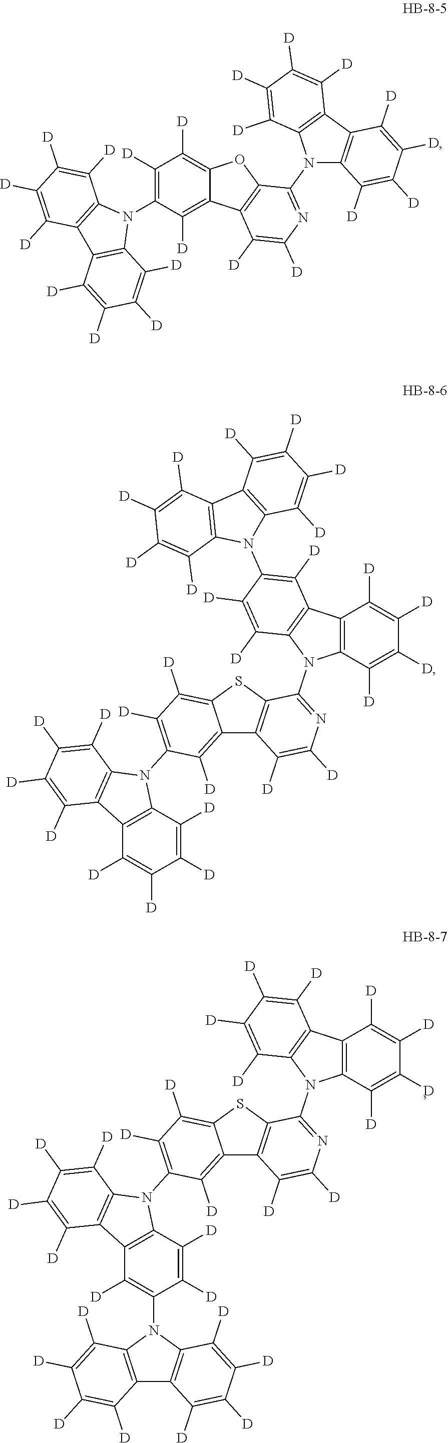

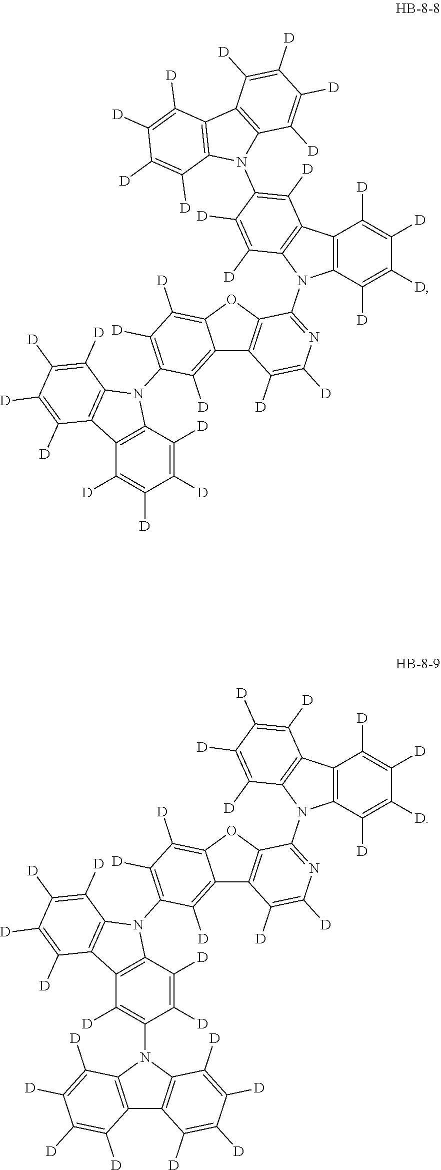

8. The blue delayed fluorescent device according to claim 1, wherein at least one of X₁ and X₂ is selected from N—R_{d1};
preferably, the blue delayed fluorescent material has a structure represented by Formula 5: 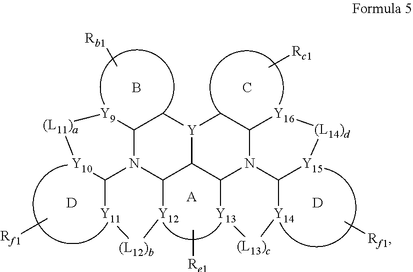
wherein in Formula 5,
the ring A, the ring B, the ring C and the ring D are, at each occurrence identically or differently, selected from an aromatic ring having 6 to 30 carbon atoms, a heteroaromatic ring having 3 to 30 carbon atoms or a combination thereof;
Y is selected from B, P═O, P═S, Al, Ga, As, SiR′ or GeR′;
Y₉ to Y₁₆ are each independently selected from C, CR″ or N;
a, b, c and d are each independently selected from 0 or 1;
L₁₁, L₁₂, L₁₃ and L₁₄ are, at each occurrence identically or differently, selected from a single bond, O, S or NR′″;
R_{b1}, R_{c1}, Rₑ₁ and R_{f1} represent, at each occurrence identically or differently, mono-substitution, multiple substitutions or non-substitution;
R′, R″, R′″, R_{b1}, R_{c1}, Rₑ₁ and R_{f1} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and
adjacent substituents R_{b1}, R_{c1}, Rₑ₁, R_{f1} can be optionally joined to form a ring.

9. The blue delayed fluorescent device according to claim 8, wherein the ring A, the ring B, the ring C and the ring D are, at each occurrence identically or differently, selected from an aromatic ring having 6 to 18 carbon atoms, a heteroaromatic ring having 3 to 18 carbon atoms or a combination thereof;
preferably, the ring A, the ring B, the ring C and the ring D are, at each occurrence identically or differently, selected from a benzene ring, a pyridine ring, a carbazole ring, an N-phenylcarbazole ring, a dibenzofuran ring or a dibenzothiophene ring; and
more preferably, the ring A, the ring B, the ring C and the ring D are, at each occurrence identically or differently, selected from a benzene ring.

10. The blue delayed fluorescent device according to claim 8, wherein Y is selected from B, P═O or P═S; preferably, Y is B.

11. The blue delayed fluorescent device according to claim 8, wherein a+b+c+d is greater than or equal to 1;
preferably, a+b+c+d is greater than or equal to 2;
more preferably, a is 1, b is 0, c is 1, and d is 0; alternatively, a is 1, b is 0, c is 0, and d is 1; and
most preferably, a is 1, b is 0, c is 1, d is 0, and Lu and L₁₃ are selected from a single bond;
alternatively, a is 1, b is 0, c is 0, d is 1, and L₁₁ and L₁₄ are selected from a single bond.

12. The blue delayed fluorescent device according to claim 8, wherein R_{b1}, R_{c1}, Rₑ₁ and R_{f1} are, at each occurrence identically or differently, selected from the group consisting of: hydrogen, deuterium, fluorine, methyl, deuterated methyl, ethyl, n-propyl, isopropyl, cyclopropyl, n-butyl, isobutyl, t-butyl, cyclopentyl, cyclohexyl, phenyl, trimethylsilyl, carbazolyl, indolyl, benzofuryl, dibenzofuryl, benzosilolyl, dibenzosilolyl, benzothienyl, dibenzothienyl, dibenzoselenophene and combinations thereof.

13. The blue delayed fluorescent device according to claim 1, wherein the blue delayed fluorescent material is selected from the group consisting of the following:

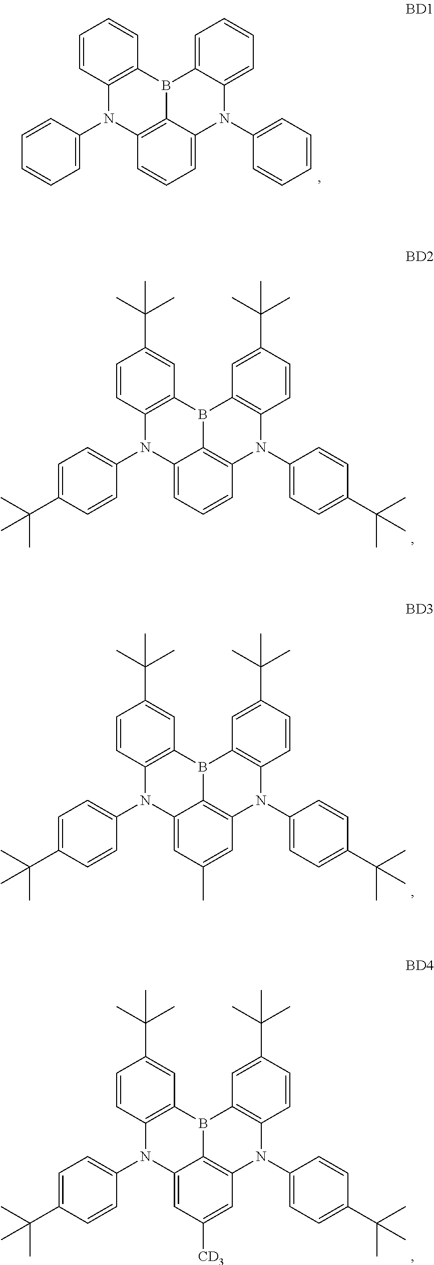

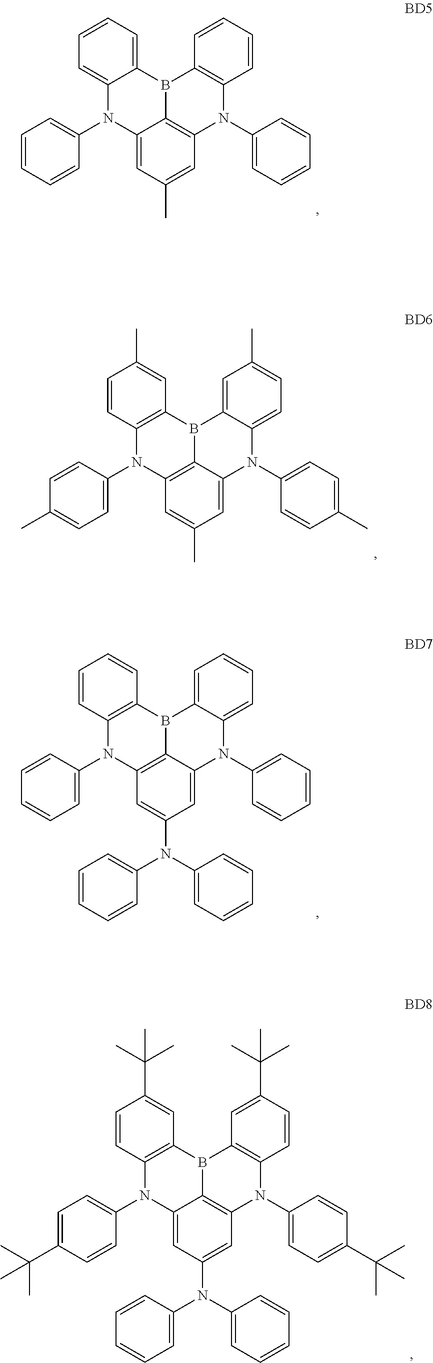

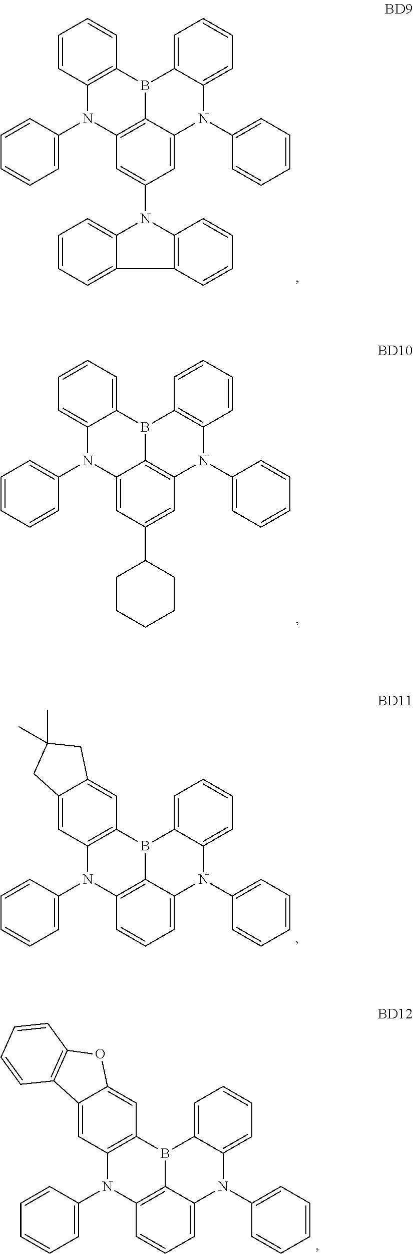 [SMILES: c1ccc(N2c3ccccc3B3c4ccccc4N(c4ccccc4)c4cc(-n5c6ccccc6c6ccccc65)cc2c43)cc1 ; c1ccc(N2c3ccccc3B3c4ccccc4N(c4ccccc4)c4cc(C5CCCCC5)cc2c43)cc1 ; CC1(C)Cc2cc3c(cc2C1)N(c1ccccc1)c1cccc2c1B3c1ccccc1N2c1ccccc1 ; c1ccc(N2c3ccccc3B3c4cc5oc6ccccc6c5cc4N(c4ccccc4)c4cccc2c43)cc1]

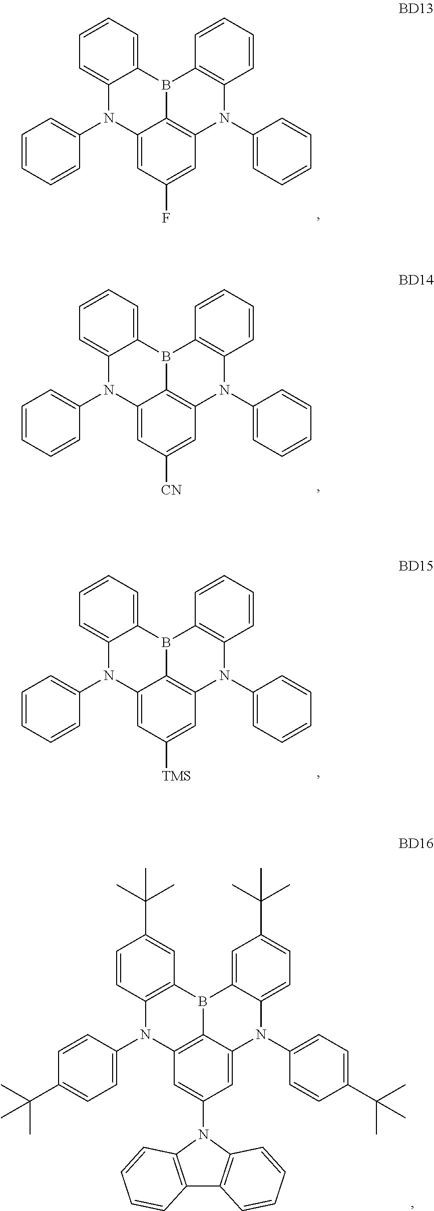 [SMILES: Fc1cc2c3c(c1)N(c1ccccc1)c1ccccc1B3c1ccccc1N2c1ccccc1 ; N#Cc1cc2c3c(c1)N(c1ccccc1)c1ccccc1B3c1ccccc1N2c1ccccc1 ; C[Si](C)(C)c1cc2c3c(c1)N(c1ccccc1)c1ccccc1B3c1ccccc1N2c1ccccc1 ; CC(C)(C)c1ccc(N2c3ccc(C(C)(C)C)cc3B3c4cc(C(C)(C)C)ccc4N(c4ccc(C(C)(C)C)cc4)c4cc(-n5c6ccccc6c6ccccc65)cc2c43)cc1]

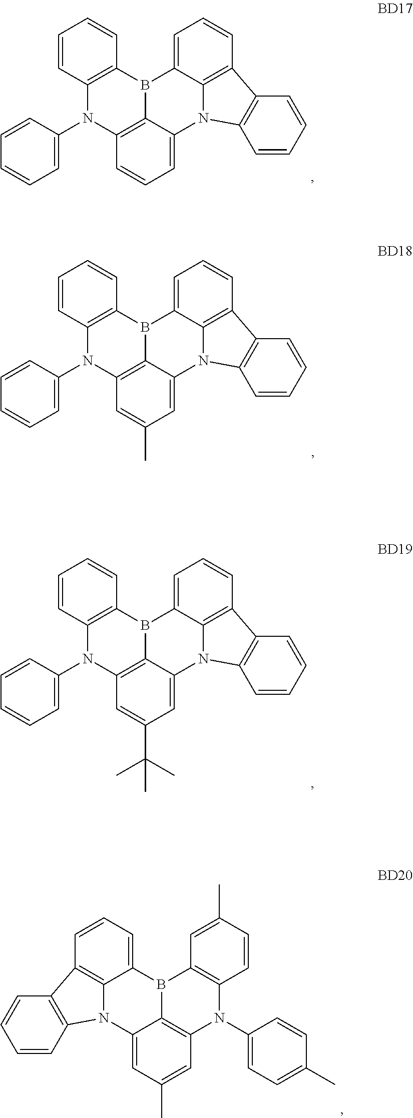 [SMILES: c1ccc(N2c3ccccc3B3c4c2cccc4-n2c4ccccc4c4cccc3c42)cc1 ; Cc1cc2c3c(c1)-n1c4ccccc4c4cccc(c41)B3c1ccccc1N2c1ccccc1 ; CC(C)(C)c1cc2c3c(c1)-n1c4ccccc4c4cccc(c41)B3c1ccccc1N2c1ccccc1 ; Cc1ccc(N2c3ccc(C)cc3B3c4c2cc(C)cc4-n2c4ccccc4c4cccc3c42)cc1]

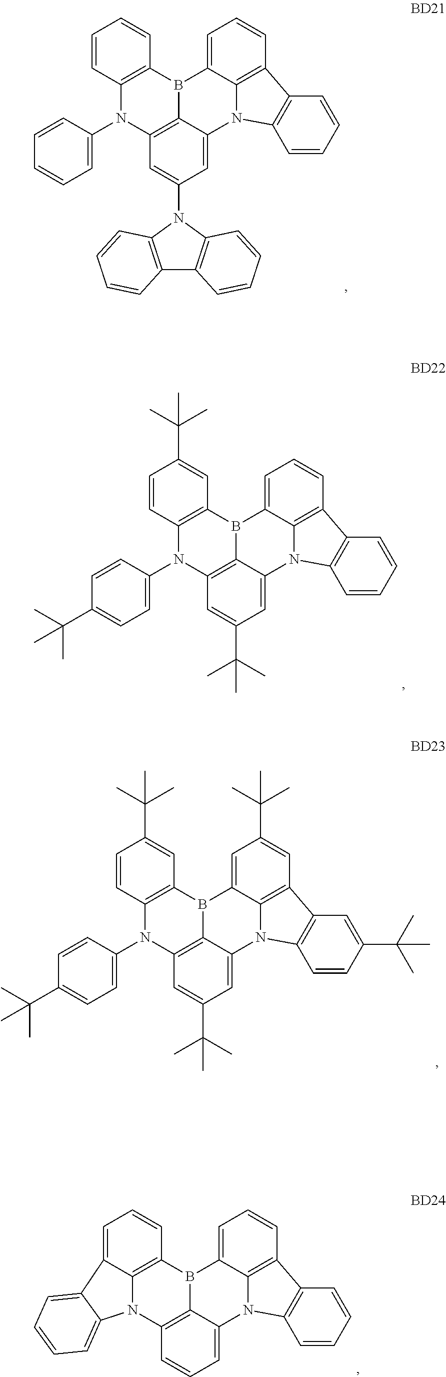 [SMILES: c1ccc(N2c3ccccc3B3c4c2cc(-n2c5ccccc5c5ccccc52)cc4-n2c4ccccc4c4cccc3c42)cc1 ; CC(C)(C)c1ccc(N2c3ccc(C(C)(C)C)cc3B3c4c2cc(C(C)(C)C)cc4-n2c4ccccc4c4cccc3c42)cc1 ; CC(C)(C)c1ccc(N2c3ccc(C(C)(C)C)cc3B3c4c2cc(C(C)(C)C)cc4-n2c4ccc(C(C)(C)C)cc4c4cc(C(C)(C)C)cc3c42)cc1 ; c1cc2c3c(c1)-n1c4ccccc4c4cccc(c41)B3c1cccc3c4ccccc4n-2c13]

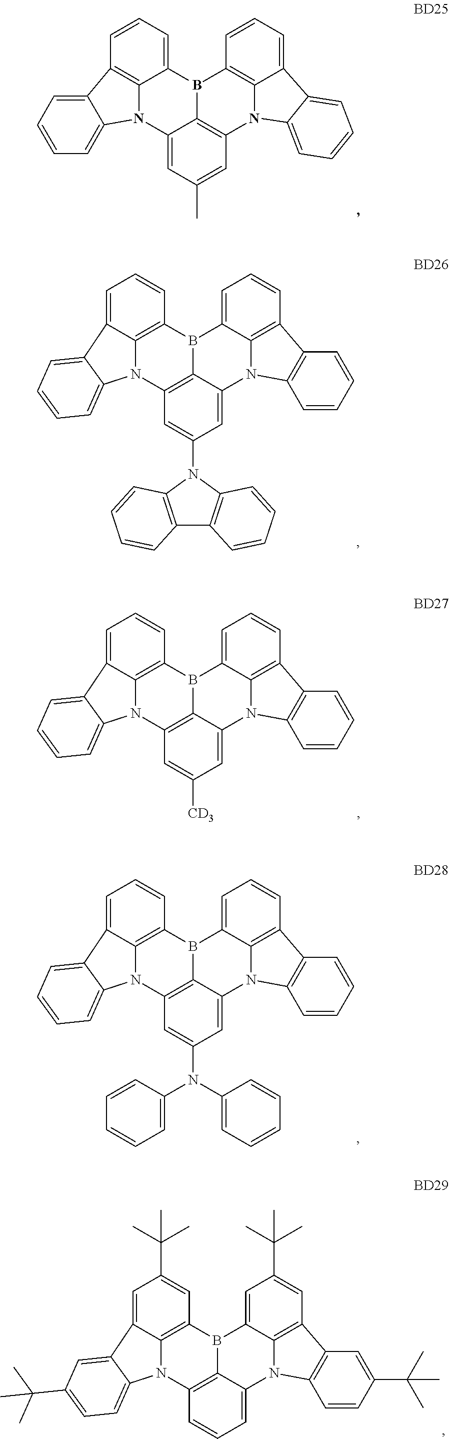 [SMILES: Cc1cc2c3c(c1)-n1c4ccccc4c4cccc(c41)B3c1cccc3c4ccccc4n-2c13 ; c1ccc2c(c1)c1ccccc1n2-c1cc2c3c(c1)-n1c4ccccc4c4cccc(c41)B3c1cccc3c4ccccc4n-2c13 ; [2H]C([2H])([2H])c1cc2c3c(c1)-n1c4ccccc4c4cccc(c41)B3c1cccc3c4ccccc4n-2c13 ; c1ccc(N(c2ccccc2)c2cc3c4c(c2)-n2c5ccccc5c5cccc(c52)B4c2cccc4c5ccccc5n-3c24)cc1 ; CC(C)(C)c1ccc2c(c1)c1cc(C(C)(C)C)cc3c1n2-c1cccc2c1B3c1cc(C(C)(C)C)cc3c4cc(C(C)(C)C)ccc4n-2c13]

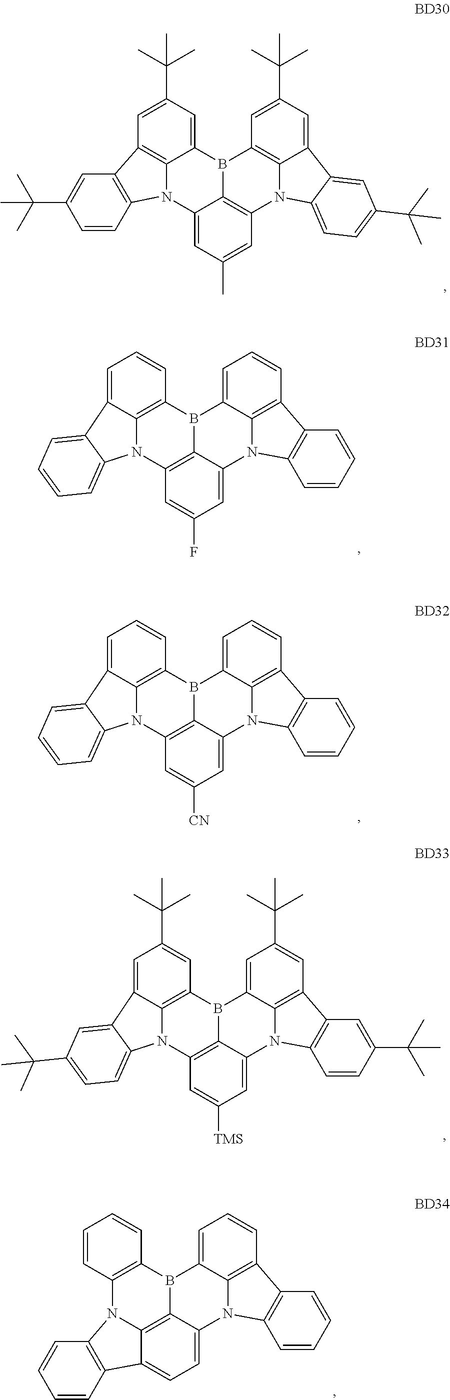

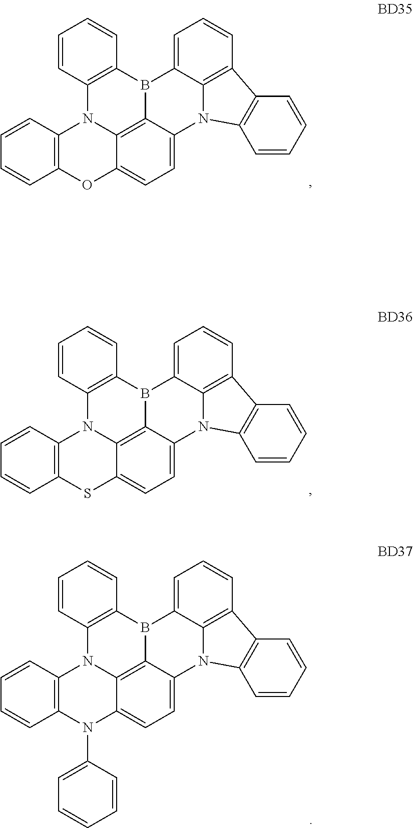 [SMILES: c1ccc2c(c1)Oc1ccc3c4c1N2c1ccccc1B4c1cccc2c4ccccc4n-3c12 ; c1ccc2c(c1)Sc1ccc3c4c1N2c1ccccc1B4c1cccc2c4ccccc4n-3c12 ; c1ccc(N2c3ccccc3N3c4ccccc4B4c5c(ccc2c53)-n2c3ccccc3c3cccc4c32)cc1]

14. The blue delayed fluorescent device according to claim 1, wherein the triplet energy level of the first host material and the triplet energy level of the second host material are each greater than 2.69 eV.

15. The blue delayed fluorescent device according to claim 1, wherein the first host material has a structure represented by any one of Formula 6 to Formula 8: 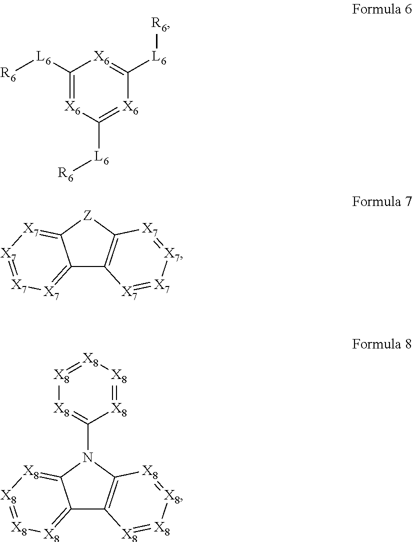
wherein,
X₆, X₇ and X₈ shown in Formula 6 to Formula 8 are, at each occurrence identically or differently, selected from CR₇ or N; at least one of X₆ shown in Formula 6 is selected from N, at least one of X₇ shown in Formula 7 is selected from N, and at least one of X₈ shown in Formula 8 is selected from N;
Z is, at each occurrence identically or differently, selected from O or S;
L₆ is, at each occurrence identically or differently, selected from the group consisting of: a single bond, substituted or unsubstituted arylene having 6 to 30 carbon atoms, substituted or unsubstituted heteroarylene having 3 to 30 carbon atoms and combinations thereof;
R₆ and R₇ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and
adjacent substituents R₇ can be optionally joined to form a ring.

16. The blue delayed fluorescent device according to claim 1, wherein the first host material is selected from the group consisting of the following:

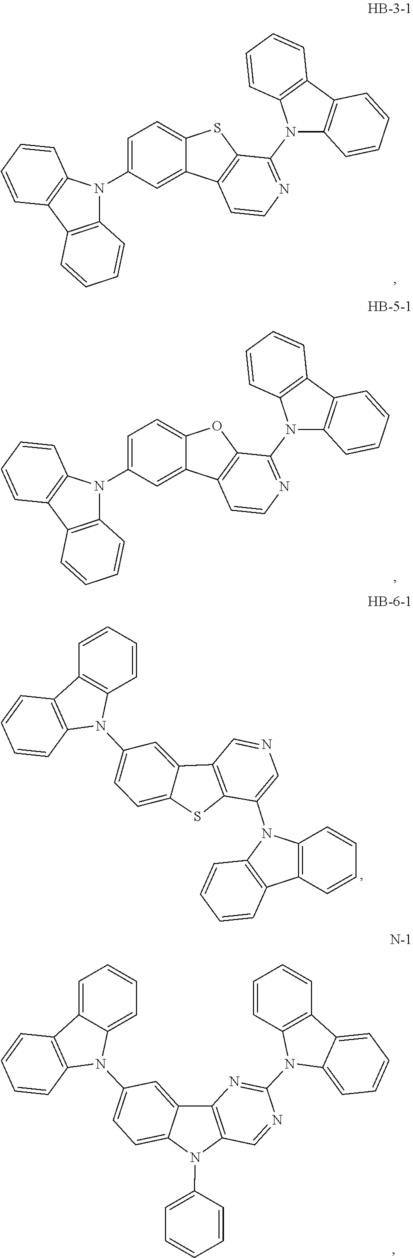 [SMILES: c1ccc2c(c1)c1ccccc1n2-c1ccc2sc3c(-n4c5ccccc5c5ccccc54)nccc3c2c1 ; c1ccc2c(c1)c1ccccc1n2-c1ccc2oc3c(-n4c5ccccc5c5ccccc54)nccc3c2c1 ; c1ccc2c(c1)c1ccccc1n2-c1ccc2sc3c(-n4c5ccccc5c5ccccc54)cncc3c2c1 ; c1ccc(-n2c3ccc(-n4c5ccccc5c5ccccc54)cc3c3nc(-n4c5ccccc5c5ccccc54)ncc32)cc1]

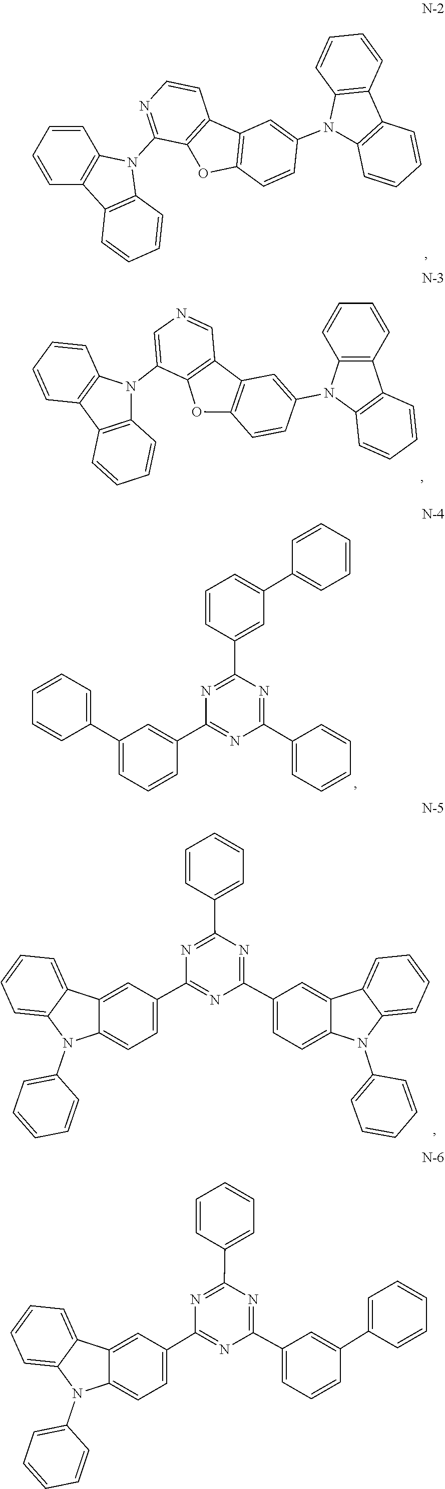 [SMILES: c1ccc2c(c1)c1ccccc1n2-c1ccc2oc3c(-n4c5ccccc5c5ccccc54)nccc3c2c1 ; c1ccc2c(c1)c1ccccc1n2-c1ccc2oc3c(-n4c5ccccc5c5ccccc54)cncc3c2c1 ; c1ccc(-c2cccc(-c3nc(-c4ccccc4)nc(-c4cccc(-c5ccccc5)c4)n3)c2)cc1 ; c1ccc(-c2nc(-c3ccc4c(c3)c3ccccc3n4-c3ccccc3)nc(-c3ccc4c(c3)c3ccccc3n4-c3ccccc3)n2)cc1 ; c1ccc(-c2cccc(-c3nc(-c4ccccc4)nc(-c4ccc5c(c4)c4ccccc4n5-c4ccccc4)n3)c2)cc1]

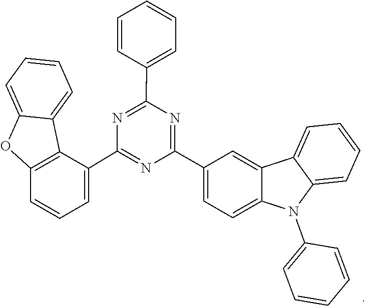 [SMILES: c1ccc(-c2nc(-c3ccc4c(c3)c3ccccc3n4-c3ccccc3)nc(-c3cccc4oc5ccccc5c34)n2)cc1]

17. The blue delayed fluorescent device according to claim 1, wherein the second host material has a structure represented by Formula 9: 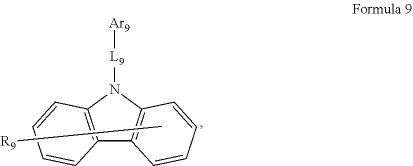
wherein in Formula 9,
L₉ is selected from a single bond, substituted or unsubstituted arylene having 6 to 30 carbon atoms, substituted or unsubstituted heteroarylene having 3 to 30 carbon atoms or a combination thereof;
Ar₉ is selected from substituted or unsubstituted aryl having 6 to 30 carbon atoms, substituted or unsubstituted heteroaryl having 3 to 30 carbon atoms, substituted or unsubstituted amino having 0 to 30 carbon atoms or a combination thereof;
R₉ represents, at each occurrence identically or differently, mono-substitution, multiple substitutions or non-substitution;
R₉ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and
adjacent substituents R₉ can be optionally joined to form a ring.

18. The blue delayed fluorescent device according to claim 1, wherein the second host material is selected from the group consisting of the following:

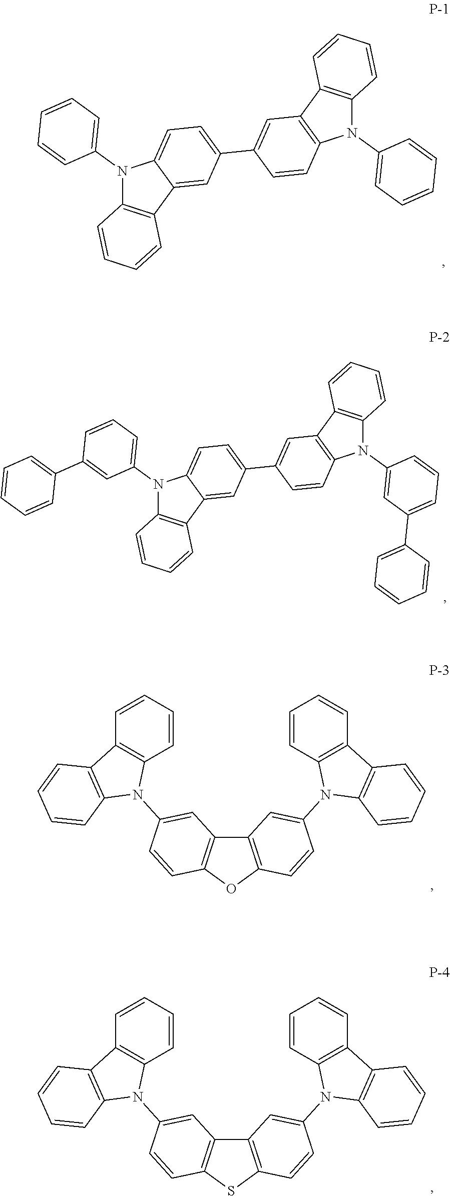 [SMILES: c1ccc(-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5cccc(-c6ccccc6)c5)ccc43)c2)cc1 ; c1ccc2c(c1)c1ccccc1n2-c1ccc2oc3ccc(-n4c5ccccc5c5ccccc54)cc3c2c1 ; c1ccc2c(c1)c1ccccc1n2-c1ccc2sc3ccc(-n4c5ccccc5c5ccccc54)cc3c2c1]

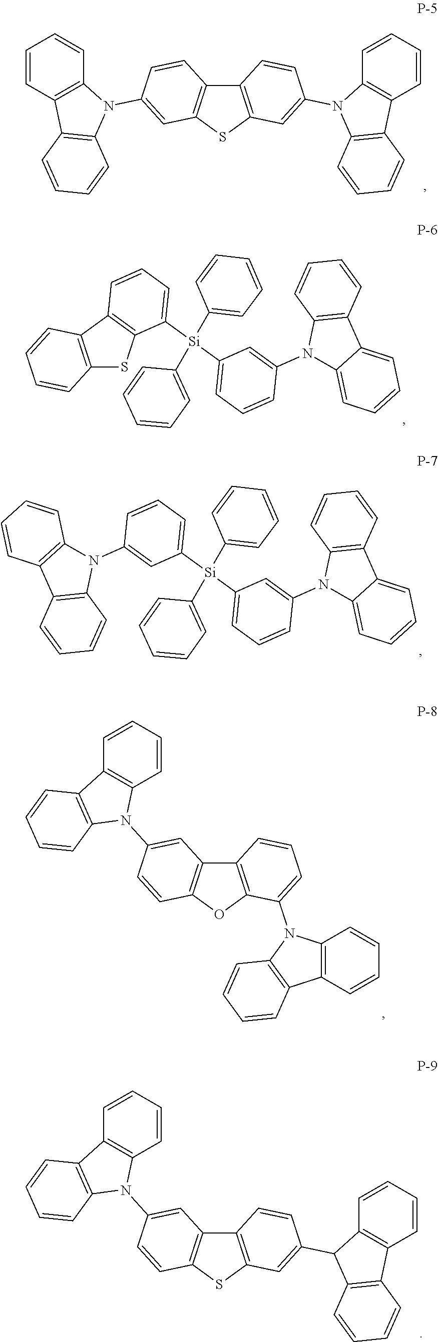 [SMILES: c1ccc2c(c1)c1ccccc1n2-c1ccc2c(c1)sc1cc(-n3c4ccccc4c4ccccc43)ccc12 ; c1ccc([Si](c2ccccc2)(c2cccc(-n3c4ccccc4c4ccccc43)c2)c2cccc3c2sc2ccccc23)cc1 ; c1ccc([Si](c2ccccc2)(c2cccc(-n3c4ccccc4c4ccccc43)c2)c2cccc(-n3c4ccccc4c4ccccc43)c2)cc1 ; c1cc(-n2c3ccccc3c3ccccc32)c2oc3ccc(-n4c5ccccc5c5ccccc54)cc3c2c1 ; c1ccc2c(c1)-c1ccccc1C2c1ccc2c(c1)sc1ccc(-n3c4ccccc4c4ccccc43)cc12]

19. A light-emitting device, comprising an anode, a cathode and a light-emitting unit disposed between the anode and the cathode;
wherein the light-emitting unit at least comprises a blue delayed fluorescent unit, a green phosphorescent unit and a red phosphorescent unit;
wherein the blue delayed fluorescent unit, the green phosphorescent unit and the red phosphorescent unit each comprise an emissive layer and a hole blocking layer disposed between the emissive layer and the cathode;
wherein the hole blocking layers comprise a same hole blocking material or different hole blocking materials;
wherein the hole blocking material is a compound having a structure represented by Formula 1: 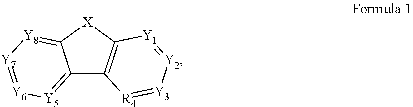
wherein in Formula 1,
X is selected from O, S, Se, SiRR or PR;
R is, at each occurrence identically or differently, selected from the group consisting of: substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted aryl having 6 to 30 carbon atoms, and substituted or unsubstituted heteroaryl having 3 to 30 carbon atoms; when multiple R are present at the same time, the multiple R are the same or different;
Y₁ to Y₈ are, at each occurrence identically or differently, selected from CR₁, CR₂ or N; at least one of Y₁ to Y₈ is selected from N, and at least one of Y₁ to Y₈ is selected from CR₁;
R₁ has a structure represented by Formula 2: 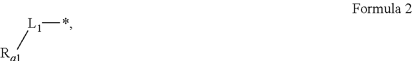
wherein Rₐ₁ is, at each occurrence identically or differently, selected from substituted or unsubstituted aryl having 6 to 30 carbon atoms, substituted or unsubstituted heteroaryl having 3 to 30 carbon atoms or a combination thereof;
L₁ is, at each occurrence identically or differently, selected from a single bond, substituted or unsubstituted arylene having 6 to 30 carbon atoms, substituted or unsubstituted heteroarylene having 3 to 30 carbon atoms or a combination thereof; [symbol]
represents a position where the substituent having the structure of Formula 2 is joined to Formula 1;
R₂ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
adjacent substituents R, R₂ can be optionally joined to form a ring;
the emissive layer of the blue delayed fluorescent unit comprises a first host material, a second host material and a blue delayed fluorescent material, wherein the triplet energy level of the first host material and the triplet energy level of the second host material are each greater than that of the blue delayed fluorescent material;
the blue delayed fluorescent material has a structure represented by Formula 3: 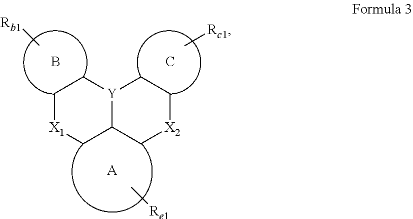
wherein in Formula 3,
the ring A, the ring B and the ring C are each independently selected from an aromatic ring having 6 to 30 carbon atoms, a heteroaromatic ring having 3 to 30 carbon atoms or a combination thereof;
Y is selected from B, P═O, P═S, Al, Ga, As, SiR′ or GeR′;
X₁ and X₂ are each independently selected from O, N—R_{d1}, S or Se, wherein N—R_{d1} has a structure represented by Formula 4: 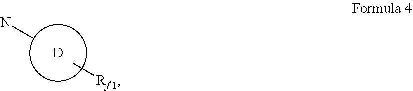
wherein the ring D is, at each occurrence identically or differently, selected from an aromatic ring having 6 to 30 carbon atoms, a heteroaromatic ring having 3 to 30 carbon atoms or a combination thereof;
R_{b1}, R_{c1}, Rₑ₁ and R_{f1} represent, at each occurrence identically or differently, mono-substitution, multiple substitutions or non-substitution;
R′, R_{b1}, R_{c1}, Rₑ₁ and R_{f1} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
adjacent substituents R_{b1}, R_{c1}, Rₑ₁, R_{f1} can be optionally joined to form a ring;
the emissive layer of the green phosphorescent unit comprises a green phosphorescent material; and
the emissive layer of the red phosphorescent unit comprises a red phosphorescent material.

20. The light-emitting device according to claim 19, wherein the blue delayed fluorescent unit, the green phosphorescent unit and the red phosphorescent unit share a hole blocking layer, wherein the hole blocking layer comprises a same hole blocking material.

21. A display assembly, comprising the device according to claim 1.
